(19) United States
(12) Patent Application Publication
(10) Pub. No.: US 2026/0240757 A1
(43) Pub. Date: Aug. 20, 2026

(54) ORAL DRUG DOSAGE FORMS FOR COLON DELIVERY AND METHODS OF USE AND MAKING THEREOF

(71) Applicant: Triastek, Inc., Nanjing (CN)

(72) Inventors: Xianghao ZUO, Nanjing (CN); Huan CHI, Nanjing (CN); Feihuang DENG, Nanjing (CN); Xin LIU, Nanjing (CN); Senping CHENG, Nanjing (CN); Xiaoling LI, Dublin, CA (US)

(21) Appl. No.: 19/165,419

(22) PCT Filed: Mar. 29, 2024
(86) PCT No.: PCT/CN2024/084829

§ 371 Date: Sep. 15, 2025

(30) Foreign Application Priority Data
Mar. 31, 2023 (WO) PCT/CN2023/085458
Mar. 25, 2024 (CN) 202410351921.6

(51) Int. Cl. A61K 9/00; A61K 9/28; A61K 45/06

(52) U.S. Cl. CPC A61K 9/0002; A61K 9/0053; A61K 9/2846; A61K 9/2866; A61K 45/06

(57) ABSTRACT

Provided, in some aspects, is directed to oral drug dosage forms configured to release a drug at a desired location in the colon of an individual. As provided in certain aspects, the oral drug dosage forms comprise a drug component comprising an erodible material admixed with the drug, and a delay component not admixed with the drug, wherein the delay component is configured to prevent release of the drug from the drug component until the oral drug dosage form reaches the colon of the individual following administration. In other aspects, the provided is directed to methods of designing, methods of making, such as using three-dimensional printing, and methods of treatment associated with the oral drug dosage forms described herein.

### TECHNICAL FIELD

The present disclosure, in some aspects, is directed to oral drug dosage forms configured to release a drug at a desired location in the colon of an individual. In other aspects, the present disclosure is directed to methods of designing, methods of making, such as using three-dimensional printing, and methods of treatment associated with the oral drug dosage forms described herein.

### BACKGROUND

Oral administration of pharmaceutical agents provides many patient conveniences, including limiting visits to the doctor and avoiding more invasive drug delivery approaches such as injection or rectal administration. The colon can be a site of treatment and/or a site of drug update, however, the location of the colon near the end of the gastrointestinal tract complicates effective oral administration for delivery to the colon. For example, an ingested oral drug dosage form must endure harsh pH conditions in the stomach, as well as vastly different pH conditions downstream of the stomach, and mechanical stress applied via digestive processes prior to reaching the colon. Capabilities of conventional colon-targeting oral drug dosage forms vary in specificity and obtainable release profiles. For example, some conventional colon-targeting oral drug dosage forms continuously release the drug throughout the gastrointestinal tract before the oral drug dosage form arrives in the colon. This results in reduced drug availability in the colon, limited applicability to active agents that can be delivered, and increased likelihood of systemic adverse effects due to off-target drug release. Thus, there remains a need in the field for oral drug dosage forms capable of precise colon delivery.

### BRIEF SUMMARY

In certain aspects, provided herein is an oral drug dosage form configured to release a drug at a desired location in the colon of an individual, the oral drug dosage form comprising: a drug component comprising an erodible material admixed with the drug; and a delay component not admixed with the drug, wherein the delay component is configured to prevent release of the drug from the drug component until the oral drug dosage form reaches the colon of the individual following administration, wherein the delay component comprises: a pH-based enteric member comprising an erodible material configured to erode at or above a pre-determined pH value.

In some embodiments, the delay component further comprises an erodible delay member, and wherein the oral drug dosage form is configured such that the pH-based enteric member erodes prior to the erodible delay member. In some embodiments, the pH-based enteric member prevents erosion, at least in part, of the erodible delay member, and the erodible delay member prevents erosion, at least in part, of the drug component.

In some embodiments, the oral drug dosage form further comprises a second drug. In some embodiments, the second drug is admixed with the drug component. In some embodiments, the oral drug dosage form further comprises a second drug component comprising an erodible material admixed with the second drug, wherein the delay component is configured to prevent release of the second drug from the second drug component until the oral drug dosage form reaches the colon of the individual following administration. In some embodiments, the delay component comprises a second erodible delay member, wherein the oral drug dosage form is configured such that the pH-based enteric member erodes prior to the erodible delay member, the erodible delay member erodes prior to the second drug component, the second drug component erodes prior to the second erodible delay member, and the second erodible delay member erodes prior to the drug component. In some embodiments, the pH-based enteric member prevents erosion, at least in part, of the erodible delay member, the erodible delay member prevents erosion, at least in part, of the second drug component, the second drug component prevents erosion, at least in part, of the second erodible delay member, and the second erodible delay member prevents erosion, at least in part, of the drug component. In some embodiments, the erodible material of the erodible delay member and the erodible material of the second erodible delay member are the same. In some embodiments, the erodible material of the erodible delay member and the erodible material of the second erodible delay member are different.

In some embodiments, the oral drug dosage form further comprises a bacterial degradation moiety. In some embodiments, the bacterial degradation moiety is admixed with the erodible material of the erodible delay member. In some embodiments, the bacterial degradation moiety is admixed with the drug component. In some embodiments, the delay component further comprises a bacterial degradation member comprising an erodible material admixed with the bacterial degradation moiety, wherein the oral drug dosage form is configured such that the pH-based enteric member erodes prior to the erodible delay member, the erodible delay member erodes prior to the bacterial degradation member, and the bacterial degradation member erodes prior to the drug component. In some embodiments, the pH-based enteric member prevents erosion, at least in part, of the erodible delay member, the erodible delay member prevents erosion, at least in part, of the bacterial degradation member, and the bacterial degradation member prevents erosion, at least in part, of the drug component. In some embodiments, the bacterial degradation moiety is digestible by one or more microorganisms present in the colon of the individual. In some embodiments, the bacterial degradation moiety comprises one or more saccharides. In some embodiments, the one or more saccharides comprises any one or more of a sucrose, glucose, xylose, fructose, maltose, galactose, pectin, galactomannan, dextran, inulin, chitosan, carrageenan, cellulose acetate propionate (CAP), peptidoglycan, gellan, xanthan gum, lentinan, psyllium polysaccharide, maize bran arabinoxylan, alginate, hyaluronic acid, fucoidan, shellac, agar, or maltodextrin. In some embodiments, the bacterial degradation member is a layer, and wherein the layer has a thickness, as based on the direction of erosion, of about 0.1 mm to about 5 mm. In some embodiments, the bacterial degradation member comprises a surface area exposed to a gastrointestinal fluid in the individual of about 10 mm² to about 400 mm².

In some embodiments, the delay component does not comprise an erodible delay member. In some embodiments, the erodible material of the pH-based enteric member comprises one or more of a hypromellose acetate succinate, hydroxypropyl methyl cellulose phthalate, cellulose acetate propionate (CAP), poly (methacrylic acid-co-ethyl acrylate), or polyvinyl acetate phthalate (PVAP). In some embodiments, the hypromellose acetate succinate is HPMCAS LG/LF, HPMCAS MG/MF, or HPMCAS HG/LF. In some embodiments, the hydroxypropyl methyl cellulose phthalate is HPMCP HP-50 or HPMCP HP-55. In some embodiments, the poly (methacrylic acid-co-ethyl acrylate) is Eudragit L100-55 or Eudragit L100.

In some embodiments, the drug component comprises a top surface, a bottom surface, and one or more side surfaces, and wherein the drug component is embedded in the delay component such that erosion of the delay component, or an aspect thereof, exposes at least two surfaces of any of the top surface, the bottom surface, or at least one of the one or more side surfaces to gastrointestinal fluid of the individual. In some embodiments, the oral drug dosage form does not comprise a shell.

In some embodiments, the oral drug dosage form further comprises a shell not admixed with the drug, wherein the oral drug dosage form is configured such that the shell, or a portion thereof, and the delay member prevent erosion of the drug component. In some embodiments, the direction of erosion of the delay component and/or the drug component are based on the configuration of the shell.

In some embodiments, the oral drug dosage form further comprises a protease scavenger and/or a protease inhibitor. In some embodiments, the protease scavenger or the protease inhibitor is admixed with the delay component, or a portion thereof. In some embodiments, the protease scavenger or the protease inhibitor is admixed in the erodible material of the erodible delay member.

In some embodiments, the oral drug dosage form is configured to release the drug at, near, or downstream of any one of the cecum, ascending colon, hepatic flexure, transverse colon, splenic flexure, descending colon, or sigmoid colon. In some embodiments, at least 95% of the drug in the oral drug dosage form is released from the oral drug dosage form in the colon of the individual. In some embodiments, less than 2% of the drug in the oral drug dosage form is released from the oral drug dosage form outside of the colon of the individual. In some embodiments, all of the drug in the oral drug dosage form is released from the oral drug dosage form in the colon of the individual.

In some embodiments, the drug component and/or the delay component are in the form of one or more layers. In some embodiments, the layer of the pH-based enteric member comprises a top surface and a bottom surface, wherein the layer of the erodible delay member comprises a top surface and a bottom surface, wherein the layer of the drug component comprises a top surface and a bottom surface, wherein the bottom surface of the layer of the pH-based enteric member is in contact with the top surface of the layer of the erodible delay member, and wherein the bottom surface of the layer of the erodible delay member is in contact with the top surface of the layer of the drug component.

In some embodiments, the oral drug dosage form is configured to have more than one compartment, and wherein at least one compartment comprises the drug component and the delay component.

In some embodiments, the pH-based enteric member erodes at or above a pH value of about 5.5. In some embodiments, the pH-based enteric member is a layer having a thickness, as based on the direction of erosion, of about 0.1 mm to about 5 mm. In some embodiments, the pH-based enteric member comprises a surface area exposed to a gastrointestinal fluid in the individual of about 10 mm² to about 400 mm². In some embodiments, exterior surfaces of the oral drug dosage form, prior to administration to the individual, comprise the pH-based enteric member, and, optionally, the shell. In some embodiments, the pH-based enteric member comprises a thermoformable material. In some embodiments, the pH-based enteric member comprises one or more of: stearic acid, copolyvidone, polyvinylpyrrolidone-co-vinyl-acetate, polyvinylpyrrolidone-polyvinyl acetate copolymer, crospovidone, polyvinylpyrrolidone, hydroxypropyl cellulose, hydroxylpropyl methylcellulose or hypromellose, hydroxypropyl methylcellulose phthalate, methyl cellulose, methacrylic acid copolymer, poly (methacrylic acid-co-ethyl acrylate), poly (butyl methacrylate-co-(2-dimethylaminoethyl) methacrylate-co-methyl methacrylate), poly (dimethylaminoethylmethacrylate-co-methacrylic esters), poly (ethyl acrylate-co-methyl methacrylate-co-trimethylammonioethyl methacrylate chloride), poly (methyl acrylate-co-methyl methacrylate-co-methacrylic acid), poly (methacrylic acid-co-methylmethacrylate), poly (methacylic acid-co-ethyl acrylate), poly (methacylic acid-co-methyl methacrylate), polyethylene oxide, polyethylene glycol, polyvinyl caprolactam-polyvinyl acetate-polyethylene glycol graft copolymer, polyethylene glycol-polyvinyl alcohol graft copolymer, Kollicoat IR-polyvinyl alcohol, polyvinyl alcohol, aminoalkyl methacrylate copolymer E, hydroxypropyl methylcellulose acetate succinate or hypromellose acetate succinate, methacrylic ester copolymer, ammonioalkyl methacrylate copolymer, ethyl cellulose, polyvinyl acetate and polyvinylpyrrolidone, polyvinyl acetal diethyl aminolactate, polyvinyl acetal diethyl amino lactate, maltitol, lactose monohydrate, xylitol, isomalt, sucrose, glucose, dextran, or maltose.

In some embodiments, the pH-based enteric member erodes at or above a pH value of about 6.8. In some embodiments, the pH-based enteric member is a layer having a thickness, as based on the direction of erosion, of about 0.1 mm to about 5 mm. In some embodiments, the pH-based enteric member comprises a surface area exposed to a gastrointestinal fluid in the individual of about 10 mm² to about 400 mm². In some embodiments, exterior surfaces of the oral drug dosage form, prior to administration to the individual, comprise a pH-based enteric member, and, optionally, a shell. In some embodiments, the pH-based enteric member comprises a thermoformable material. In some embodiments, the pH-based enteric member comprises one or more of: poly (methacrylic acid, methylmethacrylate), poly (methacrylic acid, methyl methacrylate), methyl acrylate, methyl methacrylate and methacrylic acid. In some embodiments, the pH-based enteric member comprises one or more of: Eudragit L 100, Eudragit L 12.5, Eudragit L 12.5P, Eudragit L 100-55, Eudragit L 30D, Eudragit S 100, Eudragit S 12.5, Eudragit S 12.5P, Eudragit FS 30D. In some embodiments, the pH-based enteric member comprises one or more of: stearic acid, copolyvidone, polyvinylpyrrolidone-co-vinyl-acetate, polyvinylpyrrolidone-polyvinyl acetate copolymer, crospovidone, polyvinylpyrrolidone, hydroxypropyl cellulose, hydroxylpropyl methylcellulose or hypromellose, hydroxypropyl methylcellulose phthalate, methyl cellulose, methacrylic acid copolymer, poly (methacrylic acid-co-ethyl acrylate), poly (butyl methacrylate-co-(2-dimethylaminoethyl) methacrylate-co-methyl methacrylate), poly (dimethylaminoethylmethacrylate-co-methacrylic esters), poly (ethyl acrylate-co-methyl methacrylate-co-trimethylammonioethyl methacrylate chloride), poly (methyl acrylate-co-methyl methacrylate-co-methacrylic acid), poly (methacrylic acid-co-methylmethacrylate), poly (methacylic acid-co-ethyl acrylate), poly (methacylic acid-co-methyl methacrylate), polyethylene oxide, polyethylene glycol, polyvinyl caprolactam-polyvinyl acetate-polyethylene glycol graft copolymer, polyethylene glycol-polyvinyl alcohol graft copolymer, Kollicoat IR-polyvinyl alcohol, polyvinyl alcohol, aminoalkyl methacrylate copolymer E, hydroxypropyl methylcellulose acetate succinate or hypromellose acetate succinate, methacrylic ester copolymer, ammonioalkyl methacrylate copolymer, ethyl cellulose, polyvinyl acetate and polyvinylpyrrolidone, polyvinyl acetal diethyl aminolactate, polyvinyl acetal diethyl amino lactate, maltitol, lactose monohydrate, xylitol, isomalt, sucrose, glucose, dextran, or maltose.

In some embodiments, the erodible delay member prevents release of the drug from the drug component for at least about 10 minutes following contact of the erodible delay member to a gastrointestinal fluid in the individual. In some embodiments, the erodible delay member is a layer having a thickness, as based on the direction of erosion, of about 0.1 mm to about 5 mm. In some embodiments, the erodible delay member comprises a surface area exposed to a gastrointestinal fluid in the individual of about 10 mm² to about 400 mm². In some embodiments, the erodible delay member comprises a thermoformable material. In some embodiments, the erodible delay member comprises one or more of stearic acid, medium chain triglycerides, glyceryl distearate, propylene glycol monolaurate, propylene glycol caprylate, oleoyl polyoxyl-6 glycerides, PEG-6 STEARATE, PEG-32 STEARATE, linoleoyl polyoxyl-6 glycerides, lauroyl polyoxyl-32 glycerides, caprylocaproyl polyoxyl-8 glycerides, sorbitan monolaurate, sorbitan monopalmitate, sorbitan monostearate, sorbitan oleate, glyceryl monolinoleate, copolyvidone, polyvinylpyrrolidone-co-vinyl-acetate, polyvinylpyrrolidone-polyvinyl acetate copolymer, crospovidone, polyvinylpyrrolidone, hydroxypropyl cellulose, hydroxylpropyl methylcellulose or Hypromellose, hydroxypropyl methylcellulose phthalate, methyl cellulose, methacrylic acid copolymer, poly (methacrylic acid-co-ethyl acrylate), poly (butyl methacrylate-co-(2-dimethylaminoethyl) methacrylate-co-methyl methacrylate), poly (dimethylaminoethylmethacrylate-co-methacrylic esters), poly (ethyl acrylate-co-methyl methacrylate-co-trimethylammonioethyl methacrylate chloride), poly (methyl acrylate-co-methyl methacrylate-co-methacrylic acid), poly (methacrylic acid-co-methylmethacrylate), poly (methacylic acid-co-ethyl acrylate), poly (methacylic acid-co-methyl methacrylate), polyethylene oxide, polyethylene glycol, polyvinyl caprolactam-polyvinyl acetate-polyethylene glycol graft copolymer, polyethylene glycol-polyvinyl alcohol graft copolymer, Kollicoat IR-polyvinyl alcohol, polyvinyl alcohol, aminoalkyl methacrylate copolymer E, hydroxypropyl methylcellulose acetate succinate or hypromellose acetate succinate, methacrylic ester copolymer, ammonioalkyl methacrylate copolymer, ethyl cellulose, polyvinyl acetate and polyvinylpyrrolidone, polyvinyl acetal diethyl aminolactate, polyvinyl acetal diethyl amino lactate, maltitol, lactose monohydrate, xylitol, isomalt, sucrose, glucose, dextran, maltose, Lauroyl Polyoxyl-32 glycerides, caprylocaproyl polyoxyl-8 glycerides, polyoxyl (35) Castor oil, Vitamin E polyethylene glycol succinate, PEG-40 hydrogenated Castor oil, glyceryl monolinoleate, or glyceryl dibehenate.

In some embodiments, the drug component is not in direct contact with the pH-based enteric member. In some embodiments, the drug component is a controlled-release drug component. In some embodiments, the drug component is a sustained-release drug component. In some embodiments, the drug component is an immediate-release drug component. In some embodiments, the drug component is configured to release the drug from the oral drug dosage form for at least about 1 hour. In some embodiments, the drug component is configured to provide a drug release profile comprising a zero-order release profile, first-order release pro file, delayed release profile, pulsed release profile, iterative pulsed release profile, immediate release profile, or sustained release profile, or a combination thereof. In some embodiments, the release of the drug is based, at least in part, on an in vitro dissolution study. In some embodiments, the release of the drug is based, at least in part, on an in vivo dissolution study. In some embodiments, the drug component has a drug mass fraction (m_{F}) of the drug of about 0.05 to about 0.6. In some embodiments, the weight ratio of the drug component to the delay component is about 1:10 to about 10:1. In some embodiments, the drug component is a layer having a thickness, as based on the direction of erosion, of about 0.1 mm to about 5 mm. In some embodiments, the drug component comprises a surface area exposed to a gastrointestinal fluid in the individual of about 10 mm² to about 400 mm². In some embodiments, the drug component comprises a thermoformable material. In some embodiments, the drug component comprises one or more of copolyvidone, polyvinylpyrrolidone-co-vinyl-acetate, polyvinylpyrrolidone-polyvinyl acetate copolymer, polyvinylpyrrolidone, hydroxypropyl cellulose, hydroxylpropyl methylcellulose or Hypromellose, polyethylene oxide, polyethylene glycol, polyvinyl caprolactam-polyvinyl acetate-polyethylene glycol graft copolymer, polyethylene glycol-polyvinyl alcohol graft copolymer, Kollicoat IR-polyvinyl alcohol, polyvinyl alcohol, aminoalkyl methacrylate copolymer E, hydroxypropyl methylcellulose acetate succinate hypromellose acetate succinate, hydroxypropyl methylcellulose phthalate, maltitol, lactose monohydrate, xylitol, isomalt, sucrose, glucose, or dextran 70. In some embodiments, the oral drug dosage form further comprises one to four additional drug components.

In some embodiments, the drug is an anti-inflammatory agent, non-steroidal anti-inflammatory agent, steroid, immunosuppressant, antibiotic, biological agent, anti-neoplastic agent, analgesic, anaesthetic, anticonvulsant, antidiabetic agent, antihistamine, anti-infective, antineoplastic, antiparkinsonian agent, antirheumatic agent, appetite stimulant, appetite suppressant, blood modifier, bone metabolism modifier, cardiovascular agent, central nervous system depressant, central nervous system stimulant, decongestant, dopamine receptor agonist, electrolyte, gastrointestinal agent, immunomodulator, muscle relaxant, narcotic, parasympathomimetic, sympathomimetic, sedative, hypnotic, or vaccine. In some embodiments, the anti-inflammatory agent is a JAK inhibitor. In some embodiments, the JAK inhibitor is tofacitinib or a pharmaceutically acceptable salt thereof. In some embodiments, the anti-inflammatory agent is mesalazine, sulphasalazine, or balsalazide. In some embodiments, the non-steroidal anti-inflammatory agent is ibuprofen or diclofenac. In some embodiments, the steroid is prednisolone, budesonide, or fluticasone. In some embodiments, the immunosuppressant is azathioprine, cyclosporin, or methotrexate. In some embodiments, the biological agent is a peptide, protein, antibody, or a fragment thereof, or nucleic acid. In some embodiments, the antibody is infliximab, adalimumab, certolizumab pegol, golimumab, or ustekinumab. In some embodiments, the antineoplastic agent is fluorouracil, methotrexate, dactinomycin, bleomycin, etoposide, taxol, vincristine, doxorubicin, cisplatin, daunorubicin, Etoposide, raltitrexed, or oxaliplatin, or a combination thereof. In some embodiments, the oral drug dosage form comprises about 0.01 mg to about 500 mg of the drug.

In some embodiments, the oral drug dosage form further comprises an excipient. In some embodiments, the excipient comprises one or more of an absorption enhancer, pH-lowering agent, or a disintegrant. In some embodiments, the excipient is admixed in the drug component.

In some embodiments, the shell comprises an insulating material that is impermeable to bodily fluids. In some embodiments, the insulating material comprises one or more of an ethylcellulose (EC), dibutyl sebacate (DBS), and titanium dioxide. In some embodiments, the insulating material is a non-erodible material. In some embodiments, the insulating material is an erodible material having a pH-based erosion and/or an erosion rate that allows for the complete release of the drug from the oral drug dosage form prior to exposure of the drug component to bodily flu ids due to erosion of the shell.

In another aspect, provided herein is a commercial batch of any oral drug dosage form of described herein, wherein the commercial batch has a standard deviation of about 0.05 or less for each of the following: an amount of a drug in the oral drug dosage form; a weight of the oral drug dosage form; a largest crossing dimension of the oral drug dosage form; and a crossing dimension perpendicular to the largest crossing dimension of the oral drug dosage form. In some embodiments, the commercial batch comprises at least about 1000 of the oral drug dosage forms.

In another aspect, provided herein is a method of three-dimensional (3D) printing of any oral drug dosage form described herein, the method comprising dispensing materials according to a layer-by-layer model of the oral drug dosage form to print the oral drug dosage form, wherein each layer of the layer-by-layer model is printed by dispensing, as necessary, for a layer: (a) a shell material to form any portion of a shell in the layer; (b) a drug component material to form any portion of a drug component in the layer; (c) a pH-based enteric member material to form any portion of a pH-based enteric member in the layer; (d) an erodible delay member material to form any portion of an erodible delay member in the layer; (e) a bacterial degradation member material to form any portion of a bacterial degradation member in the layer; and (f) an excipient component material to form any portion of an excipient component in the layer. In some embodiments, the method further comprises generating the layer-by-layer model of the oral drug dosage form. In some embodiments, the dispensing is via melt extrusion deposition (MED). In some embodiments, dispensing of the shell material, dispensing of the drug component material, dispensing of the pH-based enteric member material, dispensing of the erodible delay member material, and dispensing of the shell material are each performed by a different printing head. The melt extrusion deposition (MED) is a 3D printing method, such as described in WO2021042865A1, WO2021164660A1, WO2022089588A1, WO2022089631A1, which are hereby incorporated herein by references in their entirety.

In another aspect, provided herein is a method of treating a condition in an individual, the method comprising administering to the individual any oral drug dosage form described herein. In some embodiments, the condition is selected from the group consisting of a gastrointestinal disorder, central nervous system (CNS) disorder, cardiovascular disease, hypertension, atherosclerosis, angina, arterial obstruction, peripheral arterial disease, myocardial pathology, arrhythmia, acute myocardial infarction, angina, cardiomyopathy, congestive heart failure, coronary artery disease (CAD), carotid artery disease, endocarditis, hypercholesterolemia, hyperlipidemia, peripheral artery disease (PAD), genitourinary disorders; erectile dysfunction, urinary organ diseases benign prostatic hypertrophy (BPH), renal tubular acidosis, diabetic nephropathy, glomerulonephritis, glomerulosclerosis, urinary tract infection, faecal incontinence, ocular disease glaucoma, blepharitis, ocular hypertension, retinopathy, conjunctivitis, scleritis, retinitis, keratitis, corneal ulcer, iritis, chorioretinal inflammation, macular edema, xerophthalmia, pulmonary disease asthma, pulmonary hypertension, acute respiratory distress syndrome, COPD, emphysema, pneumonia, tuberculosis, bronchitis, acute bronchitis, bronchiectasis, bronchiolitis, bronchopulmonary dysplasia, byssinosis, coccidioidomycosis (Cocci), cystic fibrosis, influenza, lung cancer, mesothelioma, metabolic disease, hypercalciuria, hyperglycemia, hyperinsulinemic hypoglycemia, hyperinsulinism, hyperlysinuria, and hypoglycemia. In some embodiments, the gastrointestinal disorder is selected from the group consisting of irritable bowel disease (IBD), irritable bowel syndrome (IBS), constipation, diarrhea, infection, and carcinoma. In some embodiments, the IBD is associated with Crohn's disease or ulcerative colitis. In some embodiments, the carcinoma is colon cancer or colorectal cancer. In some embodiments, the CNS disorder is selected from the group consisting of neurogenic pain, stroke, dementia, Alzheimer's disease, Parkinson's disease, neuronal degeneration, meningitis, spinal cord injury, cerebral vasospasm, and amyotrophic lateral sclerosis.

In another aspect, provided herein is a method of providing local delivery of a drug to the colon of an individual, the method comprising administering to the individual any oral drug dosage form described herein, wherein the individual is suffering from a gastrointestinal disorder.

In another aspect, provided herein is a method of providing systemic delivery of a drug to an individual, the method comprising administering to the individual any oral drug dosage form described herein, wherein the individual is suffering from a central nervous system (CNS) disorder, cardiovascular disease, hypertension, atherosclerosis, angina, arterial obstruction, peripheral arterial disease, myocardial pathology, arrhythmia, acute myocardial infarction, angina, cardiomyopathy, congestive heart failure, coronary artery disease (CAD), carotid artery disease, endocarditis, hypercholesterolemia, hyperlipidemia, peripheral artery disease (PAD), genitourinary disorders; erectile dysfunction, urinary organ diseases benign prostatic hypertrophy (BPH), renal tubular acidosis, diabetic nephropathy, glomerulonephritis, glomerulosclerosis, urinary tract infection, faecal incontinence, ocular disease glaucoma, blepharitis, ocular hypertension, retinopathy, conjunctivitis, scleritis, retinitis, keratitis, corneal ulcer, iritis, chorioretinal inflammation, macular edema, xerophthalmia, Pulmonary disease asthma, pulmonary hypertension, acute respiratory distress syndrome, COPD, emphysema, pneumonia, tuberculosis, bronchitis, acute bronchitis, bronchiectasis, bronchiolitis, bronchopulmonary dysplasia, byssinosis, coccidioidomycosis (Cocci), cystic fibrosis, influenza, lung cancer, mesothelioma, metabolic disease, hypercalciuria, hyperglycemia, hyperinsulinemic hypoglycemia, hyperinsulinism, hyperlysinuria, or hypoglycemia.

In some embodiments, the methods of designing provided herein comprising using one or more tracking stripes such that external imagine techniques, e.g., x-ray imaging, can be used to assess the status (such as not yet undergoing erosion/release from the oral drug dosage form, undergoing erosion/release from the oral drug dosage form, or completion of erosion/release from the oral drug dosage form) of one or more components of an oral drug dosage form taught herein relative to the location of the oral drug dosage form in the individual to which it was administered. For example, such tracking features then enable one to confirm that one or more components on an oral drug dosage form are behaving as desired, such as release of a drug from a drug component is occurring at a desired location in the gastrointestinal tract of an individual. This further enables methods comprising adjusting the design of an oral drug dosage form to provide the desired results. For instances, if it is found that a drug component is eroding and releasing drug in a location earlier than desired (e.g., upstream of the desired location in the gastrointestinal tract), then one or more aspects of the delay component can be adjusted, such as by changing a composition or thickness of a material thereof.

In some embodiments, provided is a method of designing an oral drug dosage form configured to release a drug at a desired gastrointestinal location in an individual, wherein the oral drug dosage form comprises: a drug component comprising: a first erodible material admixed with a drug, and a first erodible tracking stripe; and a shell comprising a second tracking stripe; a delay component not admixed with the drug, the delay component comprising: a pH-based enteric member configured to erode at or above a pre-determined pH value, wherein the pH based enteric member comprises a third erodible tracking stripe; and an erodible delay member comprising a second erodible material, wherein the erodible delay member comprises a fourth erodible tracking stripe, wherein the pH-based enteric member, alone or in combination with the shell, prevents erosion of the erodible delay member, and wherein the erodible delay member, alone or in combination with the shell, prevents erosion of the drug component, the method comprising: (a) administering the oral drug dosage form to an individual; (b) imaging the individual over a time course to obtain a location of the oral drug dosage form and a status of the first erodible tracking stripe, the second erodible tracking stripe, the third erodible tracking stripe, and the fourth tracking stripe; and (c) adjusting the drug component and/or delay component and/or shell, or portion thereof, based on the location and the status of the first erodible tracking stripe, the second erodible tracking stripe, the third erodible tracking stripe, and the fourth track stripe to design the oral drug dosage form configured to release a drug at a desired gastrointestinal location in an individual.

In some embodiments, the adjusting the delay component comprises adjusting the pH-based enteric member by changing one or more of the composition, sur face area exposed to bodily fluid following administration, or thickness of the pH-based enteric member. In some embodiments, the adjusting the delay component comprises adjusting the erodible delay member by changing one or more of the composition, surface area exposed to bodily fluid following administration, or thickness of the erodible delay member.

In some embodiments, provided herein is a method of designing an oral drug dosage form configured to release a drug at a desired gastrointestinal location in an individual, wherein the oral drug dosage form comprises: a drug component comprising: a first erodible material admixed with a drug, and a first erodible tracking stripe; and a shell; a delay component not admixed with the drug, the delay component comprising: a pH-based enteric member configured to erode at or above a pre-determined pH value; and an erodible delay member comprising a second erodible material, wherein the pH-based enteric member, alone or in combination with the shell, prevents erosion of the erodible delay member, and wherein the erodible delay member, alone or in combination with the shell, prevents erosion of the drug component, the method comprising: (a) administering the oral drug dosage form to an individual; (b) imaging the individual over a time course to obtain a location of the oral drug dosage form and a status of the first erodible tracking stripe; and (c) adjusting the drug component and/or delay component and/or shell, or portion thereof, based on the location and the status of the first erodible tracking stripe, to design the oral drug dosage form configured to release a drug at a desired gastrointestinal location in an individual.

In some embodiments, the first erodible material admixed with the drug and the first erodible tracking stripe are not in direct contact with one another. In some embodiments, the shell comprises a second tracking stripe, and wherein the second tracking stripe is configured to be imaged by the imaging such as to obtain a status of the second tracking stripe. In some embodiments, the adjusting is based on the location and the status of the first erodible tracking stripe and/or the second tracking stripe. In some embodiments, the pH-based enteric member comprises a third tracking stripe, and wherein the third tracking stipe is configured to be imaged by the imaging such as to obtain a status of the third erodible tracking stripe. In some embodiments, the adjusting is based on the location and the status of the first erodible tracking stripe and/or the second tracking stripe and/or the third erodible tracking stripe. In some embodiments, the erodible delay member comprises a fourth tracking stripe, and wherein the fourth tracking stripe is configured to be imaged by the imaging such as to obtain a status of the fourth erodible tracking stripe. In some embodiments, the adjusting is based on the location and the status of the first erodible tracking stripe and/or the second tracking stripe and/or the third erodible tracking stripe and/or the fourth erodible tracking stripe.

In some embodiments, the imaging provides a location of the oral drug dosage form when the first erodible tracking stripe begins to erode. In some embodiments, the imaging provides a location of the oral drug dosage form when the first erodible tracking stripe has completely eroded. In some embodiments, the adjusting comprises adjusting the delay component, or portion thereof, to increase the delay of release of the drug from the oral drug dosage form based on the location and the status of the first erodible tracking stripe showing earlier than desired release. In some embodiments, the adjusting comprises adjusting the delay component, or portion thereof, to decrease the delay of release of the drug from the oral drug dosage form based on the location and the status of the first erodible tracking stripe showing later than desired release.

In some embodiments, the first tracking stripe comprises barium sulfate. In some embodiments, the imagining comprises x-ray imaging. In some embodiments, the method further comprises obtaining one or more pharmacokinetic (PK) parameters associated with the drug following the administration of the oral drug dosage form to an individual. In some embodiments, the method further comprises identifying a relationship between the one or more PK parameters and the locational and status information of the first tracking stripe. In some embodiments, the method further comprises adjusting the drug component and/or delay component, or portion thereof, based on the relationship between the one or more PK parameters and the locational and status information of the first tracking stripe. In some embodiments, the imaging does not involve a magnetic-based technique or an invasive imaging technique.

In some embodiments, provided herein is a method of designing an oral drug dosage form configured to release a drug at a desired gastrointestinal location in an individual, wherein the oral drug dosage form comprises: a drug component comprising a first erodible material admixed with a drug; a shell; and a delay component not admixed with the drug, the delay component comprising: a pH-based enteric member configured to erode at or above a pre-determined pH value; and an erodible delay member comprising a second erodible material, wherein the pH-based enteric member prevents erosion of the erodible delay member, and wherein the erodible delay member prevents erosion of the drug component, wherein the oral drug dosage form comprises at least one tracking stripe positioned in a component thereof, the method comprising: (a) administering the oral drug dosage form to an individual; (b) imaging the individual over a time course to obtain a location of the oral drug dosage form and a status of the at least one tracking stripe; and (c) adjusting the drug component and/or delay component and/or shell, or portion thereof, based on the location and the status of the at least one tracking stripe, to design the oral drug dosage form configured to release a drug at a desired gastrointestinal location in an individual.

In some embodiments, one of the tracking stripes is located in, or near, the drug component. In some embodiments, the tracking stripes is located in the shell. In some embodiments, one of the tracking stripes is located in the pH-based enteric member. In some embodiments, one of the tracking stripes is located in the erodible delay member. In some embodiments, at least one of the at least one tracking stripes is erodible.

In some embodiments, provided herein is a method of designing an oral drug dosage form configured to release a drug at a desired gastrointestinal location in an individual, wherein the oral drug dosage form comprises: a drug component comprising a first erodible material admixed with a drug; and a delay component not admixed with the drug, the delay component comprising: a pH-based enteric member configured to erode at or above a pre-determined pH value; and an erodible delay member comprising a second erodible material, wherein the pH-based enteric member prevents erosion of the erodible delay member, and wherein the erodible delay member prevents erosion of the drug component, wherein the oral drug dosage form comprises at least one tracking stripe positioned in a component thereof, the method comprising: (a) administering the oral drug dosage form to an individual; (b) imaging the individual over a time course to obtain a location of the oral drug dosage form and a status of the at least one tracking stripe; and (c) adjusting the drug component and/or delay component, or portion thereof, based on the location and the status of the at least one tracking stripe, to design the oral drug dosage form configured to release a drug at a desired gastrointestinal location in an individual.

In some embodiments, one of the tracking stripes is located in, or near, the drug component. In some embodiments, one of the tracking stripes is located in the pH-based enteric member. In some embodiments, one of the tracking stripes is located in the erodible delay member. In some embodiments, at least one of the at least one tracking stripes is erodible.

In some aspects, provided herein is a method for treating ulcerative colitis, the method comprising administering to a human individual an oral drug dosage form of any one of claims 1-84, wherein the oral drug dosage form comprises a JAK inhibitor. In some embodiments, the JAK inhibitor comprises tofacitinib. In some embodiments, the oral drug dosage form comprises about 11 mg or less of tofacitinib. In some embodiments, the oral drug dosage form comprises about 5.5 mg or less of tofacitinib. In some embodiments, the oral drug dosage form is administered once daily.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

All references cited herein, including patent applications and publications, are incorporated by reference in their entirety.

### BRIEF DESCRIPTION OF THE DRAWINGS

FIGS. 1A-1K show cross-sectional diagrams of example oral drug dosage forms provided herein.

FIG. 2 shows an example oral drug dosage form provided herein including various cross-sectional diagrams thereof.

FIG. 3 shows an example oral drug dosage form provided herein including various cross-sectional diagrams thereof.

FIG. 4 shows an example oral drug dosage form provided herein including various cross-sectional diagrams thereof.

FIG. 5 shows an in vitro dissolution profile of an example oral drug dosage form provided herein (see Tables 1A and 1B, and FIG. 2) with an overlay of the erosion of certain components therein including a set of tracking stripes. For each time point of the dissolution profile, the data point is based on the mean of four measurements and the standard deviation is provided using error bars. The delay time of the oral drug dosage form in vitro is about 4.5 hours.

FIG. 6A shows an in vitro dissolution profile of an example oral drug dosage form provided herein (see Tables 2A and 2B, and FIG. 3) with an overlay of the erosion of certain components therein including a set of tracking stripes. For each time point of the dissolution profile, the data point is based on the mean of three measurements and the standard deviation is provided using error bars. The delay time of the oral drug dosage form in vitro is about 4 hours. FIG. 6B shows photos taken during the dissolution process.

FIG. 7 shows an in vitro dissolution profile of an example oral drug dosage form provided herein (see Tables 3A and 3B, and FIG. 4). For each time point of the dissolution profile, the data point is based on the mean of three measurements and the standard deviation is provided using error bars. The delay time of the oral drug dosage form in vitro is about 4.5 hours.

FIG. 8 shows an in vitro dissolution profile of an example oral drug dosage form provided herein (see Tables 4A and 4B, and FIG. 4) with an overlay of the erosion of certain components therein including a set of tracking stripes. For each time point of the dissolution profile, the data point is based on the mean of three measurements and the standard deviation is provided using error bars. The delay time of the oral drug dosage form in vitro is about 5 hours.

FIGS. 9A and 9B show pharmacokinetic (PK) plots of a reference (Xeljanz XR) and example oral drug dosage forms provided herein.

FIGS. 10A-10C show X-ray image time courses of beagle dogs following administration of example oral drug dosage forms provided herein. Annotations are provided to point out the oral drug dosage form and to provide status information, such as drug release initiation and drug release completion.

FIG. 11 shows an example oral drug dosage form provided herein including various cross-sectional diagrams thereof.

FIGS. 12A-12D show in vitro dissolution profiles of example oral drug dosage forms provided herein (see Tables 5A-8B, and FIG. 11). For each time point of the dissolution profile, the data point is based on the mean of five measurements and the standard deviation is provided using error bars.

FIG. 13 shows pharmacokinetic (PK) plots of a reference (Xeljanz XR) and example oral drug dosage forms provided herein.

FIGS. 14A-14B show X-ray image time courses of beagle dogs following administration of example oral drug dosage forms provided herein. Annotations are provided to point out the oral drug dosage form and to provide status information, such as drug release initiation and drug release completion.

FIG. 15 shows pharmacokinetic (PK) plots measured from healthy volunteers of a reference (Xeljanz XR) and example oral drug dosage forms provided herein.

FIG. 16 shows X-ray image time courses of healthy volunteers following administration of example oral drug dosage forms provided herein. Annotations are provided to point out the oral drug dosage form and to provide status information, such as drug release initiation and drug release completion.

### DETAILED DESCRIPTION

Provided herein, in some aspects, is an oral drug dosage form configured to release a drug at a desired location in the colon of an individual, the oral drug dosage form comprising: a drug component comprising an erodible material admixed with the drug; and a delay component not admixed with the drug, wherein the delay component is configured to prevent release of the drug from the drug component until the oral drug dosage form reaches the colon of the in dividual following administration, and wherein the delay component comprises: a pH-based enteric member comprising an erodible material configured to erode at or above a pre-determined pH value. In some embodiments, the oral drug dosage form is configured such that substantially all, e.g., 100%, of the drug in the oral drug dosage form is released in the colon of an individual.

The oral drug dosage forms described herein are based on the inventors' unique insights and findings about the design and production of oral drug dosage forms for precise locational drug delivery in the colon of the gastrointestinal tract. As taught and demonstrated herein, in certain aspects, the oral drug dosage forms are capable of complete colon delivery of a drug (including complete delivery within a specific area of the colon), which enables maximization of therapeutic effectiveness of certain drugs while minimizing side effects caused by releasing the drug in another area of the gastrointestinal tract. For example, such toxicity can occur when undesired systemic absorption occurs due to a drug being release in another area of the gastrointestinal tract. The colon is susceptible to a number of disease states, including inflammatory bowel disease, irritable bowel syndrome, constipation, diarrhea, infection and carcinoma. The oral drug dosage forms taught herein, in certain aspects, are capable of effectively delivering a drug to the site of a disease state, namely, the colon, to provide effective local therapy. Additionally, in other aspects, the colon is a site of the gastrointestinal tract that provides the potential for effective uptake and thus systemic delivery. For example, the colon has comparably lower levels of several luminal and mucosal metabolic enzymes (e.g., proteases) and transporters, which presents therapeutic advantages for certain drugs that would otherwise not be absorbed into systemic circulation if released in another part of the gastrointestinal tract. For example, the lower proteolytic activity in the colon can be beneficial for the delivery of biologics (e.g., peptides, proteins, monoclonal antibodies, nucleic acids, and vaccines). The ability of the oral drug dosage forms taught herein to effect complete colon delivery (including delivery to a specific area of the colon) is a significant advancement in the field of drug delivery for both local and systemic therapeutics.

An illustrative example of the utility of certain oral drug dosage forms provided herein can be found in oral drug dosage forms described herein that are configured with a drug for treating ulcerative colitis. Ulcerative colitis is a condition that affects a specific area of the gastrointestinal tract, i.e., ulcerative colitis causes inflammation and ulcers of the innermost lining of the large intestine (colon) and rectum. Known agents, such as anti-inflammatory drugs including JAK inhibitors, are effective in treating an individual with ulcerative colitis by reducing inflammation and formation of ulcers. However, conventional drug dosage forms comprising such drugs have demonstrated shortcomings, such as incomplete drug delivery to the local site in need. This delivery outside of the colon reduces the effectiveness of such conventional oral drug dosage forms and can lead to systemic toxicity. The ad vantages of the oral drug dosage forms described herein provide a significant improvement in the treatment of such conditions, such as ulcerative colitis.

Conventionally, oral delivery of biologics, such as peptides, proteins, monoclonal antibodies and nucleic acids (e.g., RNA), is limited due to, at least in part, the presence of digestive enzymes in the gastrointestinal tract. Vaccines may also face high levels of degradation due to levels of phagocytosis. The lower level of functional proteolytic enzymes, functional digestive pancreatic enzymes, and phagocytosis in the colon, as compared to other areas of the gastrointestinal tract, presents an opportunity if such biologics can be safely delivered to the colon. In certain aspects, the oral drug dosage forms taught herein can protect a biological drug until release in the colon, thereby providing a significant advancement in the oral delivery of such therapeutic agents.

Thus, in certain aspects, provided herein is an oral drug dosage form configured to release a drug at a desired location in the colon of an individual, the oral drug dosage form comprising: a drug component comprising an erodible material admixed with the drug; and a delay component not admixed with the drug, wherein the delay component is configured to prevent release of the drug from the drug component until the oral drug dosage form reaches the colon of the individual following administration, wherein the delay component comprises: a pH-based enteric member comprising an erodible material configured to erode at or above a pre-determined pH value. In some embodiments, the delay component further comprises an erodible delay member, wherein the oral drug dosage form is configured such that the pH-based enteric member erodes prior to the erodible delay member. In some embodiments, the oral drug dosage form further comprises a second drug. In some embodiments, the oral drug dosage form further comprises a bacterial degradation moiety. In some embodiments, the delay component further comprises a bacterial degradation member comprising an erodible material admixed with the bacterial degradation moiety. In some embodiments, the delay component does not comprise an erodible delay member. In some embodiments, the oral drug dosage form does not comprise a shell. In some embodiments, the oral drug dosage form further comprises a shell not admixed with the drug, wherein the oral drug dosage form is configured such that the shell, or a portion thereof, and the delay member prevent erosion of the drug component.

In other aspects, provided herein is a commercial batch of any oral drug dosage form described herein, wherein the commercial batch has a standard deviation of about 0.05 or less for each of the following: an amount of a drug in the oral drug dosage form; a weight of the oral drug dosage form; a largest crossing dimension of the oral drug dosage form; and a crossing dimension perpendicular to the largest crossing dimension of the oral drug dosage form. In some embodiments, the commercial batch comprises at least about 1000 of the oral drug dosage forms.

In other aspects, provided herein is a method of three-dimensional (3D) printing of any oral drug dosage form described herein, the method comprising dispensing materials according to a layer-by-layer model of the oral drug dosage form to print the oral drug dosage form, wherein each layer of the layer-by-layer model is printed by dispensing, as necessary, for a layer: (a) a shell material to form any portion of a shell in the layer; (b) a drug component material to form any portion of a drug component in the layer; (c) a pH-based enteric member material to form any portion of a pH-based enteric member in the layer; (d) an erodible delay member material to form any portion of an erodible delay member in the layer; (e) a bacterial degradation member material to form any portion of a bacterial degradation member in the layer; and (f) an excipient component material to form any portion of an excipient component in the layer.

In other aspects, provided herein is a method of treating a condition in an individua 1, the method comprising administering to the individual an oral drug dosage form described herein.

In other aspects, provided herein is a method of providing local delivery of a drug to the colon of an individual, the method comprising administering to the individual an oral drug dosage form described herein.

In other aspects, provided herein is a method of providing systemic delivery of a drug to an individual, the method comprising administering to the individual an oral drug dosage form described herein.

### I. Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “peptide” and “protein” as used herein, may be used interchangeably to refer to a polymer comprising amino acid residues, and are not limited to a minimum length. Such polymers may contain natural or non-natural amino acid residues, or combinations thereof, and include, but are not limited to, peptides, oligopeptides, dimers, trimers, and multimers of amino acid residues. Full-length peptides or proteins, and fragments thereof, are encompassed by this definition. The terms also include modified species thereof, e.g., post-translational modifications of one or more residues, for example, methylation, phosphorylation glycosylation, sialylation, or acetylation.

As used herein, the term “tofacitinib” includes, unless otherwise indicated, any pharmaceutically acceptable form and salts thereof. In some embodiments, tofacitinib may be present in crystalline form. In some embodiments, tofacitinib may be present in amorphous form. In some embodiments, the pharmaceutically acceptable form is any pharmaceutically acceptable form, including, solvates, hydrates, isomorphs polymorphs, co-crystals, pseudomorphs, neutral forms, acid addition salt forms, and prodrugs. In some embodiments, the pharmaceutically acceptable form is a pharmaceutically acceptable salt. Conventional concentration and recrystallization techniques may be employed in generating and isolating pharmaceutically acceptable salts of a JAK inhibitor, including use of acids such as acetic acid, lactic acid, succinic acid, maleic acid, tartaric acid, citric acid, gluconic acid, ascorbic acid, mesylic acid, tosylic acid, benzoic acid, cinnamic acid, fumaric acid, sulfuric acid, phosphoric acid, hydrochloric acid, hydrobromic acid, hydroiodic acid, sulfamic acid, sulfonic acid, such as methanesulfonic, benzenesulfonic, and related acids. In some embodiments, tofacitinib is tofacitinib citrate.

As used herein, use of the terms “treat,” “treatment,” “treating,” or equivalents thereof, refer to an approach for obtaining beneficial or desired results including a reduction of symptoms of, e.g., a disease. For purposes of this disclosure, beneficial or desired clinical results include, but are not limited to, one or more of the following: alleviating one or more symptoms resulting from the disease, reducing the se verity of one or more symptoms resulting from the disease, preventing the increase in the severity of one or more symptoms resulting from the disease, decreasing the dose of one or more other medications required to treat and/or manage the disease, and in creasing the quality of life.

As used herein, the term “individual” refers to a mammal and includes, but is not limited to, human, bovine, horse, feline, canine, rodent, rat, mouse, dog, or primate. In some embodiments, the individual is a human individual.

The terms “comprising,” “having,” “containing,” and “including,” and other similar forms, and grammatical equivalents thereof, as used herein, are in tended to be equivalent in meaning and to be open ended in that an item or items foll owing any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

As used herein, the term “semisolid” and other similar forms, and grammatical equivalents thereof, refer to a viscous liquid that can flow under external forces (pressure, thrust, rotational force, gravity, etc.) or under process conditions (temperature, pressure, etc.).

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are al so included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Those skilled in the art will recognize that several embodiments are possible within the scope and spirit of the present disclosure. The following description illustrates the disclosure and, of course, should not be construed in any way as limiting the scope of the inventions described herein.

### II. Oral Drug Dosage Forms

Provided herein, in some aspects, is an oral drug dosage form configured to release a drug at a desired location in the colon. As described herein, in some embodiments, the oral drug dosage form configured to release a drug at a desired location in the colon of an individual, comprises: a drug component comprising an erodible material admixed with the drug; and a delay component not admixed with the drug, wherein the delay component is configured to prevent release of the drug from the drug component until the oral drug dosage form reaches the colon of the individual following administration, and wherein the delay component comprises: a pH-based enteric member comprising an erodible material configured to erode at or above a pre-determined pH value. In some embodiments, the oral drug dosage form has one or more additional features, e.g., an erodible delay member, a second drug and, optionally, a second drug component, a bacterial degradation moiety and, optionally, a bacterial degradation member, a shell, or an excipient. The oral drug dosage forms disclosed herein may comprise a variety of combinations of the components described herein, and may be arranged in a diverse array of configurations. In some instances, such as provided in section below, components and configurations are described in a modular fashion, and such description is not intended to limit the scope of the oral drug dosage forms encompassed herein.

### A. Example Oral Drug Dosage Forms

For purposes of illustration and explanation of the subject matter provided herein, certain oral drug dosage forms encompassed herein are described in detail below.

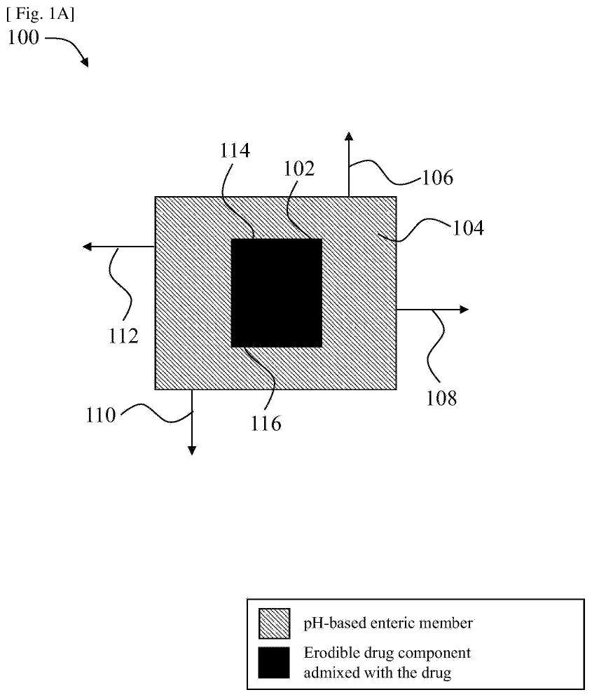

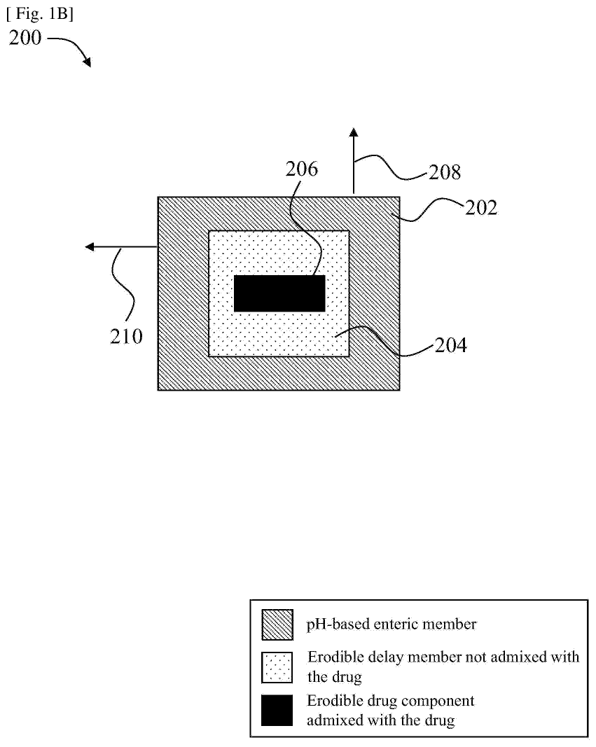

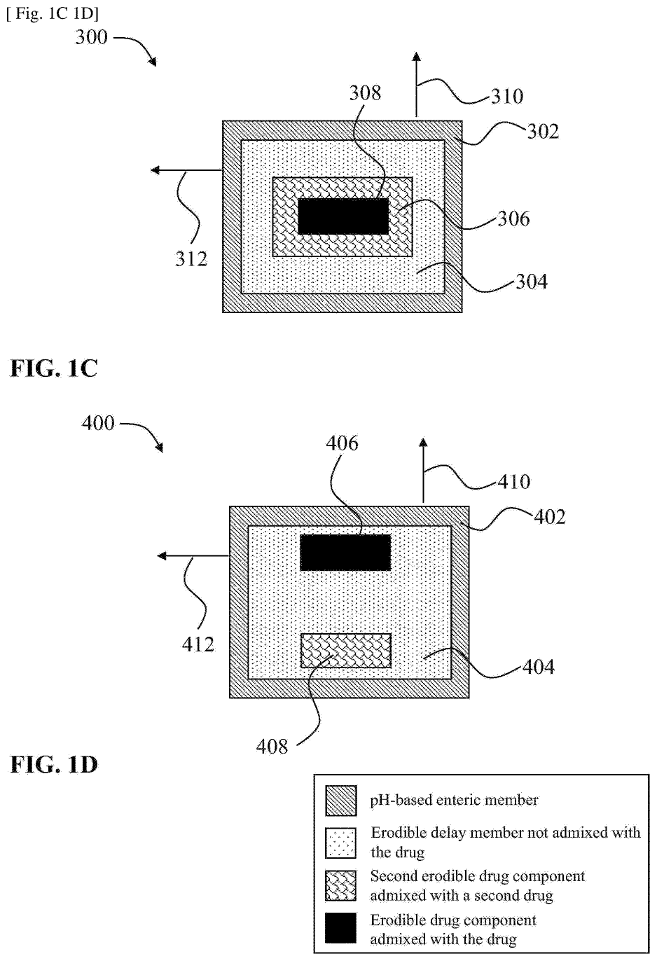

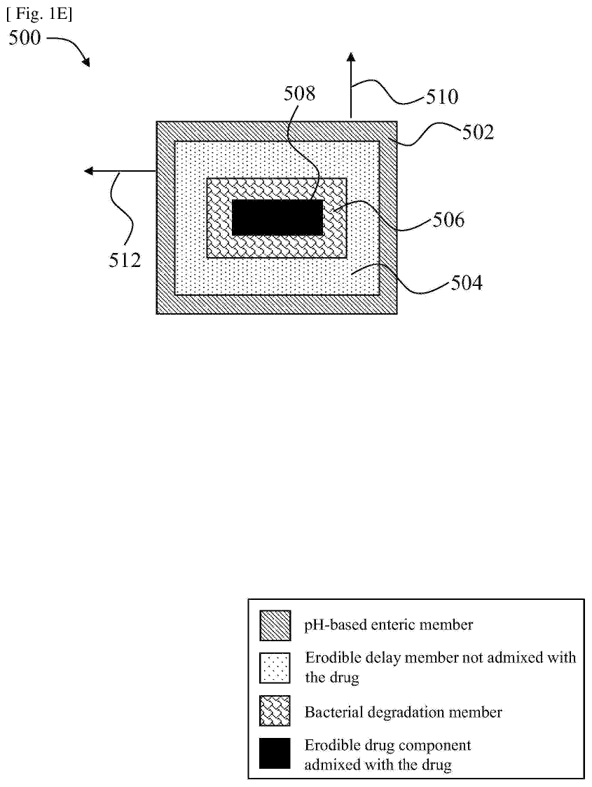

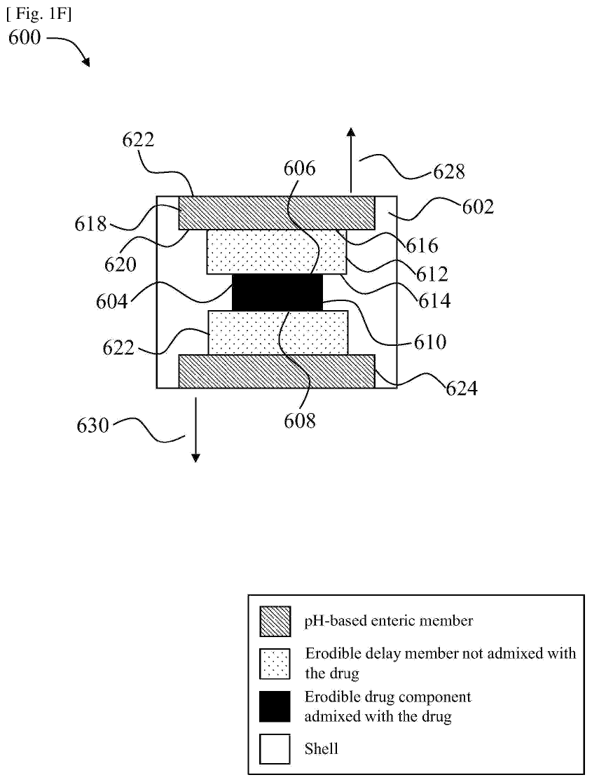

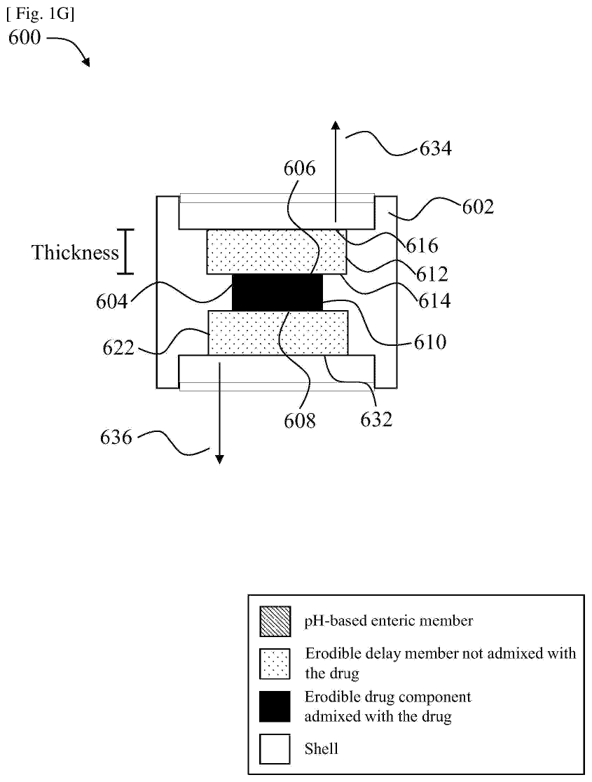

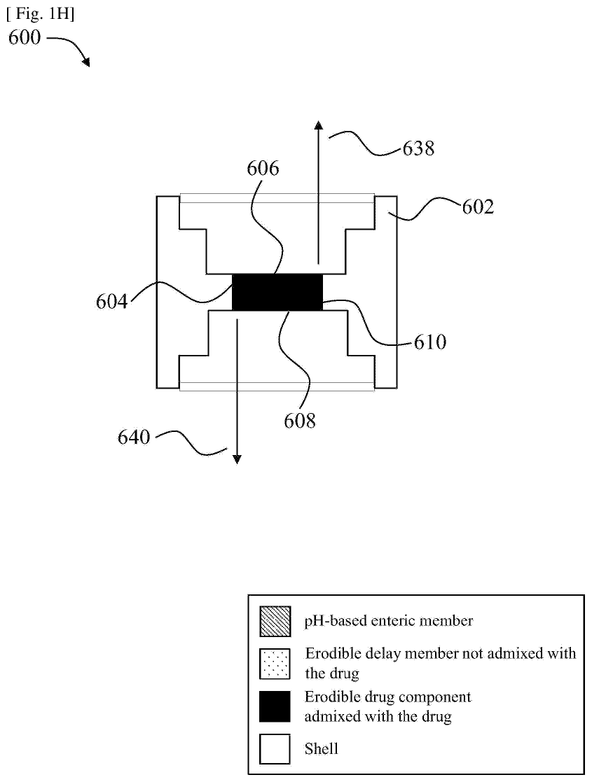

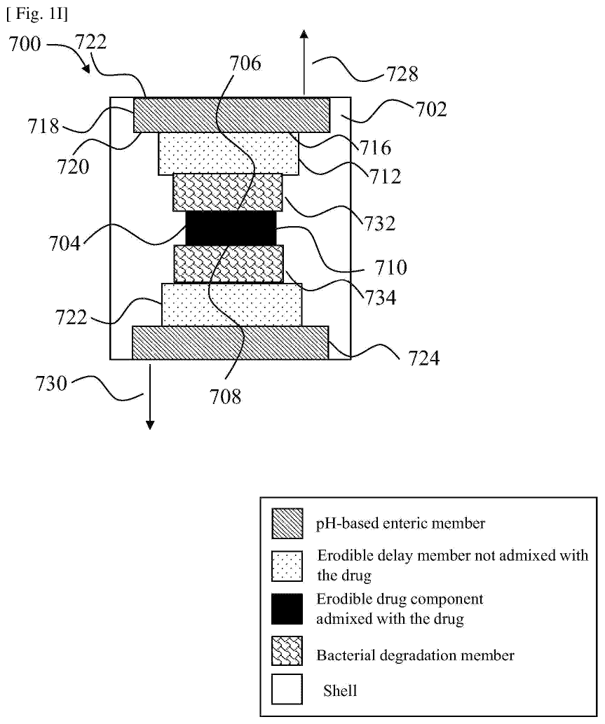

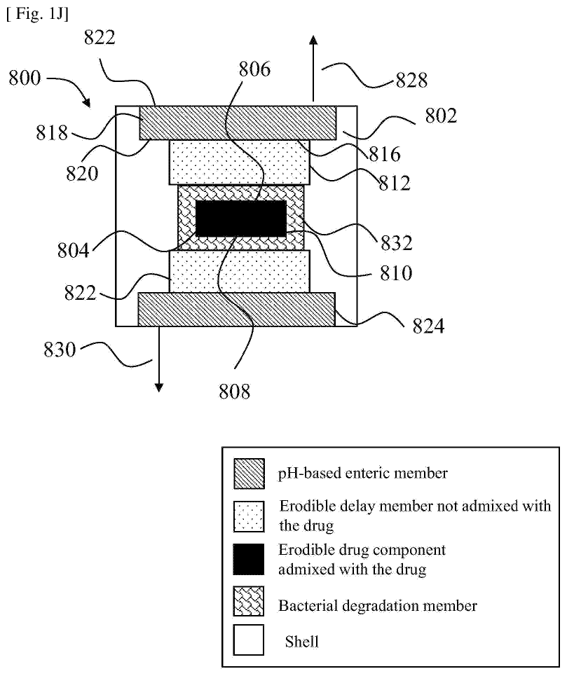

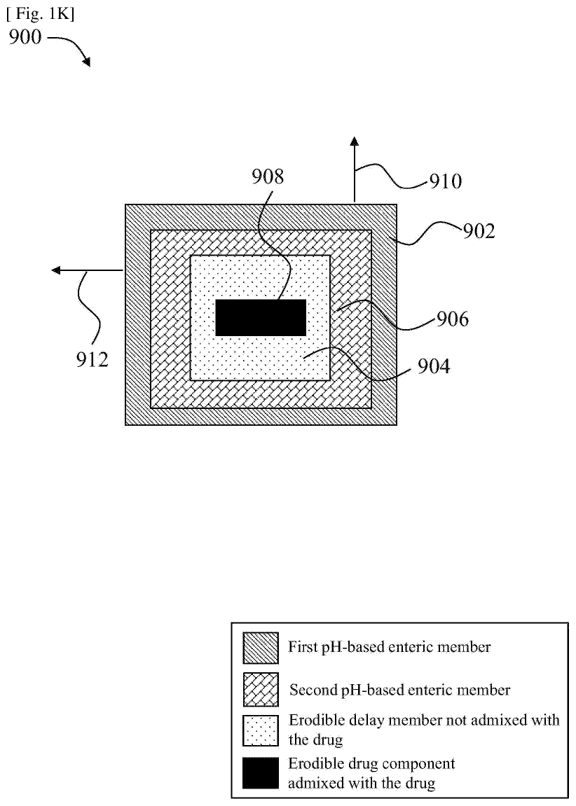

Example cross-sectional schematics of oral drug dosage forms configured to release a drug at a desired location in the colon are provided in FIGS. 1A-1H. As illustrated in FIG. 1A, in some embodiments, the oral drug dosage form 100 comprises a drug component 102 comprising an erodible material admixed with a drug; and a delay component not admixed with the drug, wherein the delay component is configured to prevent release of the drug from the drug component until the oral drug dosage form reaches the colon of the individual following administration, and wherein the delay component comprises a pH-based enteric member 104 comprising an erodible material configured to erode at or above a pre-determined pH value. Following administration, the exterior surfaces, constituting the pH-based enteric member 104, are exposed to gastrointestinal fluid in the individual. Once a pH threshold is met, e.g., as based on a pH in or near the colon, or a portion thereof, the material at the surfaces of the pH-based enteric member 104 erode from the oral drug dosage form, such as shown by the direction of the arrows 106, 108, 110, and 112. Based on the design of the pH-based enteric member 104, the drug component 102, or portions thereof (such as surfaces), can be controllably exposed to gastrointestinal fluid to control the release of the drug from the oral drug dosage form 100. For example, by having a thinner thickness (as assessed along the direction of erosion and compared to, e.g., the side thicknesses) of the pH-based enteric member 104 on top of and below the drug component 102, the erosion of the pH-based enteric member 104 will first expose a top surface 114 and bottom surface 116 of the drug component 102. Drug admixed with the drug component 102 can then be released from the oral drug dosage form 100, such as by erosion. The ability to design which aspect (s) of the drug component 102 are exposed to gastrointestinal fluid enable, at least in part, a controlled release of drug from the oral drug dosage form 100.

As illustrated in FIG. 1B, in some embodiments, the delay component further comprises an erodible delay member 204 not admixed with the drug. Following administration, the exterior surfaces of the oral drug dosage form 200, constituting the pH-based enteric member 202, are exposed to gastrointestinal fluid in the individual. Once a pH threshold is met, e.g., as based on a pH in or near the colon, or a portion thereof, the material at the surfaces of the pH-based enteric member 202 erode from the oral drug dosage form 200, such as shown by the direction of the arrows 208 and 210. Based on the design of the p H-based enteric member 202, the erodible delay member 204, or portions thereof (such as surfaces), can be controllably exposed to gastrointestinal fluid. Based on exposure to gastrointestinal fluid, the erodible delay member 204 will erode accordingly and then aspects of the drug component 206 will be exposed to gastrointestinal fluid thereby providing a release of the drug from the oral drug dosage form 100. Design of the delay component can expose certain aspects of the drug component 206 such as to enable, at least in part, a controlled release profile of the drug from the oral drug dosage form 200 is obtained.

As illustrated in FIGS. 1C and 1D, in certain aspects, the oral drug dosage forms provided herein, e.g., 300 and 400, comprise more than one component, e.g., drug components. Such dosage forms can be designed for sequential erosion (or at least partially sequential erosion), e.g., as shown for the drug components in FIG. 1C, or compartment-based erosion, e.g., as shown for the drug components in FIG. 1D. Specifically, as illustrated in FIG. 1C, following administration, the exterior surfaces of the oral drug dosage form 300, constituting the pH-based enteric member 302, are exposed to gastrointestinal fluid in the individual. Once a pH threshold is met, e.g., as based on a pH in or near the colon, or a portion thereof, the material at the surfaces of the pH-based enteric member 302 erode from the oral drug dosage form, such as shown by the direction of the arrows 310 and 312. Based on the design of the pH-based enteric member 302, the erodible delay member 304, or portions thereof (such as surfaces), can be controllably exposed to gastrointestinal fluid. Based on the exposure to gastrointestinal fluid, the erodible delay member 304 will erode accordingly and then aspects of the second drug component 306 will be exposed to gastrointestinal fluid thereby providing a release of a second drug from the oral drug dosage form 300. Based on the exposure to gastrointestinal fluid, the second drug component 306 will erode accordingly and then aspects of the drug component 308 will be exposed to gastrointestinal fluid thereby providing a release of the drug from the oral drug dosage form 300. Design of the delay component can expose certain aspects of the second drug component 306 such that a controlled release profile of the second drug from the oral drug dosage form 300 is obtained. Design of the delay component and/or second drug component 306 can expose certain aspects of the drug component 308 such that a controlled release profile of the drug from the oral drug dosage form 300 is obtained. The drug and the second drug can be the same or different. In some embodiments, an intermediate material is position between at least a portion of the second drug component 306 and the drug component 308, such as a second erodible delay member.

As illustrated in FIG. 1D, a compartment-based approach can be taken to take to designing an oral drug dosage form 400 taught herein. Following administration, the exterior surfaces of the oral drug dosage form 400, constituting the pH-based enteric member 402, are exposed to gastrointestinal fluid in the individual. Once a pH threshold is met, e.g., as based on a pH in or near the colon, or a portion thereof, the material at the surfaces of the pH-based enteric member 402 erode from the oral drug dosage form 400, such as shown by the direction of the arrows 410 and 412. Based on the design of the pH-based enteric member 402, the erodible delay member 404, or portions thereof (such as surfaces), can be controllably exposed to gastrointestinal fluid. Based on the exposure to gastrointestinal fluid, the erodible delay member 404 will erode accordingly and then aspects of the drug component 406 and the second drug component 408 will be exposed to gastrointestinal fluid thereby providing release of the drug and a second drug, respectively, from the oral drug dosage form 400. Design of the delay component can control drug release, such as achieving, e.g., separate drug release, simultaneous drug release, or partially overlapping drug release. In some embodiments, a second erodible delay member is associated with the second drug component 408 such that the second drug component 408 is not reliant upon the erodible delay member 404 for drug release. As described herein, oral drug dosage forms may contain many compartments containing drug and/or another aspect of the oral drug dosage form, e.g., a bacterial degradation moiety and/or an excipient.

As described herein, certain other components may be formulated in the oral drug dosage forms provided herein, e.g., a bacterial degradation moiety (such as admixed in a bacterial degradation member) and an excipient. For example, as illustrated in FIG. 1E, following administration, the exterior surfaces of the oral drug dosage form 500, constituting the pH-based enteric member 502, are exposed to gastrointestinal fluid in the individual. Once a pH threshold is met, e.g., as based on a pH in or near the colon, or a portion thereof, the material at the surfaces of the pH-based enteric member 502 erodes from the oral drug dosage form 500, such as shown by the direction of the arrows 510 and 512. Based on the design of the pH-based enteric member 502, the erodible delay member 504, or portions thereof (such as surfaces), can be controllably exposed to gastrointestinal fluid. Based on the exposure to gastrointestinal fluid, the erodible delay member 504 will erode accordingly and then aspects of bacterial degradation member ad mixed with a bacterial degradation moiety 506 will be exposed to gastrointestinal fluid. The bacterial degradation member 506 is then degraded to expose aspects of the drug component 508 thereby providing a release of the drug from the oral drug dosage form 500. Design of the delay component (in FIG. 1E, the delay component comprises the pH-based enteric member 502, the erodible delay member 504, and the bacterial degradation member 506) can expose certain aspects of the drug component 508 such that a controlled release profile of the drug from the oral drug dosage form 500 is obtained.

In certain aspects, the oral drug dosage forms provided herein comprise a shell. As illustrated in FIGS. 1F-1H, the oral drug dosage form 600 is configured to release a drug at a desired location of the colon, the oral drug dos age form comprising: a drug component comprising a first erodible material admixed with a drug; a shell not admixed with the drug, wherein the shell is associated with at least a portion of the drug component, such as via one or more sides of the drug component; and a delay component not admixed with the drug, the delay component comprising: a pH-based enteric member configured to erode at or above a pre-determined pH value; and an erodible delay member comprising an erodible material. As discussed in more detail below, and shown in FIG. 1F, the delay component comprises a pair of layers of the pH-based enteric member and a pair of layers of the erodible delay member that bookend the drug component, wherein the pH-based enteric member and the shell prevent erosion of the erodible delay member, and wherein the erodible delay member and the shell prevent erosion of the drug component.

A cross-sectional diagram of an oral drug dosage form 600 is provided in FIG. 1F. The oral drug dosage form 600 comprises a shell 602 comprising an insulating material that is impermeable to bodily fluids and a drug component 604 comprising a first erodible material admixed with a drug. As illustrated in FIG. 1F, the drug component 604 forms a layer comprising a top surface 606, a bottom surface 608, and one or more sides, e.g., 610, wherein the one or more sides of the layer of the drug component are associated with, such as in direct contact with, the shell. The use of directional terms, such as top, bottom, and sides, is intended to facilitate the description of the disclosure provided herein and is not to be construed as limiting the scope inventions provided herein. The delay component of the oral drug dosage form 600 is not admixed with the drug, and the delay component comprises: a pH-based enteric member configured to erode at or above a pre-determined pH value; and an erodible delay member comprising an erodible material, wherein the pH-based enteric member and the shell prevent erosion of the erodible delay member, and wherein the erodible delay member and the shell prevent erosion of the drug component. Specifically, in the oral drug dosage form 600 of FIG. 1F, the delay component comprises a pair of sets of layers of a first layer of the erodible delay member 612, a first layer of the pH-based enteric member 618, a second layer of the erodible delay member 622, and a second layer of the pH-based enteric member 624. The oral drug dosage form 600 is configured such that the top surface 606 of the layer of the drug component 604 is in direct contact with a bottom surface 614 of the first layer of the erodible delay member 612, and atop surface 616 of the first layer of the erodible delay member 612 is in direct contact with a bottom surface 620 of the first layer of the pH-based enteric member 618. The second set of layers of the delay components is arrange similarly, specifically, the bottom surface 608 of the layer of the drug component 604 is in direct contact with the second layer of the erodible delay member 622, and the second layer of the erodible delay member 622 is in direct contact with the second layer of the pH-based enteric member 624. As shown in FIG. 1F, the pH-based enteric member 618, 624 and the shell 602 completely surround the erodible delay member 612, 622 and the drug component 604.

The oral drug dosage form 600 of FIG. 1F is configured such that when administered to a human individual the oral drug dosage form will begin a journey through the human individual's gastrointestinal system, and once the oral drug dosage form 600 is subjected to a gastrointestinal fluid having a suitable pH value, such as a pH value of 5.5 or greater, a top surface 622 of the first layer of the pH-based enteric layer 618 and a bottom surface 626 of the second layer of the pH-based enteric layer 624 erode away from the oral drug dosage form 600 in the direction of the respective arrows 628, 630. As shown in FIG. 1G, at a certain time following administration to the human individual, the first layer and the second layer of the pH-based enteric member (618 and 624 as shown in FIG. 1F) have eroded away from the oral drug dosage form 600 and expose the top surface 616 the first layer of the erodible delay member 612 and a bottom surface 632 of the second layer of the erodible delay member 622 to a gastrointestinal fluid. The first layer and the second layer of the erodible delay member erode away from the oral drug dosage form 600 in the direction of the respective arrows 634, 636. As shown in FIG. 1H, at a certain time following administration to the human individual, the first layer and the second layer of the erodible delay member (612 and 622 as shown in FIGS. 1F and 1G) have eroded away from the oral drug dosage form 600 and expose the top surface 606 and the bottom surface 608 of the drug component 604, and the drug is released from the oral drug dosage form via erosion of the drug component 604, such in the direction of the arrows 638, 640. In some embodiments, the drug component breaks apart and at least a portion of the drug component may be released from the oral drug dosage form and further erode to release the drug in the human individual.

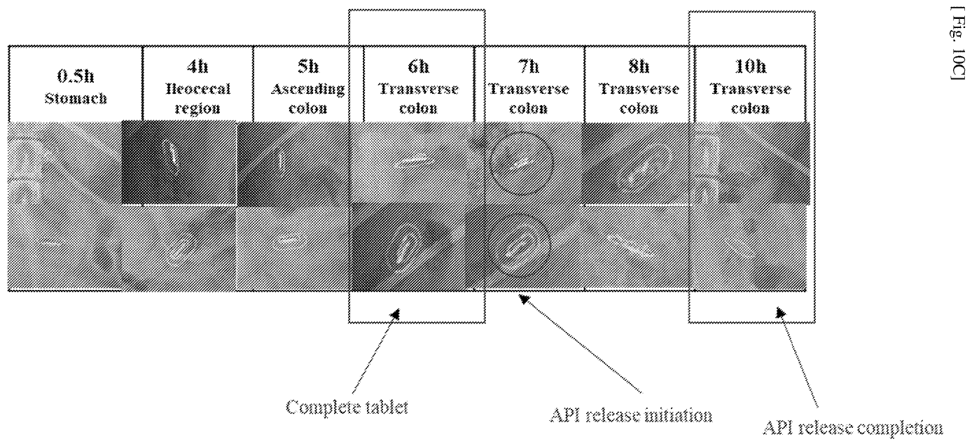

As described herein, certain other components may be formulated in the oral drug dosage forms provided herein, e.g., a bacterial degradation moiety (such as admixed in a bacterial degradation member) and an excipient. For example, as illustrated in FIG. 11. The oral drug dosage form 700 comprises a shell 702 comprising an insulating material that is impermeable to bodily fluids and a drug component 704 comprising a first erodible material admixed with a drug. As illustrated in FIG. 11, the drug component 704 forms a layer comprising a top surface 706, a bottom surface 708, and one or more sides, e.g., 710, wherein the one or more sides of the layer of the drug component are associated with, such as in direct contact with, the shell. The use of directional terms, such as top, bottom, and sides, is intended to facilitate the description of the disclosure provided herein and is not to be construed as limiting the scope inventions provided herein. The bacterial degradation members 732 and 734 are then degraded to expose aspects of the drug component 706 and 708 thereby providing a release of the drug from the oral drug dosage form 700. The delay component of the oral drug dosage form 700 is not admixed with the drug, and the delay component comprises: a pH-based enteric member configured to erode at or above a pre-determined pH value; and an erodible delay member comprising an erodible material, wherein the pH-based enteric member and the shell prevent erosion of the erodible delay member, and wherein the erodible delay member and the shell prevent erosion of the drug component. Specifically, in the oral drug dosage form 700 of FIG. 11, the delay component comprises a pair of sets of layers of a first layer of the erodible delay member 712, a first layer of the pH-based enteric member 718, a second layer of the erodible delay member 722, and a second layer of the pH-based enteric member 724. The oral drug dosage form 700 is configured such that the top surface 706 of the layer of the drug component 704 is in direct contact with a bottom surface 714 of the first layer of the erodible delay member 712, and a top surface 716 of the first layer of the erodible delay member 712 is in direct contact with a bottom surface 720 of the first layer of the pH-based enteric member 718. The second set of layers of the delay components is arrange similarly, specifically, the bottom surface 708 of the layer of the drug component 704 is in direct contact with the second layer of the erodible delay member 722, and the second layer of the erodible delay member 722 is in direct contact with the second layer of the pH-based enteric member 724. As shown in FIG. 11, the pH-based enteric member 718, 724 and the shell 702 completely surround the erodible delay member 712, 722, the bacterial degradation member 732, 734, and the drug component 704.

The present description provides additional formats of oral drug dosage forms. For example, as illustrated in FIG. 1J. The oral drug dosage form 800 comprises a shell 802 comprising an insulating material that is impermeable to bodily fluids and a drug component 804 comprising a first erodible material admixed with a drug. As illustrated in FIG. 1J, the drug component 804 forms a layer comprising a top surface 806, a bottom surface 808, and one or more sides, e.g., 810, wherein the one or more sides of the layer of the drug component are associated with, such as in direct contact with, the shell. The use of directional terms, such as top, bottom, and sides, is intended to facilitate the description of the disclosure provided herein and is not to be construed as limiting the scope inventions provided herein. The bacterial degradation member 832 is then degraded to expose aspects of the drug component 806 and 808 thereby providing a release of the drug from the oral drug dosage form 800. The delay component of the oral drug dosage form 800 is not admixed with the drug, and the delay component comprises: a pH-based enteric member configured to erode at or above a pre-determined pH value; and an erodible delay member comprising an erodible material, wherein the pH-based enteric member and the shell prevent erosion of the erodible delay member, and wherein the erodible delay member and the shell prevent erosion of the drug component. Specifically, in the oral drug dosage form 800 of FIG. 1J, the delay component comprises a pair of sets of layers of a first layer of the erodible delay member 812, a first layer of the pH-based enteric member 818, a second layer of the erodible delay member 822, and a second layer of the pH-based enteric member 824. The oral drug dosage form 800 is configured such that the top surface 806 of the layer of the drug component 804 is in direct contact with a bottom surface 814 of the first layer of the erodible delay member 812, and a top surface 816 of the first layer of the erodible delay member 812 is in direct contact with a bottom surface 820 of the first layer of the pH-based enteric member 818. The second set of layers of the delay components is arrange similarly, specifically, the bottom surface 808 of the layer of the drug component 804 is in direct contact with the second layer of the erodible delay member 822, and the second layer of the erodible delay member 822 is in direct contact with the second layer of the pH-based enteric member 824. As shown in FIG. 1J, the pH-based enteric member 818, 824 and the shell 802 completely surround the erodible delay member 812, 822, the bacterial degradation member 832, and the drug component 804. In some embodiments, the oral drug dosage form is configured such that the drug component releases from the oral drug dosage form at some point following administration, e.g., following erosion of another component of the drug dosage form such as the bacterial degradation member. In such embodiments, release of the drug from the drug component will factor in, e.g., the surface area of the drug component exposed to a gastrointestinal fluid.

In certain aspects, the oral drug dosage forms provided herein comprise a first pH-based enteric member and a second pH-based enteric member. As illustrated in FIG. 1K, following administration, the exterior surfaces of the oral drug dosage form 900, constituting the first pH-based enteric member 902, are exposed to gastrointestinal fluid in the individual. Once the first pH threshold is met, e.g., as based on at or above a pH value of about 5.5, or a portion thereof, the mate rial of the first pH-based enteric member 902 erodes from the surface thereof, such as shown by the direction of the arrows 910 and 912. Based on the design of the first pH-based enteric member 902, the gastric juice can be prevented from penetrating into the oral drug dosage form 900. And then once the second pH threshold is met, e.g., as based on a pH in or near the colon, or a portion thereof, the material at the surfaces of the second pH-based enteric member 906 erodes from the oral drug dosage form 900. Based on the design of the second pH-based enteric member 906, the drug being released in the small intestine can be prevented. Based on the design of the first pH-based enteric member 902 and the second pH-based enteric member 906, the erodible delay member 904, or portions thereof (such as surfaces), can be controllably exposed to gastrointestinal fluid. Based on the expo sure to gastrointestinal fluid, the erodible delay member 904 will erode accordingly and thereby providing a release of the drug from the oral drug dosage form 900. Design of the delay component (in FIG. 1K, the delay component comprises the first pH-based enteric member 902, the second pH-based enteric member 906, and the erodible delay member 904) can expose certain aspects of the drug component 908 such that a controlled release profile of the drug from the oral drug dos age form 900 is obtained.

In some embodiments, description of the oral drug dosage forms provided herein, and associated methods, is facilitated by describing the thickness of a component, such as a layer of an erodible delay member. In some embodiments, the thickness of a component, such as a layer of an erodible delay member, is measured from a surface, such as top surface, in a direction substantially parallel with the direction of erosion. For example, as shown in FIG. 1G, the thickness of a layer of an erodible delay member not admixed with the drug 612 is provided.

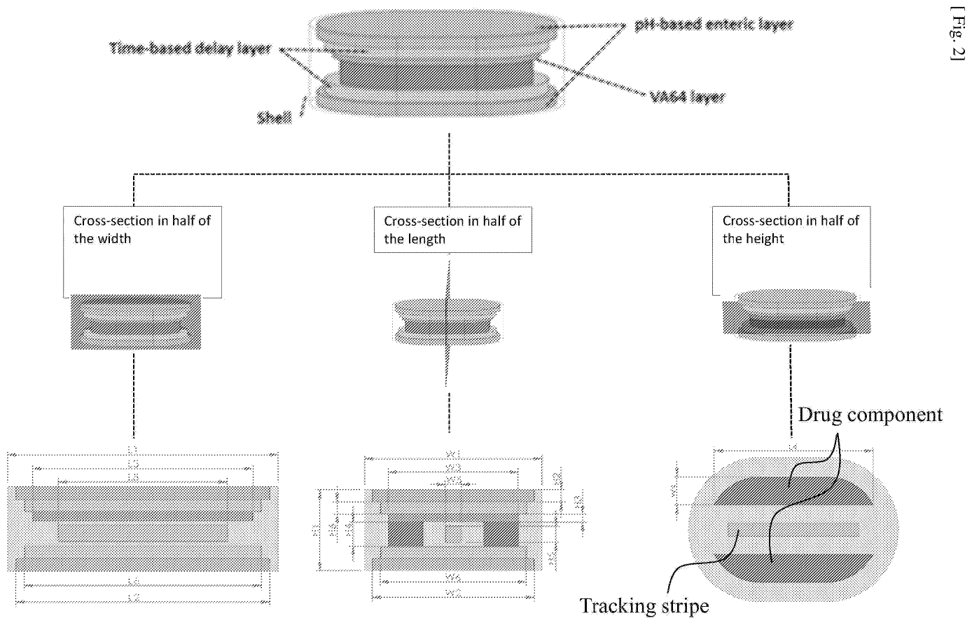

An example oral drug dosage form comprising a drug is provided in FIG. 2. As shown in FIG. 2, the oral drug dosage form comprises a shell not admixed with the drug, an internal drug component comprising a first erodible material admixed with a drug, and a delay component not admixed with the drug, wherein the delay component comprises two layers of a pH-based enteric member configured to erode at or above a pre-determined pH value and three layers of an erodible delay member comprising an erodible material, wherein each layer of the pH-based enteric member and the shell prevents erosion of a respective layer of the erodible delay member, and wherein the erodible delay member and the shell prevent erosion of the drug component. Following administration, erosion of the layers of the pH-based enteric member then exposes two layers of the erodible delay member to gastrointestinal fluid such that the two layers of the erodible delay member then begin to erode. Erosion of one of the layers of the erodible delay member exposes one surface of the drug component. Erosion of one of the layers of the erodible delay member exposes another layer of the erodible delay member (described as a VA64 layer, but not limited to such material) on one side of the drug component. Following erosion of the VA64 layer of the erodible delay member, another surface of the drug component is exposed. The expose surfaces of the drug component erode to release drug from the oral drug dosage form. As illustrated in FIG. 2, the oral drug dosage form may contain tracking features, such as tracking stripes and/or loops. Such tracking stripes are useful for development and optimization of an oral drug dosage form, e.g., imaging of an individual, such as using X-rays, following administration can be used to determine the location of erosion of aspects of the drug dosage form. Oral drug dosage forms without one or more of the tracking stripes is also encompassed by the disclosure provided herein. For example, following developments and optimization of the oral drug dosage form, the tracking stripes may no longer be needed. In such embodiments, the portion of the tracking stripe may be replaced by the material it is embedded in and/or additional adjustments can be made such as adjustment of one or more drug component features (e.g., surface area, thickness, drug mass fraction) to account for the tracking stripe no longer being present. The dimensions indicated in FIG. 2 are provided in Table 1A.

TABLE 1A
Dimensions of an oral drug dosage form pictured in FIG. 2.

| | Width (mm) | Length(mm) | Height (mm) |
|---|---|---|---|
| Shell | W1 = 8.8 | L1 = 12.8 | H1 = 4.0 |
| Layer of pH-based enteric | W2 = 8.0 | L2 = 12.0 | H2 = 0.6 |
| member (both layers have | | | |
| matching dimensions) | | | |
| Layer of erodible delay | W6 = 7.2 | L6 = 11.2 | H6 = 0.6 |
| member (both layers have | | | |
| matching dimensions | | | |
| VA64 layer of erodible delay | W3 = 6.4 | L3 = 10.8 | H3 = 0.4 |
| member | | | |
| Drug component | W4 = 1.7 | L4 = 9.7 | H4 = 1.2 |
| Tracking stripe | W5 = 0.8 | L5 = 8.0 | H5 = 0.8 |

In some embodiments, the shell comprises ethylcellulose (EC, e.g., EC-N10), dibutyl sebacate (DBS), and titanium dioxide (TiO₂). In some embodiments, the pH-based enteric member comprises vinyl acetate copolymer (VA64), Eudragit® L 100-55 (poly (methacrylic acid-co-ethyl acrylate; L100-55), and triethyl citrate (TEC). In some embodiments, the erodible delay member comprises hydroxypropyl cellulose (e.g., HPC JF), anhydrous dibasic calcium phosphate CaHPO₄), and TEC. In some embodiments, the erodible delay member, such as the VA64 layer, comprises VA64 and a polyethylene glycol (PEG, e.g., PEG4000). In some embodiments, the drug component comprises a drug, such as tofacitinib, e.g., tofacitinib citrate. In some embodiments, the drug component comprises a drug, such as tofacitinib, e.g., tofacitinib citrate, barium sulfate (BaSO₄), and a PEG, such as PEG20000. In some embodiments, the tracking strip comprises barium sulfate (BaSO₄) and a PEG, such as PEG20000.

In some embodiments, the oral drug dosage form of Table 1A comprises a weight by weight percentage (w/w %) and total weight of each component as listed in Table 1B.

TABLE 1B
Components of an oral drug dosage form pictured in FIG. 2.

| | Composition | Unit |
|---|---|---|
| Components | (% w/w) | (mg per tablet) |
| Eudragit ® L 100-55 | 4.96 | 20 |
| (poly(methacrylic acid-co-ethyl acrylate) | | |
| Vinyl acetate copolymer | 19.11 | 77 |
| Polyethylene Glycol 400 | 0.74 | 3 |
| Polyethylene Glycol 20000 | 2.47 | 9.97 |
| Triethyl Citrate | 7.34 | 29.6 |
| Hydroxypropyl Cellulose | 19.06 | 76.8 |
| (Klucel ™ JF Pharm) | | |
| Anhydrous Dibasic Calcium Phosphate | 2.38 | 9.6 |
| Tofacitinib citrate | 0.52 | 2.09 |
| Ethylcellulose | 33.75 | 136 |
| (Aqualon ™ EC-N10 Pharm) | | |
| Dibutyl Sebacate | 5.88 | 23.68 |
| Titanium Dioxide | 0.08 | 0.32 |
| Barium Sulfate | 3.71 | 14.94 |
| Total tablet weight | 100 | 403 |

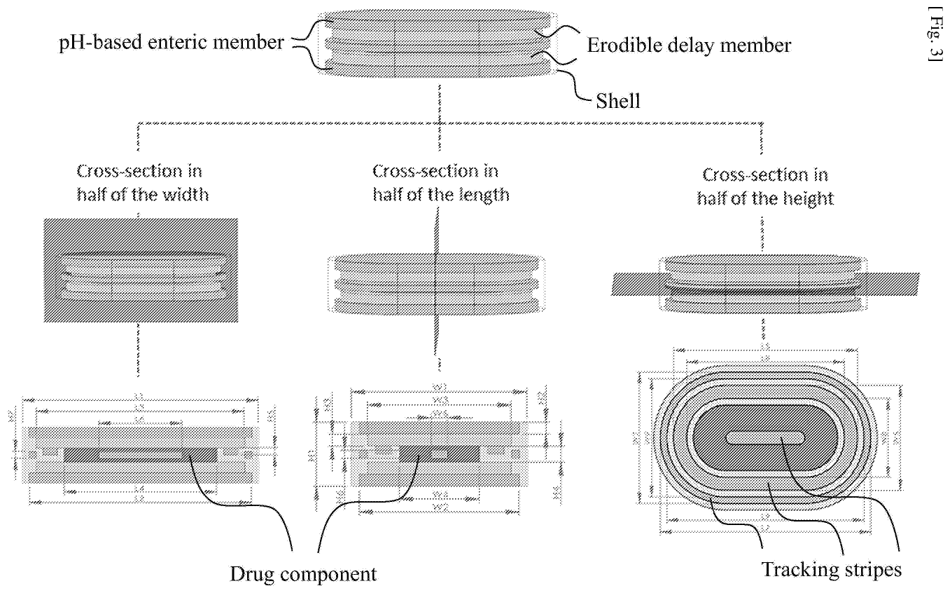

An example oral drug dosage form comprising a drug is provided in FIG. 3. As shown in FIG. 3, the oral drug dosage form comprises a shell not admixed with the drug, an internal drug component comprising a first erodible material admixed with a drug, and a delay component not admixed with the drug, wherein the delay component comprises two layers of a pH-based enteric member configured to erode at or above a pre-determined pH value and two layers of an erodible delay member comprising an erodible material, wherein each layer of the pH-based enteric member and the shell prevents erosion of a respective layer of the erodible delay member, and wherein the erodible delay member and the shell prevent erosion of the drug component. Following administration, erosion of the layers of the pH-based enteric member then exposes the layers of the erodible delay member to gastrointestinal fluid such that the layers of the erodible delay member then begin to erode. Erosion of the layers of the erodible delay member then exposes surfaces of the drug component such that erosion thereof releases drug from the oral drug dosage form. As illustrated in FIG. 3, the oral drug dosage form may contain tracking stripes. Such tracking stripes are useful for development and optimization of an oral drug dosage form, e.g., imaging of an individual, such as using X-rays, following administration can be used to determine the location of erosion of aspects of the drug dosage form. Oral drug dosage forms without one or more of the tracking stripes is also encompassed by the disclosure provided herein. For example, foll owing developments and optimization of the oral drug dosage form, the tracking stripes may no longer be needed. The dimensions indicated in FIG. 3 are provided in Table 2A.

TABLE 2A
Dimensions of an oral drug dosage form pictured in FIG. 3.

| | Width (mm) | Length(mm) | Height (mm) |
|---|---|---|---|
| Shell | W1 = 8.8 | L1 = 13.6 | H1 = 3.2 |
| Layer of pH-based enteric | W2 = 8.0 | L2 = 12.8 | H2 = 0.6 |
| member (both layers have | | | |
| matching dimensions) | | | |
| Layer of erodible delay | W3 = 7.2 | L3 = 12.0 | H3 = 0.6 |
| member (both layers have | | | |
| matching dimensions) | | | |
| Drug component | W4 = 4.0 | L4 = 8.8 | H4 = 0.8 |
| Tracking stripe | W5 = 6.4, | L5 = 11.2, | H5 = 0.4 |
| | W8 = 4.8 | L8 = 9.6 | |
| Tracking stripe | W6 = 0.8 | L6 = 5.6 | H6 = 0.4 |
| Tracking stripe | W7 = 8.0, | L7 = 12.8, | H7 = 0.4 |
| | W9 = 7.2 | L9 = 12.0 | |

In some embodiments, the shell comprises ethylcellulose (EC, e.g., EC-N10), dibutyl sebacate (DBS), and titanium dioxide (TiO₂). In some embodiments, the pH-based enteric member comprises vinyl acetate copolymer (VA64), Eudragit® L 100-55 (poly (methacrylic acid co-ethyl acrylate); L100-55), and triethyl citrate (TEC). In some embodiments, the erodible delay member comprises hydroxypropyl cellulose (e.g., HPC JF), calcium hydrophosphate (CaHPO₄), and TEC. In some embodiments, the drug component comprises a drug, such as tofacitinib, e.g., tofacitinib citrate. In some embodiments, the drug component comprises a drug, such as tofacitinib, e.g., tofacitinib citrate, VA64, and TEC In some embodiments, the tracking strip comprises barium sulfate (BaSO₄), VA64, and TEC.

In some embodiments, the oral drug dosage form of Table 2A comprises a weight by weight percentage (w/w %) and total weight of each component as listed in Table 2B.

TABLE 2B
Components of an oral drug dosage form pictured in FIG. 3.

| | Composition | Unit |
|---|---|---|
| Components | (% w/w) | (mg per tablet) |
| Eudragit ® L 100-55 | 5.92 | 21 |
| (poly(methacrylic acid co-ethyl acrylate) | | |
| Vinyl acetate copolymer | 24.34 | 86.35 |
| Triethyl Citrate | 10.63 | 37.72 |
| Hydroxypropyl Cellulose | 23.45 | 83.2 |
| (Klucel ™ JF Pharm) | | |
| Anhydrous Dibasic Calcium Phosphate | 2.93 | 10.4 |
| Tofacitinib citrate | 2.50 | 8.88 |
| Ethylcellulose | 23.11 | 82 |
| (Aqualon ™ EC-N10 Pharm) | | |
| Dibutyl Sebacate | 5.02 | 17.8 |
| Titanium Dioxide | 0.06 | 0.2 |
| Barium Sulfate | 2.03 | 7.2 |
| Total tablet weight | 100 | 354.75 |

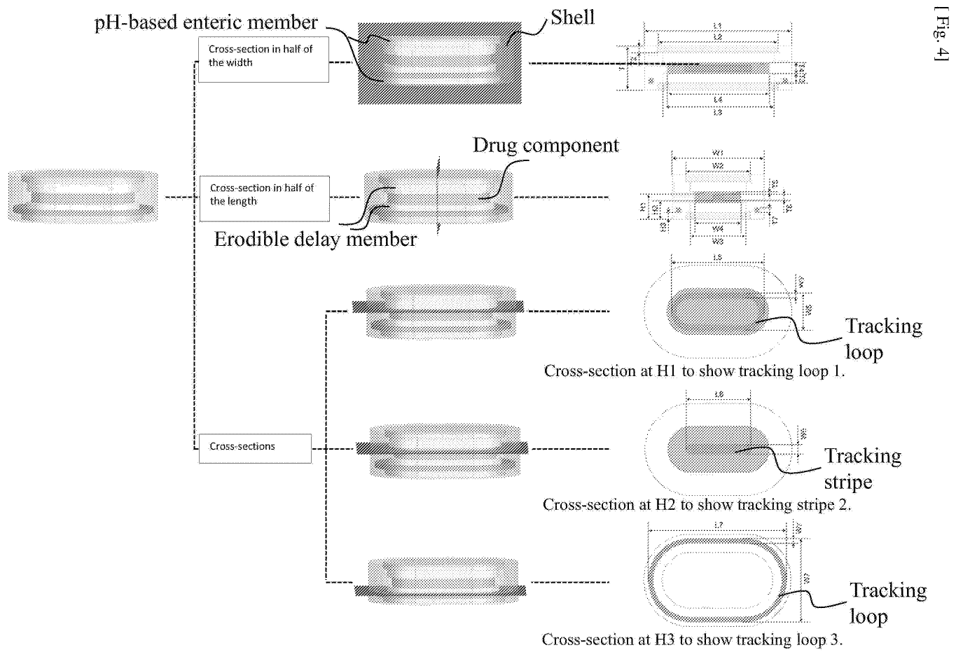

An example oral drug dosage form comprising a drug is provided in FIG. 4. As shown in FIG. 4, the oral drug dosage form comprises a shell not admixed with the drug, an internal drug component comprising a first erodible material admixed with a drug, and a delay component not admixed with the drug, wherein the delay component comprises two layers of a pH-based enteric member configured to erode at or above a pre-determined pH value and two layers of an erodible delay member comprising an erodible material, wherein each layer of the pH-based enteric member and the shell prevents erosion of a respective layer of the erodible delay member, and wherein the erodible delay member and the shell prevent erosion of the drug component. Following administration, erosion of the layers of the pH-based enteric member then exposes the layers of the erodible delay member to gastrointestinal fluid such that the layers of the erodible delay member then begin to erode. Erosion of the layers of the erodible delay member then exposes surfaces of the drug component such that erosion thereof releases drug from the oral drug dosage form. As illustrated in FIG. 4, the oral drug dosage form may contain tracking stripes. Such tracking stripes are useful for development and optimization of an oral drug dosage form, e.g., imaging of an individual, such as using X-rays, following administration can be used to determine the location of erosion of aspects of the drug dosage form. Oral drug dosage forms without one or more of the tracking stripes is also encompassed by the disclosure provided herein. For example, foll owing developments and optimization of the oral drug dosage form, the tracking stripes may no longer be needed. The dimensions indicated in FIG. 4 are provided in Table 3A.

TABLE 3A
Dimensions of an oral drug dosage form pictured in FIG. 4.

| | | | Thickness |
|---|---|---|---|
| | Width (mm) | Length(mm) | (mm) |
| Shell | W1 = 8.0 | L1 = 12.8 | T1 = 3.8 |
| Layer of pH-based enteric | W2 = 5.6 | L2 = 10.4 | T2 = 0.6 |
| member (both layers have | | | |
| matching dimensions) | | | |
| Layer of erodible delay | W3 = 4.8 | L3 = 9.6 | T3 = 0.8 |
| member (both layers have | | | |
| matching dimensions) | | | |
| Drug component | W4 = 4.0 | L4 = 8.8 | T4 = 1.0 |
| Tracking loop | W5 = 3.2, W5′ = 0.4 | L5 = 8.0 | T5 = 0.2 |
| Tracking stripe | W6 = 0.8 | L6 = 5.6 | T6 = 0.6 |
| Tracking loop | W7 = 7.2, W7′ = 0.4 | L7 = 12.0 | T7 = 0.4 |

In some embodiments, the shell comprises ethylcellulose (EC, e.g., EC-N10), dibutyl sebacate (DBS), and titanium dioxide (TiO₂). In some embodiments, the pH-based enteric member comprises vinyl acetate copolymer (VA64), Eudragit® L 100-55 (poly (methacrylic acid-co-ethyl acrylate); L100-55), triethyl citrate (TEC), and titanium dioxide (TiO₂). In some embodiments, the erodible delay member comprises hydroxypropyl cellulose (e.g., HPC JF), TEC, vitamin E polyethylene glycol-1000 succinate (TPGS), and TiO₂. In some embodiments, the drug component comprises a drug, such as tofacitinib, e.g., tofacitinib citrate. In some embodiments, the drug component comprises a drug, such as tofacitinib, e.g., tofacitinib citrate, VA64, and TEC. In some embodiments, the tracking strip or tracking loop comprises barium sulfate (BaSO₄), VA64, and TEC.

In some embodiments, the oral drug dosage form of Table 3A comprises a weight by weight percentage (w/w %) and total weight of each component as listed in Table 3B.

TABLE 3B
Components of an oral drug dosage form pictured in FIG. 4.

| | Composition | Unit |
|---|---|---|
| Components | (% w/w) | (mg per tablet) |
| Eudragit ® L 100-55 (poly(methacrylic | 3.47 | 12.8 |
| acid-co-ethyl acrylate) | | |
| Vitamin E polyethylene glycol-1000 | 1.00 | 3.70 |
| succinate(TPGS) | | |
| Vinyl acetate copolymer | 16.79 | 62.01 |
| Triethyl Citrate | 8.20 | 30.27 |
| Hydroxypropyl Cellulose | 15.97 | 58.97 |
| (Klucel ™ JF Pharm) | | |
| Tofacitinib citrate | 2.40 | 8.88 |
| Ethylcellulose | 41.08 | 151.73 |
| (Aqualon ™ EC-N10 Pharm) | | |
| Dibutyl Sebacate | 8.92 | 32.93 |
| Titanium Dioxide | 0.18 | 0.65 |
| Barium Sulfate | 2.01 | 7.42 |
| Total tablet weight | 100 | 369.36 |

An example oral drug dosage form comprising a drug is provided in FIG. 4. As shown in FIG. 4, the oral drug dosage form comprises a shell not admixed with the drug, an internal drug component comprising a first erodible material admixed with a drug, and a delay component not admixed with the drug, wherein the delay component comprises two layers of a pH-based enteric member configured to erode at or above a pre-determined pH value and two layers of an erodible delay member comprising an erodible material, wherein each layer of the pH-based enteric member and the shell prevents erosion of a respective layer of the erodible delay member, and wherein the erodible delay member and the shell prevent erosion of the drug component. Following administration, erosion of the layers of the pH-based enteric member then exposes the layers of the erodible delay member to gastrointestinal fluid such that the layers of the erodible delay member then begin to erode. Erosion of the layers of the erodible delay member then exposes surfaces of the drug component such that erosion thereof releases drug from the oral drug dosage form. As illustrated in FIG. 4, the oral drug dosage form may contain tracking stripes. Such tracking stripes are useful for development and optimization of an oral drug dosage form, e.g., imaging of an individual, such as using X-rays, following administration can be used to determine the location of erosion of aspects of the drug dosage form. Oral drug dosage forms without one or more of the tracking stripes is also encompassed by the disclosure provided herein. For example, foll owing developments and optimization of the oral drug dosage form, the tracking stripes may no longer be needed. The dimensions indicated in FIG. 4 are provided in Table 4A.

TABLE 4A
Dimensions of an oral drug dosage form pictured in FIG. 4.

| | Width | Length | Thickness |
|---|---|---|---|
| | (mm) | (mm) | (mm) |
| Shell | W1 = 8.0 | L1 = 12.8 | T1 = 3.83 |
| Layer of pH-based enteric | W2 = 5.6 | L2 = 10.4 | T2 = 0.6 |
| member (both layers have | | | |
| matching dimensions) | | | |
| Layer of erodible delay | W3 = 4.8 | L3 = 9.6 | T3 = 0.84 |
| member (both layers have | | | |
| matching dimensions | | | |
| Drug component | W4 = 4.0 | L4 = 8.8 | T4 = 0.95 |
| Tracking loop | W5 = 3.2, W5′ = 0.4 | L5 = 8.0 | T5 = 0.19 |
| Tracking stripe | W6 = 0.8 | L6 = 5.6 | T6 = 0.57 |
| Tracking loop | W7 = 7.2, W7′ = 0.4 | L7 = 12.0 | T7 = 0.42 |

In some embodiments, the shell comprises ethylcellulose (EC, e.g., EC-N10), dibutyl sebacate (DBS), and titanium dioxide (TiO₂). In some embodiments, the pH-based enteric member comprises vinyl acetate copolymer (VA64), Eudragit® L 100-55 (poly (methacrylic acid-co-ethyl acrylate); L100-55), triethyl citrate (TEC), and titanium dioxide (TiO₂). In some embodiments, the erodible delay member comprises hydroxypropyl cellulose (e.g., HPC JF), TEC, vitamin E polyethylene glycol-1000 succinate (TPGS), and TiO₂. In some embodiments, the drug component comprises a drug, such as tofacitinib, e.g., tofacitinib citrate, VA64, and TEC. In some embodiments, the drug component comprises a drug, such as tofacitinib, e.g., tofacitinib citrate. In some embodiments, the tracking strip or tracking loop comprises barium sulfate (BaSO₄), VA64, and TEC.

In some embodiments, the oral drug dosage form of Table 4A comprises a weight by weight percentage (w/w %) and total weight of each component as listed in Table 4B.

TABLE 4B
Components of an oral drug dosage form pictured in FIG. 4.

| | Composition | Unit |
|---|---|---|
| Components | (% w/w) | (mg per tablet) |
| Eudragit ® L 100-55 (poly(methacrylic | 3.42 | 12.8 |
| acid-co-etbyl acrylate) | | |
| Vitamin E polyethylene glycol-1000 | 1.04 | 3.88 |
| succinate (TPGS) | | |
| Vinyl acetate copolymer | 15.73 | 58.90 |
| Triethyl Citrate | 8.04 | 30.08 |
| Ilydroxypropyl Cellulose | 16.54 | 61.92 |
| (Klucel ™ JF Pharm) | | |
| Tofacitinib citrate | 2.37 | 8.88 |
| Ethylcellulose | 40.85 | 152.93 |
| (Aqualon ™ EC-N10 Pharm) | | |
| Dibutyl Sebacate | 8.87 | 33.20 |
| Titanium Dioxide | 0.18 | 0.66 |
| Barium Sulfate | 2.97 | 11.10 |
| Total tablet weight | 100 | 374.35 |

An example oral drug dosage form comprising a drug is described below based on the dimension descriptors provided in FIG. 11. The oral drug dosage form of FIG. 11 does not comprise a shell. As shown in FIG. 11, the oral drug dosage form comprises an internal drug component comprising a first erodible material admixed with a drug, and a delay component not admixed with the drug, wherein the delay component comprises a pH-based enteric member configured to erode at or above a pre-determined pH value and an erodible delay member comprising an erodible material. Following administration, erosion of the pH-based enteric member then exposes at least a portion of the erodible delay member to gastrointestinal fluid such that the erodible delay member (or portions thereof) then begin to erode. Erosion of the erodible delay member then exposes one or more surfaces of the drug component such that erosion thereof releases drug from the oral drug dosage form. As illustrated in FIG. 11, the oral drug dosage form may contain a tracking feature, such as a tracking stripe or loop (such terms may be used interchangeably). Such tracking stripes are useful for development and optimization of an oral drug dosage form, e.g., imaging of an individual, such as using X-rays, following administration can be used to determine the location of erosion of aspects of the drug dosage form. Oral drug dosage forms without one or more of the tracking stripes are also encompassed by the disclosure provided herein. For example, following development and optimization of the oral drug dosage form, the tracking stripes may no longer be needed. The dimensions indicated in FIG. 11 are provided in Table 5A. H1 is 2.38 mm and H2 is 1.92 mm.

TABLE 5A
Dimensions of an oral drug dosage form pictured in FIG. 11.

| | Width | Length | Thickness |
|---|---|---|---|
| | (mm) | (mm) | (mm) |
| pH-based enteric member | W1 = 7.6 | L1 = 15.0 | T1 = 4.3 |
| Erodible delay member | W2 = 6.8 | L2 = 14.2 | T2 = 4.0 |
| Drug component | W3 = 3.2 | L3 = 10.6 | T3 = 0.92 |
| Tracking loop | W4 = 3.2, W4′ = 0.4 | L4 = 10.6 | T4 = 0.23 |
| Tracking stripe | W5 = 0.8 | L5 = 8.2 | T5 = 0.46 |

In some embodiments, the pH-based enteric member comprises hypromellose acetate succinate AS-LG (HPMCAS LG), triethyl citrate (TEC), and titanium dioxide (TiO₂). In some embodiments, the erodible delay member comprises hydroxy propyl cellulose (e.g., HPC JF), TEC, vitamin E polyethylene glycol-1000 succinate (TPGS), and TiO₂. In some embodiments, the drug component comprises a drug, such as tofacitinib, e.g., tofacitinib citrate. In some embodiments, the drug component comprises a drug, such as tofacitinib, e.g., tofacitinib citrate, VA64, and TEC. In some embodiments, the drug component comprises a drug such as tofacitinib, e.g., tofacitinib citrate. In some embodiments, the tracking strip or tracking loop comprises barium sulfate (BaSO₄), VA64, and TEC.

In some embodiments, the oral drug dosage form of Table 5A comprises a weight by weight percentage (w/w %) and total weight of each component as listed in Table 5B.

TABLE 5B
Components of an oral drug dosage form pictured in FIG. 11.

| | Composition | Unit |
|---|---|---|
| Components | (% w/w) | (mg per tablet) |
| Hypromellose Acetate Succinate AS-LG | 17.13 | 87.78 |
| Vitamin E polyethylene glycol-1000 | 3.55 | 18.20 |
| succinate (TPGS) | | |
| Vinyl acetate copolymer | 3.54 | 18.12 |
| Triethyl Citrate | 15.93 | 81.61 |
| Hydroxypropyl Cellulose | 56.69 | 290.47 |
| (Klucel ™ JF Pharm) | | |
| Tofacitinib citrate | 1.73 | 8.88 |
| Titanium Dioxide | 0.19 | 0.95 |
| Barium Sulfate | 1.25 | 6.40 |
| Total tablet weight | 100 | 512.41 |

An example oral drug dosage form comprising a drug is described below based on the dimension descriptors provided in FIG. 11. The oral drug dosage form of FIG. 11 does not comprise a shell. As illustrated in FIG. 11, the oral drug dosage form may contain a tracking feature, such as a tracking stripe or loop (such terms may be used interchangeably). Such tracking stripes are useful for development and optimization of an oral drug dosage form, e.g., imaging of an individua 1, such as using X-rays, following administration can be used to determine the location of erosion of aspects of the drug dosage form. Oral drug dosage forms without one or more of the tracking stripes are also encompassed by the disclosure provided herein. For example, following development and optimization of the oral drug dosage form, the tracking stripes may no longer be needed. The dimensions indicated in FIG. 11 are provided in Table 6A. H1 is 1.59 mm and H2 is 1.13 mm.

TABLE 6A
Dimensions of an oral drug dosage form pictured in FIG. 11.

| | | Length | Thickness |
|---|---|---|---|
| | Width (mm) | (mm) | (mm) |
| pH-based enteric member | W1 = 7.6 | L1 = 15.0 | T1 = 2.72 |
| Erodible delay member | W2 = 6.8 | L2 = 14.2 | T2 = 2.42 |
| Drug component | W3 = 3.2 | L3 = 10.6 | T3 = 0.92 |
| Tracking loop | W4 = 3.2, W4′ = 0.4 | L4 = 10.6 | T4 = 0.23 |
| Tracking stripe | W5-0.8 | L5 = 8.2 | T5 = 0.46 |

In some embodiments, the pH-based enteric member comprises hypromellose acetate succinate AS-LG (HPMCAS LG), triethyl citrate (TEC), and titanium dioxide (TiO₂). In some embodiments, the erodible delay member comprises hydroxy propyl cellulose (e.g., HPC JF), TEC, vitamin E polyethylene glycol-1000 succinate (TPGS), and TiO₂. In some embodiments, the drug component comprises a drug, such as tofacitinib, e.g., tofacitinib citrate. In some embodiments, the drug component comprises a drug, such as tofacitinib, e.g., tofacitinib citrate, VA64, and TEC. In some embodiments, the drug component comprises a drug, such as tofacitinib, e.g., tofacitinib citrate. In some embodiments, the tracking strip or tracking loop comprises barium sulfate (BaSO₄), VA64, and TEC.

In some embodiments, the oral drug dosage form of Table 6A comprises a weight by weight percentage (w/w %) and total weight of each component as listed in Table 6B.

TABLE 6B
Components of an oral drug dosage form pictured in FIG. 11.

| | Composition | Unit |
|---|---|---|
| Components | (% w/w) | (mg per tablet) |
| Hypromellose Acetate Succinate AS-LG | 21.74 | 71.82 |
| Vitamin E polyethylene glycol-1000 | 3.06 | 10.10 |
| succinate (TPGS) | | |
| Vinyl acetate copolymer | 5.48 | 18.12 |
| Triethyl Citrate | 16.13 | 53.31 |
| Hydroxypropyl Cellulose | 48.79 | 161.20 |
| (Klucel ™ JF Pharm) | | |
| Tofacitinib citrate | 2.69 | 8.88 |
| Titanium Dioxide | 0.18 | 0.58 |
| Barium Sulfate | 1.94 | 6.40 |
| Total tablet weight | 100 | 330.41 |

An example oral drug dosage form comprising a drug is described below based on the dimension descriptors provided in FIG. 11. The oral drug dosage form of FIG. 11 does not comprise a shell. As illustrated in FIG. 11, the oral drug dosage form may contain a tracking feature, such as a tracking stripe or loop (such terms may be used interchangeably). Such tracking stripes are useful for development and optimization of an oral drug dosage form, e.g., imaging of an individua 1, such as using X-rays, following administration can be used to determine the location of erosion of aspects of the drug dosage form. Oral drug dosage forms without one or more of the tracking stripes are also encompassed by the disclosure provided herein. For example, following development and optimization of the oral drug dosage form, the tracking stripes may no longer be needed. The dimensions indicated in FIG. 11 are provided in Table 7A.

TABLE 7A
Dimensions of an oral drug dosage form pictured in FIG. 11.

| | Width | Length | Thickness |
|---|---|---|---|
| | (mm) | (mm) | (mm) |
| pH-based enteric member | W1 = 7.6 | L1 = 15.0 | T1 = 3.02 |
| Erodible delay member | W2 = 6.8 | L2 = 14.2 | T2 = 2.72 |
| Drug component | W3 = 3.2 | L3 = 10.6 | T3 = 0.92 |
| Tracking loop | W4 = 3.2, W4′ = 0.4 | L4 = 10.6 | T4 = 0.23 |
| Tracking stripe | W5 = 0.8 | L5 = 8.2 | T5 = 0.46 |

In some embodiments, the pH-based enteric member comprises hypromellose acetate succinate AS-LG (HPMCAS LG), triethyl citrate (TEC), and titanium dioxide (TiO₂). In some embodiments, the erodible delay member comprises hydroxy propyl cellulose (e.g., HPC JF), TEC, vitamin E polyethylene glycol-1000 succinate (TPGS), and TiO₂. In some embodiments, the drug component comprises a drug, such as tofacitinib, e.g., tofacitinib citrate. In some embodiments, the drug component comprises a drug, such as tofacitinib, e.g., tofacitinib citrate, VA64, and TEC. In some embodiments, the drug component comprises a drug, such as tofacitinib, e.g., tofacitinib citrate. In some embodiments, the tracking strip or tracking loop comprises barium sulfate (BaSO₄), VA64, and TEC.

In some embodiments, the oral drug dosage form of Table 7A comprises a weight by weight percentage (w/w %) and total weight of each component as listed in Table 7B.

TABLE 7B
Components of an oral drug dosage form pictured in FIG. 11.

| | Composition | Unit |
|---|---|---|
| Components | (% w/w) | (mg per tablet) |
| Hypromellose Acctate Succinate AS-LG | 20.57 | 75.56 |
| Vitamin E polyethylene glycol-1000 | 3.19 | 11.72 |
| succinate (TPGS) | | |
| Vinyl acetate copolymer | 4.93 | 18.12 |
| Triethyl Citrate | 16.05 | 58.97 |
| Hydroxypropyl Cellulose | 50.92 | 187.05 |
| (Klucel ™ JF Pharm) | | |
| Tofacitinib citrate | 2.42 | 8.88 |
| Titanium Dioxide | 0.18 | 0.65 |
| Barium Sulfate | 1.74 | 6.40 |
| Total tablet weight | 100 | 367.35 |

An example oral drug dosage form comprising a drug is described below based on the dimension descriptors provided in FIG. 11. The oral drug dosage form of FIG. 11 does not comprise a shell or tracking stripes. The dimensions indicated in FIG. 11 are provided in Table 8A.

TABLE 8A
Dimensions of an oral drug dosage form pictured in FIG. 11.

| | Width | Length | Thickness |
|---|---|---|---|
| | (mm) | (mm) | (mm) |
| pH-based enteric member | W1 = 7.6 | L1 = 17.0 | T1 = 5.15 |
| Erodible delay member | W2 = 6.8 | L2 = 16.2 | T2 = 4.75 |
| Drug component | W3 = 2.6 | L3 = 12 | T3 = 1.25 |

In some embodiments, the pH-based enteric member comprises hypromellose acetate succinate AS-LG (HPMCAS LG), acetyl tributyl citrate (ATBC), and titanium dioxide (TiO₂). In some embodiments, the erodible delay member comprises hydroxy propyl cellulose (e.g., HPC JF), TEC, vitamin E polyethylene glycol-1000 succinate (TPGS), and TiO₂. In some embodiments, the drug component comprises a drug, such as tofacitinib, e.g., tofacitinib citrate. In some embodiments, the drug component comprises a drug, such as tofacitinib, e.g., tofacitinib citrate, VA64, hydroxypropyl cellulose (HPC SSL), and triacetin.

In some embodiments, the oral drug dosage form of Table 8A comprises a weight by weight percentage (w/w %) and total weight of each component as listed in Table 8B.

TABLE 8B
Components of an oral drug dosage form pictured in FIG. 11.

| | Composition | Unit |
|---|---|---|
| Components | (% w/w) | (mg per tablet) |
| Hypromellose Acetate Succinate AS-LG | 18.84 | 135.12 |
| Acetyl tributyl citrate (ATBC) | 6.30 | 45.16 |
| Vitamin E polyethylene glycol-1000 | 3.43 | 24.64 |
| succinate (TPGS) | | |
| Vinyl acetate copolymer | 2.29 | 16.43 |
| Triethyl Citrate | 10.30 | 73.92 |
| Hydroxypropyl Cellulose | 54.82 | 393.28 |
| (Klucel ™ JF Pharm) | | |
| Tofacitinib citrate | 1.24 | 8.88 |
| Titanium Dioxide | 0.11 | 0.82 |
| Hydroxypropyl cellulose (HPC SSL) | 1.55 | 11.1 |
| Triacetin | 1.11 | 7.99 |
| Total tablet weight | 100 | 717.34 |

### B. Components and Configurations of Oral Drug Dosage Forms Described Herein

In certain aspects, provided herein is an oral drug dosage form comprising the following components: (1) a drug component comprising an erodible material admixed with a drug; and (2) a delay component not admixed with the drug, wherein the delay component comprises a pH-based enteric member configured to erode at or above a pre-determined pH value; and, optionally, any one or more additional features described herein, e.g., (i) an erodible delay member comprising an erodible material; (ii) a bacterial degradation member comprising a bacterial degradation moiety; or (iii) an excipient component comprising an excipient. In any of the above embodiments, the oral drug dosage form may comprise a shell not admixed with the drug. Components described herein may take the form of one or more layers. In some embodiments, the delay component of the oral drug dosage form comprises more than one set of layers of a pH-based enteric member and, optionally, an erodible delay member (e.g., a first set of a first layer of a pH-based enteric member and a first layer of an erodible delay member, and a second set of a second layer of a pH-based enteric member and a second layer of an erodible delay member). Furthermore, components of an oral drug dosage form described herein may be arranged such that the oral drug dosage form comprises one or more compartments, e.g., a first compartment comprising a drug component and a second compartment comprising a bacterial degradation member and/or an excipient.

In some embodiments, provided is an oral drug dosage form configured to release a drug at a desired location in the colon of an individual, the oral drug dosage form comprising: a drug component comprising an erodible material admixed with the drug; and a delay component not admixed with the drug, wherein the delay component is configured to prevent release of the drug from the drug component until the oral drug dosage form reaches the colon of the individual following administration, and wherein the delay component comprises: a pH-based enteric member comprising an erodible material configured to erode at or above a pre-determined pH value.

In some embodiments, the oral drug dosage form does not comprise an erodible delay member, e.g., the oral drug dosage form consists essentially of the drug component and the pH-based enteric member. In some embodiments, the erodible material of the pH-based enteric member comprises one or more of a hypromellose acetate succinate, hydroxypropyl methyl cellulose phthalate, cellulose acetate propionate (CAP), poly (methacrylic acid-co-ethyl acrylate), or polyvinyl acetate phthalate (PVAP). In some embodiments, the hypromellose acetate succinate is HPMCAS LG/LF, HPMCAS MG/MF, or HPMCAS HG/LF. In some embodiments, the hydroxypropyl methyl cellulose phthalate is HPMCP HP-50 or HPMCP HP-55. In some embodiments, the poly (methacrylic acid-co-ethyl acrylate) is Eudragit L100-55 or Eudragit L100.

In some embodiments, provided is an oral drug dosage form configured to release a drug at a desired location in the colon of an individual, the oral drug dosage form comprising: a drug component comprising an erodible material admixed with the drug; and a delay component not admixed with the drug, wherein the delay component is configured to prevent release of the drug from the drug component until the oral drug dosage form reaches the colon of the individual following administration, and wherein the delay component comprises: a pH-based enteric member comprising an erodible material configured to erode at or above a pre-determined pH value, and an erodible delay member, and wherein the oral drug dosage form is configured such that the pH-based enteric member erodes prior to the erodible delay member. In some embodiments, the oral drug dosage form is configured such that the pH-based enteric member erodes prior to the erodible delay member. In such embodiments, it is not a requirement that the pH-based enteric member completely erodes prior to the start of erosion of the erodible delay member (although in some embodiments that will occur). In some embodiments, the pH-based enteric member prevents eros ion, at least in part, of the erodible delay member (such as directly, e.g., the pH-based enteric member is in contact with the erodible delay member), and the erodible delay member prevents erosion, at least in part, of the drug component (such as directly, e.g., the erodible delay member is in contact with the drug component). In some embodiments, the oral drug dosage form comprises one or more inter mediate materials positioned between other components of an oral drug dosage form, such as an intermediate material not admixed with the drug.

In embodiments provided herein, the oral drug dosage form comprises a second drug. The second drug may be positioned in and/or admixed with any aspects of the oral drug dosage form. In some embodiments, the second drug is admixed in the drug component. In some embodiments, the oral drug dosage form further comprises a second drug component admixed with the second drug (which can be the same or different than the drug of the drug component). The second drug component admixed with the second drug component can be positioned in a diverse array of configurations based on the desired release of the second drug and, in some embodiments, the drug. For example, in some embodiments, the drug component is embedded in the second drug component, wherein the oral drug dosage form is configured such that the pH-based enteric member erodes prior to the erodible delay member, the erodible delay member erodes prior to the second drug component, the second drug component erodes prior to the second erodible delay member, and the second erodible delay member erodes prior to the drug component. In some embodiments, the second drug component is located in a compartment separate from the drug component, e.g., allowing for independent release of the second drug and the drug. In some embodiments, the oral drug dosage form comprises a second drug component and a second erodible delay components. In such embodiment, the oral drug dosage form is configured such that the pH-based enteric member erodes prior to the erodible delay member, the erodible delay member erodes prior to the second drug component, the second drug component erodes prior to the second erodible delay member, and the second erodible delay member erodes prior to the drug component. The embodiments above can be extrapolated to the inclusion of additional drugs and drug components, such as two or more drugs, three or more drugs, four or more drugs, and five or more drugs.

In embodiments provided herein, the oral drug dosage form comprises a bacterial degradation moiety. The bacterial degradation moiety may be positioned in and/or admixed with aspects of the oral drug dosage form, however, generally the bacterial degradation moiety will not be admixed with the pH-based enteric member. In some embodiments, the bacterial degradation moiety is admixed with the erodible material of the erodible delay member. In some embodiments, the bacterial degradation moiety is admixed with the drug component. For example, in some embodiments, the drug component provides a controlled release of a drug in the colon, wherein the drug component further comprises a bacterial degradation moiety. In some embodiments, the oral drug dosage form comprises abacterial degradation member comprising an erodible material admixed with the bacterial degradation moiety. The bacterial degradation member can be positioned in a diverse array of configurations. For example, in some embodiments, the delay component further comprises a bacterial degradation member comprising an erodible material admixed with the bacterial degradation moiety, wherein the oral drug dosage form is configured such that the pH-based enteric member erodes prior to the erodible delay member, the erodible delay member erodes prior to the bacterial degradation member, and the bacterial degradation member erodes prior to the drug component. In some embodiments, the pH-based enteric member prevents erosion, at least in part, of the erodible delay member, the erodible delay member prevents erosion, at least in part, of the bacterial degradation member, and the bacterial degradation member prevents erosion, at least in part, of the drug component. In some embodiments, the bacterial degradation moiety and/or bacterial degradation member are located in a compartment separate from the drug component.

In embodiments provided herein, the oral drug dosage form comprises an excipient. The excipient may be positioned in and/or admixed with any aspects of the oral drug dosage form. In some embodiments, the excipient is admixed with the erodible material of the erodible delay member. In some embodiments, the excipient is admixed with the drug component. In some embodiments, the oral drug dosage form comprises an excipient component comprising an erodible material admixed with an excipient. The excipient component can be positioned in a diverse array of configurations. For example, in some embodiments, the excipient component further comprises an erodible material admixed with an excipient, wherein the oral drug dosage form is configured such that the pH-based enteric member erodes prior to an erodible delay member, the erodible delay member erodes prior to the excipient component, and the excipient component erodes prior to the drug component. In some embodiments, the excipient and/or excipient component are located in a compartment separate from the drug component.

In some embodiments, the excipient is an absorption enhancer. In some embodiments, the oral drug dosage form is configured for local delivery, wherein the oral drug dosage form does not comprise an excipient that is an absorption enhancer. In some embodiments, the oral drug dosage form is configured for systemic delivery, wherein the oral drug dosage form comprises an excipient that is an absorption enhancer.

In embodiments provided herein, the oral drug dosage form comprises an adjuvant. The adjuvant may be positioned in and/or admixed with any aspects of the oral drug dosage form. In some embodiments, the adjuvant is admixed with the erodible material of the erodible delay member. In some embodiments, the adjuvant is admixed with the drug component. In some embodiments, the oral drug dosage form comprises an adjuvant component comprising an erodible material admixed with an adjuvant. The adjuvant component can be positioned in a diverse array of configurations. For example, in some embodiments, the excipient component further comprises an erodible material admixed with an adjuvant, wherein the oral drug dosage form is configured such that the pH-based enteric member erodes prior to an erodible delay member, the erodible delay member erodes prior to the adjuvant component, and the adjuvant component erodes prior to the drug component. In some embodiments, the adjuvant and/or adjuvant component are located in a compartment separate from the drug component. In some embodiments, the adjuvant is selected based on the drug being a biological drug. For example, in some embodiments, when the drug is a peptide biological drug, the adjuvant is a protease scavenger and/or a protease inhibitor. In some embodiments, when the drug is nucleic acid biological drug, the adjuvant is lipid nanoparticle (LNP). In some embodiments, the lipid nanoparticle is selected from the group consisting of ionizable lipids, helper lipids, cholesterol and pegylated lipids (PEG-lipids).

In some embodiments, the oral drug dosage form does not comprise a shell. In some embodiments, the oral drug dosage form comprises a shell not admixed with a drug of a drug component. In such embodiments, the oral drug dosage form is configured such that the shell, or a portion thereof, and the delay member prevent erosion of the drug component. In some embodiments, the direction of erosion of the delay component and/or the drug component are based on the configuration of the shell.

For any of the oral drug dosage forms described herein, one or more aspects of the oral drug dosage form may be configured in the form of a layer, such as a layer having a top surface, a bottom surface, and a thickness. Additionally geometrical configurations of aspects of the oral drug dosage forms are envisioned herein, such as a drug component comprising a spherical shape, or a portion thereof. In some embodiments, one or more aspects of the oral drug dosage form may be configured in the form of a compartment. In some embodiments, the oral drug dosage form comprises one or more compartments comprising one or more drug components. In some embodiments, the oral drug dosage form comprises one or more compartments comprising one or more drug components and one or more additional aspects, such as an excipient and/or adjuvant component.

The oral drug dosage forms described herein may be, for example, any size, shape, or weight that is suitable for oral administration. In some embodiments the oral drug dosage form is suitable for oral administration to an individual, wherein the size, shape, and/or weight of the oral drug dosage form is based on an attribute of the individual. In some embodiments, the attribute of an individual is one or more of height, weight, or age. In some embodiments, the individual is an infant. In some embodiments, the individual is a child. In some embodiments, the individual is an adolescent. In some embodiments, the individual is an adult.

In some embodiments, the largest dimension crossing an oral drug dos age form, e.g., largest diameter, is about 1 mm to about 25 mm, such as any of about 2 mm to about 10 mm, about 5 mm to about 12 mm, about 8 mm to about 15 mm, about 5 mm to about 10 mm, or about 7 mm to about 9 mm. In some embodiments, the largest dimension crossing an oral drug dosage form, e.g., largest diameter, is less than about 25 mm, such as less than about any of 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, or 1 mm. In some embodiments, the largest dimension crossing an oral drug dosage form, e.g., largest diameter, is greater than about 1 mm, such as greater than about any of 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm. In some embodiments, the largest dimension crossing an oral drug dosage form, e.g., largest diameter, is about any of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm.

In some embodiments, the oral drug dosage form has a thickness of ab out 1 mm to about 25 mm, such as any of about 2 mm to about 10 mm, about 5 mm to about 12 mm, about 8 mm to about 15 mm, about 5 mm to about 10 mm, or about 7 mm to about 9 mm. In some embodiments, the oral drug dosage form has a thickness of less than about 25 mm, such as less than about any of 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, or 1 mm. In some embodiments, the oral drug dosage form has a thickness of greater than about 1 mm, such as greater than about any of 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm. In some embodiments, the oral drug dosage form has a thickness of about any of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm.

In some embodiments, the shape of an oral drug dosage form comprises a cylinder, oval, bullet shape, arrow head shape, triangle, arced triangle, square, arced square, rectangle, arced rectangle, diamond, pentagon, hexagon, octagon, half moon, almond, or a combination thereof.

In some embodiments, the shape of an oral drug dosage form comprises a cylinder, oval, bullet shape, arrow head shape, triangle, arced triangle, square, arced square, rectangle, arced rectangle, diamond, pentagon, hexagon, octagon, half moon, almond, or a combination thereof, wherein the largest dimension crossing the oral drug dosage form, e.g., largest diameter, is about any of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm.

In some embodiments, the total weight of an oral drug dosage form is about 20 mg to about 1500 mg, such as about any of about 50 mg to about 150 mg, about 150 mg to about 250 mg, about 160 mg to about 170 mg, about 250 mg to about 350 mg, about 350 mg to about 450 mg, about 450 mg to about 550 mg, about 550 mg to about 650 mg, about 650 mg to about 750 mg, about 750 mg to about 850 mg, about 850 mg to about 950 mg, about 950 mg to about 1050 mg, about 1050 mg to about 1150 mg, about 1150 mg to about 1250 mg, about 1250 mg to about 1350 mg, or about 1350 mg to about 1450 mg. In some embodiments, the total weight of an oral drug dosage form is less than about 1500 mg, such as less than about any of 1450 mg, 1400 mg, 1350 mg, 1300 mg, 1250 mg, 1200 mg, 1150 mg, 1100 mg, 1050 mg, 1000 mg, 950 mg, 900 mg, 850 mg, 800 mg, 750 mg, 700 mg, 650 mg, 600 mg, 550 mg, 500 mg, 475 mg, 450 mg, 425 mg, 400 mg, 375 mg, 350 mg, 325 mg, 300 mg, 275 mg, 250 mg, 225 mg, 200 mg, 175 mg, 150 mg, 125 mg, 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, or 25 mg. In some embodiments, the total weight of an oral drug dosage form is greater than about 20 mg, such as greater than about any of 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, or 1450 mg. In some embodiments, the total weight of an oral drug dosage form is about any of 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 160 mg, 165 mg, 170 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, or 1450 mg.

As disclosed herein, the components of the oral drug dosage forms may be configured in numerous shapes and sizes. Unless otherwise specified, reference to certain shapes, sizes, and measurements are reflective of the oral drug dosage form prior to administration to a human individual, e.g., prior to erosion of any components thereof. Further discussion of components of the oral drug dosage forms provided herein is included in the sections below. The modular discussion of such components does not limit the scope of the invention and one of ordinary skill in the art will readily appreciate how certain features from the sections below can be combined to for the oral drug dosage forms taught herein.

### 1. Drug Components

The oral drug dosage forms disclosed herein comprise a drug component comprising an erodible material admixed with a drug. The drug components encompassed herein may be formed using numerous materials (including materials having a range of drug mass fractions) having varying shapes, sizes, and erosion rates. In some embodiments, the oral drug dosage form comprises a second drug component comprising a drug, wherein the drug and/or the erodible material of the drug component and the second drug component can be the same or different. In some embodiments, the oral drug dosage form comprises more than one compartment, and various configurations of drug components are envisioned here such that a compartment may contain one drug component, more than one drug component, or no drug components. Further description of the drug component is applicable to additional drug components, such as a second drug component.

In some embodiments, the drug component is a layer. In some embodiments, the drug component comprises a plurality of beads, such as a layer comprising a plurality of nanoparticles.

In some embodiments, the drug component is surrounded, such as completely surrounded, by a delay component (e.g., a pH-based enteric member and, optionally, an erodible delay member and/or a shell). In some embodiments, the drug component is configured to have surfaces, including one or more surfaces designed to be exposed to a bodily fluid (e.g., a gastrointestinal fluid) following administration of the oral drug dosage form to a human individual and following erosion/dissolution of a delay component. In some embodiments, the drug component is a layer comprising a top surface and a bottom surface. In some embodiments, the drug component comprises one or more sides, such as a side that is substantially perpendicular to a top surface or bottom surface of a drug component. In some embodiments, at least one of the one or more sides of the drug components is associated with (such as in direct contact with) an aspect of the delay component an d/or a shell of the oral drug dosage form. For example, in some embodiments, the oral drug dosage form is configured such that after erosion of a delay component comprising a pH-based enteric member and, optionally, an erodible delay member, the drug component maintains its position relative to the shell for a period of time as at least a portion of the drug component erodes in the presence of a bodily fluid.

In some embodiments, the drug component is a pre-determined shape and/or surface area, such as configured to provide a desired release profile of drug from an oral drug dosage form. For example, in some embodiments, the drug component releases a drug from an oral drug dosage form based on erosion of the drug component at any interface exposed to a bodily fluid (such as a top surface and/or a bottom surface). In some embodiments, the drug component has a top surface and a bottom surface, such as delimited by exposure to a bodily fluid during administration to an individual. In some embodiments, the top surface and the bottom surface of the drug component are exposed to a bodily fluid at the same time. In some embodiments, the top surface and/or the bottom surface of the drug component, or at least a portion thereof, is flat (such as within a surface tolerance threshold as measured between two parallel planes). In some embodiments, the top surface and/or the bottom surface of the drug component is not flat, e.g., comprises certain features that extend beyond a top surface plane or surface tolerance threshold. The surface (s) of the drug component that are exposed to a bodily fluid, such as a top surface and/or a bottom surface, as based on the surface exposed to a bodily fluid, can have any shape. In some embodiments, the surface (s) of the drug component that are exposed to a bodily fluid have the shape of a capsule, circle, oval, bullet shape, arrow head shape, triangle, arced triangle, square, arced square, rectangle, arced rectangle, diamond, pentagon, hexagon, octagon, half moon, almond, or a combination thereof.

In some embodiments, the surface (s) of a drug component initially exposed to a bodily fluid, such as a top surface and a bottom surface, have a surface area of about 10 mm² to about 400 mm², such as any of about 20 mm² to about 200 mm², about 20 mm² to about 100 mm², about 20 mm² to about 60 mm², about 30 mm² to about 50 mm². In some embodiments, the surface (s) of a drug component initially exposed to a bodily fluid, such as a top surface and a bottom surface, have a surface area of at least about 20 mm², such as at least about any of 22 mm², 24 mm², 26 mm², 28 mm², 30 mm², 32 mm², 33 mm², 34 mm², 36 mm², 38 mm², 40 mm², 42 mm², 44 mm², 46 mm², 48 mm², 50 mm², 52 mm², 54 mm², 56 mm², 58 mm², 60 mm², 65 mm², 70 mm², 80 mm², 85 mm², 90 mm², 95 mm², 100 mm², 110 mm², 120 mm², 130 mm², 140 mm², 150 mm², 160 mm², 170 mm², 180 mm², 190 mm², 200 mm², 225 mm², 250 mm², 275 mm², 300 mm², 325 mm², 350 mm², 375 mm², or 400 mm². In some embodiments, the surface (s) of a drug component initially exposed to a bodily fluid, such as a top surface and a bottom surface, have a surface area of less than about 400 mm², such as less than about any of 400 mm², 375 mm², 350 mm², 325 mm², 300 mm², 275 mm², 250 mm², 225 mm², 200 mm², 190 mm², 180 mm², 170 mm², 160 mm², 150 mm², 140 mm², 130 mm², 120 mm², 110 mm², 100 mm², 95 mm², 90 mm², 85 mm², 80 mm², 75 mm², 70 mm², 65 mm², 60 mm², 58 mm², 56 mm², 54 mm², 52 mm², 50 mm², 48 mm², 46 mm², 44 mm², 42 mm², 40 mm², 38 mm², 36 mm², 34 mm², 32 mm², 30 mm², 28 mm², 26 mm², 24 mm², 22 mm², or 20 mm². In some embodiments, the surface (s) of a drug component initially exposed to a bodily fluid, such as a top surface and a bottom surface, have a surface area of about any of 20 mm², 21 mm², 22 mm², 23 mm², 24 mm², 25 mm², 26 mm², 27 mm², 28 mm², 29 mm², 30 mm², 31 mm², 32 mm², 33 mm², 34 mm², 35 mm², 36 mm², 37 mm², 38 mm², 39 mm², 40 mm², 41 mm², 42 mm², 43 mm², 44 mm², 45 mm², 46 mm², 47 mm², 48 mm², 49 mm², 50 mm², 51 mm², 52 mm², 53 mm², 54 mm², 55 mm², 56 mm², 57 mm², 58 mm², 59 mm², 60 mm², 65 mm², 70 mm², 80 mm², 85 mm², 90 mm², 95 mm², 100 mm², 110 mm², 120 mm², 130 mm², 140 mm², 150 mm², 160 mm², 170 mm², 180 mm², 190 mm², 200 mm², 225 mm², 250 mm², 275 mm², 300 mm², 325 mm², 350 mm², 375 mm², or 400 mm².

In some embodiments, the surface (s) of the drug component exposed to a bodily fluid are substantially consistent throughout the thickness of the drug component (such as determined by the distance of the drug component substantially perpendicular to a top surface and/or a bottom surface), e.g., as the drug component erodes the surface (s) exposed to the bodily fluid have the same surface area. In some embodiments, the area of the surface (s) of the drug component exposed to a bodily fluid are different at two or more points, e.g., as the drug component erodes the surface (s) exposed to the bodily fluid change such as increases and/or decreases in surface area during erosion of the drug component. In some embodiments, the shape of the surface (s) of the drug component exposed to a bodily fluid are consistent throughout the thickness of the drug component, e.g., as the drug component erodes the surface (s) exposed to the bodily fluid are the same shape. In some embodiments, the shape of the surface (s) of the drug component exposed to a bodily fluid are different at two or more points. In some embodiments, the drug component comprises a bottom surface having substantially the same surface area as that of a top surface of the drug component. In some embodiments, the drug component comprises a bottom surface having a different surface area as that of a top surface of the drug component.

In some embodiments, the surface of a drug component initially exposed to a bodily fluid, such as a top surface or a bottom surface, has a largest crossing dimension of about 5 mm to about 20 mm, such as any of about 5 mm to about 15 mm, about 6 mm to about 13 mm, or about 7 to about 11 mm. In some embodiments, the surface of a drug component initially ex posed to a bodily fluid, such as a top surface or a bottom surface, has a largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the surface of a drug component initially exposed to a bodily fluid, such as a top surface or a bottom surface, has a largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the surface of a drug component initially exposed to a bodily fluid, such as a top surface or a bottom surface, has a largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

In some embodiments, the surface of a drug component initially exposed to a bodily fluid, such as a top surface or a bottom surface, has a crossing dimension perpendicular to the largest crossing dimension of about 1 mm to about 15 mm, such as any of about 2 mm to about 15 mm, about 2 mm to about 6 mm, or about 1 to about 5 mm. In some embodiments, the surface of a drug component initially exposed to a bodily fluid, such as a top surface or a bottom surface, has a crossing dimension perpendicular to the largest crossing dimension of at least about 1 mm, such as at least about any of 2 mm, 3 mm, 4 mm, 5 mm, 6 mm, 7 mm, 8 mm, 9 mm, 10 mm, 11 mm, 12, mm, 13 mm, 14 mm, or 15 mm. In some embodiments, the surface of a drug component initially exposed to a bodily fluid, such as a top surface or a bottom surface, has a crossing dimension perpendicular to the largest crossing dimension of less than about 15 mm, such as less than about any of 14 mm, 13 mm, 12 mm, 11 mm, 10 mm, 9 mm, 8 mm, 7 mm, 6 mm, 5 mm, 4 mm, 3 mm, 2 mm, or 1 mm. In some embodiments, the surface of a drug component initially exposed to a bodily fluid, such as a top surface or a bottom surface, has a crossing dimension perpendicular to the largest crossing dimension of about any of 1 mm, 2 mm, 3 mm, 4 mm, 5 mm, 6 mm, 7 mm, 8 mm, 9 mm, 10 mm, 11 mm, 12 mm, 13 mm, 14 mm, or 15 mm.

In some embodiments, the drug component, such as a drug component layer, has a thickness of about 0.1 mm to about 5 mm, such as any of about 0.2 mm to ab out 2 mm, about 0.5 mm to about 1.5 mm, or about 0.8 mm to about 1.4 mm. In some embodiments, the drug component, such as a drug component layer, has a thickness of at least about 0.1 mm, such as at least about any of 0.2 mm, 0.3 mm, 0.4 mm, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5 mm. In some embodiments, the drug component, such as a drug component layer, has a thickness of less than about 5 mm, such as less than about any of 4.8 mm, 4.6 mm, 4.4 mm, 4.2 mm, 4.0 mm, 3.8 mm, 3.6 mm, 3.4 mm, 3.2 mm, 3.0 mm, 2.8 mm, 2.6 mm, 2.4 mm, 2.2 mm, 2.0 mm, 1.9 mm, 1.8 mm, 1.7 mm, 1.6 mm, 1.5 mm, 1.4 mm, 1.3 mm, 1.2 mm, 1.1 mm, 1.0 mm, 0.9 mm, 0.8 mm, 0.7 mm, 0.6 mm, 0.5 mm, 0.4 mm, 0.3 mm, 0.2 mm, or 0.1 mm. In some embodiments, the drug component, such as a drug component layer, has a thickness of about any of 0.1 mm, 0.2 mm, 0.3 mm, 0.4 mm, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5 mm.

In some embodiments, the drug component, such as a drug component layer, comprises a top surface and a bottom surface, wherein the thickness, as measured between the top surface and the bottom surface, is substantially consistent, such as within a 20% range of an average thickness.

In some embodiments, the drug component has a drug mass fraction (m_{F}) of a drug of about 0.05 to about 0.6, such as any of about 0.05 to about 0.1, about 0.2 to about 0.5, about 0.3 to about 0.4, or about 0.3 to about 0.35. In some embodiments, the drug component has a drug mass fraction (m_{F}) of a drug of at least about 0.05, such as at least about any of 0.1, 0.15, 0.2, 0.25, 0.3, 0.35, 0.4, 0.45, 0.5, 0.55, or 0.6. In some embodiments, the drug component has a drug mass fraction (m_{F}) of a drug of less than about 0.6, such as less than about any of 0.55, 0.5, 0.45, 0.4, 0.35, 0.3, 0.25, 0.2, 0.15, 0.1, or 0.05. In some embodiments, the drug component has a drug mass fraction (m_{F}) of a drug of about any of 0.05, 0.1, 0.15, 0.2, 0.25, 0.3, 0.35, 0.4, 0.45, 0.5, 0.55, or 0.6.

In some embodiments, the drug component is configured for an immediate drug release profile, e.g., the erodible material admixed with a drug erodes (dissolves) when contacted by a bodily fluid (such as gastrointestinal fluids) to release the drug from the oral drug dosage form according to an immediate drug release profile. In some embodiments, the drug component completely erodes, such as from the point of being contacted by gastrointestinal fluids, in a period of about 1 minute to about 1 hour, such as any of about 5 minutes to about 30 minutes, about 5 minutes to about 20 minutes, or about 10 minutes to about 20 minutes. In some embodiments, the drug component completely erodes, such as from the point of being contacted by gastrointestinal fluids, in a period of less than about 1 hour, such as less than about any of 55 minutes, 50 minutes, 45 minutes, 40 minutes, 35 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes or 1 minute.

In some embodiments, the drug component is configured for a sustained drug release profile, e.g., the erodible material admixed with a drug erodes (dissolves) when contacted by a bodily fluid (such as gastrointestinal fluids) to release the drug from the oral drug dosage form according to a sustained drug release profile. In some embodiments, the drug component completely erodes, such as from the point of being contacted by gastrointestinal fluids, in a period of about 1 hour to about 7 hours, such as any of about 2 hours to about 4 hours, such as about 2 hours to about 3 hours. In some embodiments, the drug component completely erodes, such as from the point of being contacted by gastrointestinal fluids, over a period of at least about 1 hour, such as at least about any of 2 hours, 2.5 hours, 3 hours, 3.5 hours, 4 hours, 4.5 hours, 5 hours, 5.5 hours, 6 hours, 6.5 hours, or 7 hours.

In some embodiments, the amount of a drug in a drug component is about 0.5 mg to about 500 mg. In some embodiments, the amount of a drug in a drug component is about 1 mg or more, such as about any of 5 mg or more, 10 mg or more, 15 mg or more, 20 mg or more, 25 mg or more, 30 mg or more, 35 mg or more, 40 mg or more, 45 mg or more, 50 mg or more, 55 mg or more, 60 mg or more, 65 mg or more, 70 mg or more 0.75 mg or more, 80 mg or more, 85 mg or more, 90 mg or more, 95 mg or more, 100 mg or more, 110 mg or more, 120 mg or more, 130 mg or more, 140 mg or more, 150 mg or more, 160 mg or more, 170 mg or more, 180 mg or more, 190 mg or more, 200 mg or more, 210 mg or more, 220 mg or more, 230 mg or more, 240 mg or more, 250 mg or more, 260 mg or more, 270 mg or more, 280 mg or more, 290 mg or more, 300 mg or more, 310 mg or more, 320 mg or more, 330 mg or more, 340 mg or more, 350 mg or more, 360 mg or more, 370 mg or more, 380 mg or more, 390 mg or more, 400 mg or more, 410 mg or more, 420 mg or more, 430 mg or more, 440 mg or more, 450 mg or more, 460 mg or more, 470 mg or more, 480 mg or more, 490 mg or more, or 500 mg or more.

In some embodiments, the weight ratio of the drug component to the delay component is about 1:10 to about 10:1, such as any of about 1:9, 1:8, 1:7, 1:6, 1:5, 1:4, 1:3, 1:2, 1:1, 2:1, 3:1, 4:1, 5:1, 6:1, 7:1, 8:1, or 9:1.

In some embodiments, the oral drug dosage form comprises one or more drug components. For example, in some embodiments, the oral drug dosage form comprises a drug component. In some embodiments, the oral drug dosage form comprises two drug components. In some embodiments, the oral drug dosage form comprises three drug components. In some embodiments, the oral drug dosage form comprises four drug components. In some embodiments, the oral drug dosage form comprises five drug components. In embodiments comprising more than one drug component, the drug components can be the same or different as compared to one another, such as having the same or different shape, size, and/or constitution

In some embodiments, the drug component, such as a drug component layer, comprises a thermoplastic material, for example a thermoplastic polymer. In some embodiments, the drug component, such as a drug component layer, comprises a material comprising any one or more of a matrix material, a plasticizer, or another additive, e.g., a filler, a binder, a lubricant, a glidant, and a disintegrant.

In some embodiments, the drug component comprises one or more of copolyvidone, polyvinylpyrrolidone-co-vinyl-acetate, polyvinylpyrrolidone-polyvinyl acetate copolymer, polyvinylpyrrolidone, hydroxypropyl cellulose, hydroxylpropyl methylcellulose or Hypromellose, polyethylene oxide, polyethylene glycol, polyvinyl caprolactam-polyvinyl acetate-polyethylene glycol graft copolymer, polyethylene glycol-polyvinyl alcohol graft copolymer, Kollicoat IR-polyvinyl alcohol, polyvinyl alcohol, aminoalkyl methacrylate copolymer E, hydroxypropyl methylcellulose acetate succinate hypromellose acetate succinate, hydroxypropyl methylcellulose phthalate, maltitol, lactose monohydrate, xylitol, isomalt, sucrose, glucose, or dextran 70.

In some embodiments, the plasticizer is any one or more of triethyl citrate, vitamine polyethylene glycol succinate, acetylated triethyl citrate, tributyl citrate, tributyl o-acetylcitrate, polyoxyl 15 hydroxystearate, peg-40 hydrogenated castor oil, polyoxyl 35 castor oil, dibutyl sebacate, diethylphthalate, glycerine, methyl 4-hydroxybenzoate, castor oil, oleic acid, triacetin, polyalkylene glycol, stearic acid, palmitic acid, malic acid, polyethylene glycol, lauroyl PEG-32 glycerides, poloxamer 188, poloxamer 407, acetyltributyl citrate, D-sorbitol, propylene glycol, diethyl phthalate, citric acid, glyceryl behenate, D-mannitol, polysorbate, sorbitan monostearate, sorbitan monooleate, or polyoxyl 40 stearate.

In some embodiments, the other additive is any one or more of acacia, alginate, alginic acid, aluminum acetate, butylparaben, butylated hydroxytoluene, citric acid, calcium carbonate, candelilla wax, croscarmellose sodium, confectioner sugar, colloidal silicone dioxide, cellulose, plain or anhydrous calcium phosphate, carnuba wax, corn starch, carboxymethylcellulose calcium, calcium disodium ethylenediaminetetraacetic acid, calcium hydrogen phosphate dehydrate, cetylpyridine chloride, calcium phosphate dibasic, calcium phosphate tribasic, dibasic calcium phosphate, disodium hydrogen phosphate, dimethicone, erythrosine sodium, ethylenediaminetetraacetic acid, gelatin, glyceryl monooleate, iron oxide, ferric oxide, iron oxide yellow, iron oxide red, lactose (hydrous, anhydrous, monohydrate, or spray dried), microcrystalline cellulose, magnesium carbonate, magnesium oxide, methyl paraben, polysorbate 80, propylene paraben, potassium bicarbonate, potassium sorbate, potato starch, phosphoric acid, polyoxyethylene (40) stearate, sodium starch glycolate, starch pregelatinized, sodium crossmellose, sodium lauryl sulfate, starch, silicon dioxide, sodium benzoate, sucrose, sorbic acid, sodium carbonate, saccharin sodium, sodium alginate, silica gel, sorbitan monooleate, sodium chloride, sodium metabisulfite, sodium citrate dehydrate, sodium starch, sodium carboxy methyl cellulose, succinic acid, sodium propionate, titanium dioxide, or talc.

### 2. Drugs of Oral Drug Dosage Forms

In some embodiments, the drug in a drug component of an oral drug dosage form described herein can be any drug. In some embodiments, the drug is an anti-inflammatory agent, non-steroidal anti-inflammatory agent, steroid, immunosuppressant, antibiotic, biological agent, anti-neoplastic agent, analgesic, anaesthetic, anticonvulsant, antidiabetic agent, antihistamine, anti-infective, antineoplastic, antiparkinsonian agent, antirheumatic agent, appetite stimulant, appetite suppressant, blood modifier, bone metabolism modifier, cardiovascular agent, central nervous system depressant, central nervous system stimulant, decongestant, dopamine receptor agonist, electrolyte, gastrointestinal agent, immunomodulator, muscle relaxant, narcotic, parasympathomimetic, sympathomimetic, sedative, hypnotic, or vaccine. In some embodiments, the vaccine is a peptide-based vaccine or a nucleic acid-based vaccine, such as an mRNA-based vaccine. In some embodiments, the nucleic acid-based vaccine comprises RNA such as transfer RNA (tRNA), ribosomal RNA (rRNA) or messenger RNA (mRNA).

In some embodiments, the anti-inflammatory agent is a JAK inhibitor. In some embodiments, the drug is a JAK inhibitor. A JAK inhibitor is an agent that interferes with the JAK-STAT signaling pathway, such an inhibitor of one or more members associated with the JAK-STAT signaling pathway (e.g., a JAKinib). Members associated with the JAK-STAT signaling pathway, and inhibitors thereof, are known in the art. See, e.g., Rawlings et al., J Cell Sci, 117, 2004; and Schwartz et al., Nat Rev Drug Discov, 17, 2017. In some embodiments, the JAK inhibitor is an inhibitor of any one or more of Janus kinase 1 (JAK1), Janus kinase 2 (JAK2), Janus kinase 3 (JAK3), or tyrosine kinase 2 (TYK2). In some embodiments, the JAK inhibitor is an inhibitor of JAK1 and JAK 3. In some embodiments, the JAK inhibitor is an inhibitor of JAK1, JAK3, and JAK2. In some embodiments, the JAK inhibitor is an inhibitor of JAK1, JAK3, JAK2, and TYK2. In some embodiments, the JAK inhibitor is an inhibitor of JAK1 and JAK2. In some embodiments, the JAK inhibitor is an inhibitor of JAK1, JAK2, and TYK2. In some embodiments, the JAK inhibitor is an inhibitor of all JAKs (apan-JAK inhibitor). In some embodiments, the drug is a JAK inhibitor (including a JAK 1 inhibitor and/or JAK 2 inhibitor and/or JAK 3 inhibitor), sphingosine 1-phosphate (SIP) receptor antagonist, SIP receptor 1 regulator, S1P receptor 5 regulator, a glucocorticoid receptor (GCR) antagonist, tyrosine kinase (TYK) 2 inhibitor, cyclo-oxygenase (COX) inhibitor, dihydrofolate reductase (DHFR) inhibitor, or epithelial mesenchymal transition (EMT) inhibit or, or an agent that acts in any one or more of the preceding manners.

In some embodiments, the drug is a SIP receptor antagonist, such as amiselimod or a pharmaceutical salt thereof. In some embodiments, the drug is amiselimod hydrochloride.

In some embodiments, the drug is a JAK1 inhibitor and a TYK2 inhibit or, such as brepocitinib or a pharmaceutical salt thereof. In some embodiments, the drug is brepocitinib tosylate.

In some embodiments, the drug is a GCR antagonist, such as budesonide or a pharmaceutical salt thereof. In some embodiments, the drug is a budesonide.

In some embodiments, the drug is a TYK2 inhibitor, such as deucravacitinib or a pharmaceutical salt thereof. In some embodiments, the drug is deucravacitinib hydrochloride.

In some embodiments, the drug is a S1P receptor 1 regulator, such as etrasimod or a pharmaceutical salt thereof. In some embodiments, the drug is etrasimod arginine.

In some embodiments, the drug is a JAK1 inhibitor, such as filgotinib or a pharmaceutical salt thereof. In some embodiments, the drug is filgotinib maleate.

In some embodiments, the drug is a JAK inhibitor, such as ivarmacitinib or a pharmaceutical salt thereof. In some embodiments, the drug is ivarmacitinib sulfate.

In some embodiments, the drug is a JAK1 inhibitor, JAK2 inhibitor, JAK3 inhibitor, and TYK2 inhibitor (e.g., a pan-Janus kinase inhibitor), such as izencitinib or a pharmaceutical salt thereof. In some embodiments, the drug is izencitinib.

In some embodiments, the drug is a COX inhibitor and a DHFR inhibitor, such as mesalamine or a pharmaceutical salt thereof. In some embodiments, the drug is mesalamine.

In some embodiments, the drug is a S1P receptor 1 regulator and a S1P receptor 5 regulator, such as ozanimod or a pharmaceutical salt thereof. In some embodiments, the drug is ozanimod hydrochloride.

In some embodiments, the drug is an EMT inhibitor and JAK3 inhibitor, such as ritlecitinib or a pharmaceutical salt thereof. In some embodiments, the drug is ritlecitinib.

In some embodiments, the drug is a JAK1 inhibitor, JAK2 inhibitor, and JAK3 inhibitor, such as tofacitinib or a pharmaceutical salt thereof. In some embodiments, the drug is tofacitinib citrate.

In some embodiments, the drug is a JAK1 inhibitor, such as upadacitinib or a pharmaceutical salt thereof. In some embodiments, the drug is upadacitinib tartrate.

In some embodiments, the JAK inhibitor is selected from the group consisting of tofacitinib, abrocitinb, baricitinib, cerdulatinib, cucurbitacin I, decernotinib, fedratinib, filgotinib, gandotinib, itacitinib, lestaurtinib, momelotinib, oclacitinib, pacritinib, peficitinib, ruxolitinib, solcitinib, upadacitinib, BMS-986165, CHZ868, and SHR0302, or a pharmaceutically acceptable salt thereof. In some embodiments, the oral drug dosage form comprises a plurality of JAK inhibitors, wherein each JAK inhibitor is selected from the group consisting of tofacitinib, abrocitinb, baricitinib, cerdulatinib, cucurbitacin I, decernotinib, fedratinib, filgotinib, gandotinib, itacitinib, lestaurtinib, momelotinib, oclacitinib, pacritinib, peficitinib, ruxolitinib, solcitinib, upadacitinib, BMS-986165, CHZ868, and SHR0302, or a pharmaceutically acceptable salt thereof.

In some embodiments, the JAK inhibitor is tofacitinib or a pharmaceutically acceptable salt thereof. In some embodiments, the JAK inhibitor is tofacitinib citrate, such as tofacitinib mono-citrate. In some embodiments, the JAK inhibitor is tofacitinib tartrate, such as tofacitinib mono-tartrate. In some embodiments, the JAK inhibitor is tofacitinib malate, such as tofacitinib mono-malate. In some embodiments, the JAK inhibitor is tofacitinib oxalate such as tofacitinib mono-oxalate.

In some embodiments, the JAK inhibitor is a pharmaceutically acceptable salt in an amorphous form. In some embodiments, the JAK inhibitor is a pharmaceutically acceptable salt in a crystalline form.

In some embodiments, the anti-inflammatory agent is mesalazine, sulphasalazine, or balsalazide.

In some embodiments, the non-steroidal anti-inflammatory agent is ibuprofen or diclofenac.

In some embodiments, the steroid is prednisolone, budesonide, or fluticasone.

In some embodiments, the immunosuppressant is azathioprine, cyclosporin, or methotrexate.

In some embodiments, the biological agent is a peptide. In some embodiments, the peptide is a linear peptide, e.g., insulin. In some embodiments, the peptide is a circular peptide, e.g., octreotide. In some embodiments the biological agent is an antibody, or a fragment thereof, such as a monoclonal antibody. In some embodiments, the biological agent is a nucleic acid, e.g, DNA, RNA, analogs thereof, or combinations thereof. In some embodiments, the peptide is octreotide. In some embodiments, the antibody is infliximab, adalimumab, certolizumab pegol, golimumab, or ustekinumab.

In some embodiments, the antineoplastic agent is fluorouracil, methotrexate, dactinomycin, bleomycin, etoposide, taxol, vincristine, doxorubicin, cisplatin, daunorubicin, Etoposide, raltitrexed, or oxaliplatin, or a combination thereof.

In some embodiments, the amount of a drug in an oral drug dosage form is about 0.5 mg to about 500 mg. In some embodiments, the amount of a drug in a drug component is about 1 mg or more, such as about any of 5 mg or more, 10 mg or more, 15 mg or more, 20 mg or more, 25 mg or more, 30 mg or more, 35 mg or more, 40 mg or more, 45 mg or more, 50 mg or more, 55 mg or more, 60 mg or more, 65 mg or more, 70 mg or more, 75 mg or more, 80 mg or more, 85 mg or more, 90 mg or more, 95 mg or more, 100 mg or more, 110 mg or more, 120 mg or more, 130 mg or more, 140 mg or more, 150 mg or more, 160 mg or more, 170 mg or more, 180 mg or more, 190 mg or more, 200 mg or more, 210 mg or more, 220 mg or more, 230 mg or more, 240 mg or more, 250 mg or more, 260 mg or more, 270 mg or more, 280 mg or more, 290 mg or more, 300 mg or more, 310 mg or more, 320 mg or more, 330 mg or more, 340 mg or more, 350 mg or more, 360 mg or more, 370 mg or more, 380 mg or more, 390 mg or more, 400 mg or more, 410 mg or more, 420 mg or more, 430 mg or more, 440 mg or more, 450 mg or more, 460 mg or more, 470 mg or more, 480 mg or more, 490 mg or more, or 500 mg or more. In some embodiments, the amount of a drug in a drug component is about 500 mg or less, such as about any of 490 mg or less, 480 mg or less, 470 mg or less, 460 mg or less, 450 mg or less, 440 mg or less, 430 mg or less, 420 mg or less, 410 mg or less, 400 mg or less, 390 mg or less, 380 mg or less, 370 mg or less, 360 mg or less, 350 mg or less, 340 mg or less, 330 mg or less, 320 mg or less, 310 mg or less, 300 mg or less, 290 mg or less, 280 mg or less, 270 mg or less, 260 mg or less, 250 mg or less, 240 mg or less, 230 mg or less, 220 mg or less, 210 mg or less, 200 mg or less, 190 mg or less, 180 mg or less, 170 mg or less, 160 mg or less, 150 mg or less, 140 mg or less, 130 mg or less, 120 mg or less, 110 mg or less, 100 mg or less, 90 mg or less, 80 mg or less, 70 mg or less, 60 mg or less, 50 mg or less, 40 mg or less, 30 mg or less, 20 mg or less, 10 mg or less, 5 mg or less, or 1 mg or less. In some embodiments, the amount of a drug in a drug component is about any of 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, or 500 mg.

### 3. Delay Components

The oral drug dosage forms described herein comprise a delay component configured to control, such as prevent and/or inhibit, the release of a drug from the oral drug dosage form so as to deliver the drug to desired location of the colon. As described herein, the delay component is not admixed with the drug of the drug component. In some embodiments, the delay components do not contain a drug, such as an active pharmaceutical ingredient. In some embodiments, the delay component comprises another drug. Moreover, aspects of the delay component may be admixed with another agent, such as a bacterial degradation moiety or an excipient.

In some embodiments, the delay component surrounds the drug component. In some embodiments, the delay component completely surrounds the drug component.

In some embodiments, the delay component (such as via aspects thereof) at least partially surround, e.g., sandwich, a drug component. For example, in some embodiments, the drug component is sandwiched between two erodible delay layers, which in turn are sandwiched between two pH-based enteric layers. In some embodiments, the delay component is formed from any number of layers of pH-based enteric layers and erodible delay layers. In some embodiments, when a drug component is at least partially surrounded, e.g., sandwiched, by layers of the delay component, the layers may form symmetric or asymmetric structures. For example, in some embodiments, the drug component is sandwiched between two erodible delay layers, which in turn are sandwiched between two pH-based enteric lay ers. In some embodiments, the drug component is sandwich between two erodible delay layers, wherein an additional erodible delay layer is added to a surf ace distal to the drug component of one of the two erodible delay layers, and wherein the drug component and the erodible delay layers are sandwiched between two pH-based enteric layers.

i. pH-Based Enteric Members

The delay components described herein comprise a pH-based enteric member, such as a pH-based enteric member layer. In some embodiments, the pH-based enteric member comprises more than one layer of a pH-based enteric material, such as two layers of a pH-based enteric material.

In some embodiments, the oral drug dosage form comprises one or more pH-based enteric members, wherein the one or more pH-based enteric members may be the same or different as compared to one another, such as having the same or different shape, size, and/or constitution. For example, in some embodiments, the oral drug dosage form comprises a first pH-based enteric member and a second pH-based enteric member. In some embodiments, the first pH-based enteric member and the second pH-based enteric member have a different pH wherein the respective materials start to erode. In some embodiments, the oral drug dosage form is configured such that a first pH-based enteric member erodes prior to a second pH-based enteric member being exposed to a bodily fluid following administration to an individual.

The pH-based enteric member is configured to erode at or above a desired pH value. As appreciated in the field, different regions of the human gastrointestinal tract have different pH environments. Use of such pH-based enteric members helps to control erosion of this component of the oral drug dosage forms described herein until a desired location of the gastrointestinal tract. In some embodiments, the pH-based enteric member erodes at a pH value of between about 5.5 to about 8, such as between any of about 5.5 to about 7.5, and about 6 to about 7. In some embodiments, the pH-based enteric member erodes at a pH value of, or above, about 5.5, such as about any of 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.

The pH-based enteric member may be formed using numerous materials having varying shapes and sizes. In some embodiments, the pH-based enteric member is a layer of a pH-based enteric material. In some embodiments, the pH-based enteric members are configured to have surfaces, such as a surface exposed to a bodily fluid during administration of the oral drug dosage form to a human individual, having a pre-determined shape and surface area. For example, in some embodiments, the pH-based enteric member has a top surface and a bottom surface, wherein the top surface is exposed to a bodily fluid prior to the bottom surface. In some embodiments, the pH-based enteric member is a layer having a top surface and a bottom surface. In some embodiments, the top surface of the pH-based enteric member is not flat, e.g., comprises certain features that extend beyond a top surface plane or surface tolerance threshold (as measured between two parallel planes), such as to reduce adherence of the pH-based enteric member, or a portion thereof, to an internal body part of the human individual. In some embodiments, the top surface of the pH-based enteric member, or at least a portion thereof, is flat or with in a surface tolerance threshold.

In some embodiments, the pH-based enteric member surrounds the drug component. In such embodiments, it is possible to not have or have additional material between the pH-based enteric member and the drug component, e.g., the presence of an erodible delay member.

The surfaces of a pH-based enteric member, such as a pH-based enteric member layer, as based on the surface exposed to a bodily fluid upon administration, can have any shape. In some embodiments, the surface has the shape of a capsule, circle, oval, bullet shape, arrow head shape, triangle, arced triangle, square, arced square rectangle, arced rectangle, diamond, pentagon, hexagon, octagon, half moon, almond, or a combination thereof.

In some embodiments, the surface of a pH-based enteric member, such as a pH-based enteric member layer, has a surface area of about 10 mm² to about 400 mm² such as any of about 20 mm² to about 200 mm², about 20 mm² to about 100 mm² about 20 mm² to about 60 mm², about 30 mm² to about 50 mm². In some embodiments, the surface of a pH-based enteric member has a surface area of at least about 20 mm², such as at least about any of 22 mm², 24 mm², 26 mm², 28 mm², 30 mm², 32 mm², 33 mm², 34 mm², 36 mm², 38 mm², 40 mm², 42 mm², 44 mm², 46 mm², 48 mm², 50 mm², 52 mm², 54 mm², 56 mm², 58 mm², 60 mm², 65 mm², 70 mm², 80 mm², 85 mm², 90 mm², 95 mm², 100 mm², 110 mm², 120 mm² 130 mm², 140 mm², 150 mm², 160 mm², 170 mm², 180 mm², 190 mm², 200 mm², 225 mm², 250 mm², 275 mm², 300 mm², 325 mm², 350 mm², 375 mm², or 400 mm². In some embodiments, the surface of a pH-based enteric member has a surface area of less than about 400 mm², such as less than about any of 400 mm², 375 mm², 350 mm², 325 mm², 300 mm², 275 mm², 250 mm², 225 mm², 200 mm², 190 mm², 180 mm², 170 mm², 160 mm², 150 mm², 140 mm², 130 mm², 120 mm², 110 mm², 100 mm², 95 mm², 90 mm², 85 mm², 80 mm², 75 mm², 70 mm², 65 mm², 60 mm², 58 mm², 56 mm², 54 mm², 52 mm², 50 mm², 48 mm², 46 mm², 44 mm², 42 mm², 40 mm², 38 mm², 36 mm², 34 mm², 32 mm², 30 mm², 28 mm², 26 mm², 24 mm², 22 mm², or 20 mm². In some embodiments, the surface of a pH-based enteric member has a surface area of about any of 20 mm², 21 mm², 22 mm², 23 mm², 24 mm², 25 mm², 26 mm², 27 mm², 28 mm², 29 mm², 30 mm², 31 mm², 32 mm², 33 mm², 34 mm², 35 mm², 36 mm², 37 mm², 38 mm², 39 mm², 40 mm², 41 mm², 42 mm², 43 mm², 44 mm², 45 mm², 46 mm², 47 mm², 48 mm², 49 mm², 50 mm², 51 mm², 52 mm², 53 mm², 54 mm², 55 mm², 56 mm², 57 mm², 58 mm², 59 mm², 60 mm², 65 mm², 70 mm², 80 mm², 85 mm², 90 mm², 95 mm², 100 mm², 110 mm², 120 mm², 130 mm², 140 mm², 150 mm², 160 mm², 170 mm², 180 mm², 190 mm², 200 mm², 225 mm², 250 mm², 275 mm², 300 mm², 325 mm², 350 mm², 375 mm², or 400 mm².

In some embodiments, the surface area of a surface of a pH-based enteric member exposed to a bodily fluid is consistent throughout the thickness of the pH-based enteric member, e.g., as a pH-based enteric member erodes the surface exposed to the bodily fluid has the same surface area. In some embodiments, the surface area of a surface of a pH-based enteric member exposed to a bodily fluid is different at two or more points, e.g., as a pH-based enteric member erodes the surface exposed to the bodily fluid changes such as increases and/or decreases in surface area during erosion of a pH-based enteric member. In some embodiments, the shape of the surface of a pH-based enteric member exposed to a bodily fluid is consistent throughout the thickness of the pH-based enteric member. In some embodiments, the shape of a surface of a pH-based enteric member exposed to a bodily fluid is different at two or more points.

In some embodiments, the surface of a pH-based enteric member, such as a pH-based enteric member layer, has a largest crossing dimension of about 5 mm to about 2 0 mm, such as any of about 5 mm to about 15 mm, about 6 mm to about 13 mm, or about 7 to about 11 mm. In some embodiments, the surface of a pH-based enteric member has a largest crossing dimension of at least ab out 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the surface of a pH-based enteric member has a largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the surface of a pH-based enteric member has a largest crossing dimension of about any of 5 mm, 6 mm, 7 mm, 8 mm, 9 mm, 10 mm, 11 mm, 12 mm, 13 mm, 14 mm, 1 5 mm, 16 mm, 17 mm, 18 mm, 19 mm, or 20 mm.

In some embodiments, the surface of a pH-based enteric member, such as a pH-based enteric member layer, has a crossing dimension perpendicular to the largest crossing dimension of about 1 mm to about 15 mm, such as any of about 2 mm to about 10 mm, about 2 mm to about 6 mm, or about 1 to about 5 mm. In some embodiments, the surface of a pH-based enteric member has a crossing dimension perpendicular to the largest crossing dimension of at least about 1 mm, such as at least about any of 2 mm, 3 mm, 4 mm, 5 mm, 6 mm, 7 mm, 8 mm, 9 mm, 10 mm, 11 mm, 12 mm, 13 mm, 14 mm, or 15 mm. In some embodiments, the surface of a pH-based enteric member has a crossing dimension perpendicular to the largest crossing dimension of less than about 15 mm, such as less than about any of 14 mm, 13 mm, 12 mm, 11 mm, 10 mm, 9 mm, 8 mm, 7 mm, 6 mm, 5 mm, 4 mm, 3 mm, 2 mm, or 1 mm. In some embodiments, the surf ace of a pH-based enteric member has a crossing dimension perpendicular to the largest crossing dimension of about any of 1 mm, 2 mm, 3 mm, 4 mm, 5 mm, 6 mm, 7 mm, 8 mm, 9 mm, 10 mm, 11 mm, 12 mm, 13 mm, 14 mm, or 15 mm.

In some embodiments, the pH-based enteric member layer, has a thickness of about 0.1 mm to about 5 mm, such as any of about 0.2 mm to about 2 mm, about 0.5 mm to about 1.5 mm, or about 0.8 mm to about 1.4 mm. In some embodiments, the pH-based enteric member layer has a thickness of at least about 0.1 mm, such as at least about any of 0.2 mm, 0.3 mm, 0.4 mm, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5 mm. In some embodiments, the pH-based enteric layer has a thickness of less than about 5 mm, such as less than about any of 4.8 mm, 4.6 mm, 4.4 mm, 4.2 mm, 4.0 mm, 3.8 mm, 3.6 mm, 3.4 mm, 3.2 mm, 3.0 mm, 2.8 mm, 2.6 mm, 2.4 mm, 2.2 mm, 2.0 mm, 1.9 mm, 1 0.8 mm, 1.7 mm, 1.6 mm, 1.5 mm, 1.4 mm, 1.3 mm, 1.2 mm, 1.1 mm, 1.0 mm, 0.9 mm, 0.8 mm, 0.7 mm, 0.6 mm, 0.5 mm, 0.4 mm, 0.3 mm, 0.2 mm, or 0.1 mm. In some embodiments, the pH-based enteric member layer has a thickness of about any of 0.1 mm, 0.2 mm, 0.3 mm, 0.4 mm, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5 mm.

In some embodiments, the pH-based enteric member layer comprises a top surface and a bottom surface, wherein the thickness, as measured between the top surface and the bottom surface, is substantially consistent, such as within a 20% range of an aver age thickness.

In some embodiments, the pH-based enteric member, such as a pH-based enteric member layer, comprises a thermoplastic material, such as a thermoplastic polymer. In some embodiments, the pH-based enteric member, such as a pH-based enteric member layer, comprises a material comprising any one or more of a matrix material, a plasticizer, or another additive, e.g., a filler, a binder, a lubricant, a glidant, and a disintegrant.

In some embodiments, the pH-based enteric member comprises one or more of: stearic acid, copolyvidone, polyvinylpyrrolidone-co-vinyl-acetate, polyvinylpyrrolidone-polyvinyl acetate copolymer, crospovidone, polyvinylpyrrolidone, hydroxypropyl cellulose, hydroxylpropyl methylcellulose or hypromellose, hydroxypropyl methylcellulose phthalate, methyl cellulose, methacrylic acid copolymer, poly (methacrylic acid-co-ethyl acrylate), poly (butyl methacrylate-co-(2-dimethylaminoethyl) methacrylate-co-methyl methacrylate), poly (dimethylaminoethylmethacrylate-co-methacrylic esters), poly (ethyl acrylate-co-methyl methacrylate-co-trimethylammonioethyl methacrylate chloride), poly (methyl acrylate-co-methyl methacrylate-co-methacrylic acid), poly (methacrylic acid-co-methylmethacrylate), poly (methacylic acid-co-ethyl acrylate), poly (methacylic acid-co-methyl methacrylate), polyethylene oxide, polyethylene glycol, polyvinyl caprolactam-polyvinyl acetate-polyethylene glycol graft copolymer, polyethylene glycol-polyvinyl alcohol graft copolymer, Kollicoat IR-polyvinyl alcohol, polyvinyl alcohol, aminoalkyl methacrylate copolymer E, hydroxypropyl methylcellulose acetate succinate or hypromellose acetate succinate, methacrylic ester copolymer, ammonioalkyl methacrylate copolymer, ethyl cellulose, polyvinyl acetate and polyvinylpyrrolidone, poly vinyl acetal diethyl aminolactate, polyvinyl acetal diethyl amino lactate, maltitol, lactose monohydrate, xylitol, isomalt, sucrose, glucose, dextran 70, or maltose.

In some embodiments, the plasticizer is any one or more of triethyl citrate, vitamine polyethylene glycol succinate, acetylated triethyl citrate, tributyl citrate, tributyl o-acetylcitrate, polyoxyl 15 hydroxystearate, peg-40 hydrogenated castor oil, polyoxyl 35 castor oil, dibutyl sebacate, diethylphthalate, glycerine, methyl 4-hydroxybenzoate, castor oil, oleic acid, triacetin, polyalkylene glycol, stearic acid, palmitic acid, malic acid, polyethylene glycol, lauroyl PEG-32 glycerides, poloxamer 188, poloxamer 407, acetyltributyl citrate, D-sorbitol, propylene glycol, diethyl phthalate, citric acid, glyceryl behenate, D-mannitol, polysorbate, sorbitan monostearate, sorbitan monooleate, or polyoxyl 40 stearate.

In some embodiments, the other additive is any one or more of acacia, alginate, alginic acid, aluminum acetate, butylparaben, butylated hydroxytoluene, citric acid, calcium carbonate, candelilla wax, croscarmellose sodium, confectioner sugar, colloidal silicone dioxide, cellulose, plain or anhydrous calcium phosphate, carnuba wax, corn starch, carboxymethylcellulose calcium, calcium disodium ethylenediaminetetraacetic acid, calcium hydrogen phosphate dehydrate, cetylpyridine chloride, calcium phosphate dibasic, calcium phosphate tribasic, dibasic calcium phosphate, disodium hydrogen phosphate, dimethicone, erythrosine sodium, ethylenediaminetetraacetic acid, gelatin, glyceryl monooleate, iron oxide, ferric oxide, iron oxide yellow, iron oxide red, lactose (hydrous, anhydrous, monohydrate, or spray dried), microcrystalline cellulose, magnesium carbonate, magnesium oxide, methyl paraben, polysorbate 80, propylene paraben, potassium bicarbonate, potassium sorbate, potato starch, phosphoric acid, polyoxyethylene (40) stearate, sodium starch glycolate, starch pregelatinized, sodium crossmellose, sodium lauryl sulfate, starch, silicon dioxide, sodium benzoate, sucrose, sorbic acid, sodium carbonate, saccharin sodium, sodium alginate, silica gel, sorbitan monooleate, sodium chloride, sodium metabisulfite, sodium citrate dehydrate, sodium starch, sodium carboxy methyl cellulose, succinic acid, sodium propionate, titanium dioxide, or talc.

In some embodiments, wherein the delay component does not comprise an erodible delay member (e.g., the delay component consists essentially of a pH-based enteric member), the erodible material of the pH-based enteric member comprises one or more of a hypromellose acetate succinate, hydroxypropyl methyl cellulose phthalate, cellulose acetate propionate (CAP), poly (methacrylic acid-co-ethyl acrylate), or polyvinyl acetate phthalate (PVAP). In some embodiments, the hypromellose acetate succinate is HPMCAS LG/LF, HPMCAS MG/MF, or HPMCAS HG/LF. In some embodiments, the hydroxypropyl methyl cellulose phthalate is HPMCP HP-50 or HPMCP HP-55. In some embodiments, the poly (methacrylic acid-co-ethyl acrylate) is Eudragit L100-55 or Eudragit L100.

ii. Erodible Delay Members

In certain embodiments, the oral drug dosage forms provided herein comprise an erodible delay member comprising an erodible material, such as different from or the same as the erodible material of a drug component. Generally, the erodible delay member is situated in an oral drug dosage form such that the erodible delay member is not subjected to a bodily fluid following administration to an individual until after erosion, such as substantially complete erosion, of a pH-based enteric member. In some embodiments, the delay components described herein comp rise an erodible delay member, such as an erodible delay member layer. In some embodiments, the erodible delay member comprises more than one layer of an erodible delay material, such as two layers of an erodible delay material. In some embodiments, the oral drug dosage form comprises one or more erodible delay members, wherein the one or more erodible delay members may be the same or different as compared to one another, such as having the same or different shape, size, and/or constitution.

In some embodiments, the erodible delay member surrounds the drug component. In such embodiments, it is possible to not have or have additional material between the erodible delay member and the drug component.

The erodible delay member is configured to erode over a predetermined amount of time following exposure to a bodily fluid, such as following erosion, such as substantially complete erosion, of a pH-based enteric member or a layer thereof. In some embodiments, the erodible delay member erodes, such as substantially erodes to expose another component of an oral drug dosage form, over a period of about 1 minute to about 7 hours. In some embodiments, the erodible delay member erodes, such as substantially erodes to expose another component of an oral drug dosage form, over a period of at least about 5 minutes, such as at least about any of 10 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 5.5 hours, 6 hours, 6.5 hours, or 7 hours. The timing of erosion of the erodible delay member, or a layer thereof, may be based on many factors including composition and thickness.

The erodible delay member may be formed using numerous materials having varying shapes and sizes. In some embodiments, the erodible delay member is a layer of an erodible delay material. In some embodiments, the erodible delay members are configured to have surfaces, such as a surface exposed to a bodily fluid during administration of the oral drug dosage form to a human individual, having a pre-determined shape and surface area. For example, in some embodiments, the erodible delay member has a top surface and a bottom surface, wherein the top surface is exposed to a bodily fluid prior to the bottom surface. In some embodiments, the erodible delay member is a layer having a top surface and a bottom surface. In some embodiments, the top surface of the erodible delay member is not flat, e.g., comprises certain features that extend beyond a top surface plane or surface tolerance threshold (as measured between two parallel planes). In some embodiments, the top surface of the erodible delay member, or at least a portion thereof, is flat or within a surface tolerance threshold.

The surfaces of an erodible delay member, such as an erodible delay member layer, as based on the surface exposed to a bodily fluid upon administration, can have any shape. In some embodiments, the surface has the shape of a capsule, circle, oval, bullet shape, arrow head shape, triangle, arced triangle, square, arced square, rectangle, arced rectangle, diamond, pentagon, hexagon, octagon, half moon, almond, or a combination thereof.

In some embodiments, the surface of an erodible delay member, such as an erodible delay member layer, has a surface area of about 10 mm² to about 400 mm², such as any of about 20 mm² to about 200 mm², about 20 mm² to about 100 mm², about 20 mm² to about 60 mm², about 30 mm² to about 50 mm². In some embodiments, the surface of an erodible delay member has a surface area of at least about 20 mm², such as at least about any of 22 mm², 24 mm², 26 mm², 28 mm², 30 mm², 32 mm², 33 mm², 34 mm², 36 mm², 38 mm², 40 mm², 42 mm², 44 mm², 46 mm², 48 mm², 50 mm², 52 mm², 54 mm², 56 mm², 58 mm², 60 mm², 65 mm², 70 mm², 80 mm², 85 mm², 90 mm², 95 mm², 100 mm², 110 mm², 120 mm², 130 mm², 140 mm², 150 mm², 160 mm², 170 mm², 180 mm², 190 mm², 200 mm², 225 mm², 250 mm², 275 mm², 300 mm², 325 mm², 350 mm², 375 mm², or 400 mm². In some embodiments, the surface of an erodible delay member has a surface area of less than about 400 mm², such as less than about any of 400 mm², 375 mm², 350 mm², 325 mm², 300 mm², 275 mm², 250 mm², 225 mm², 200 mm², 190 mm², 180 mm², 170 mm², 160 mm², 150 mm², 140 mm², 130 mm², 120 mm², 110 mm², 100 mm², 95 mm², 90 mm², 85 mm², 80 mm², 75 mm², 70 mm², 65 mm², 60 mm², 58 mm², 56 mm², 54 mm², 52 mm², 50 mm², 48 mm², 46 mm², 44 mm², 42 mm², 40 mm², 38 mm², 36 mm², 34 mm², 32 mm², 30 mm², 28 mm², 26 mm², 24 mm², 22 mm², or 20 mm². In some embodiments, the surface of an erodible delay member has a surface area of about any of 20 mm², 21 mm², 22 mm², 23 mm², 24 mm², 25 mm², 26 mm², 27 mm², 28 mm², 29 mm², 30 mm², 31 mm², 32 mm², 33 mm², 34 mm², 35 mm², 36 mm², 37 mm², 38 mm², 39 mm², 40 mm², 41 mm², 42 mm², 43 mm², 44 mm², 45 mm², 46 mm², 47 mm², 48 mm², 49 mm², 50 mm², 51 mm², 52 mm², 53 mm², 54 mm², 55 mm², 56 mm², 57 mm², 58 mm², 59 mm², 60 mm², 65 mm², 70 mm², 80 mm², 85 mm², 90 mm², 95 mm², 100 mm², 110 mm², 120 mm², 130 mm², 140 mm² 150 mm², 160 mm², 170 mm², 180 mm², 190 mm², 200 mm², 225 mm², 250 mm², 275 mm², 300 mm², 325 mm², 350 mm², 375 mm², or 400 mm².

In some embodiments, the surface area of a surface of an erodible delay member exposed to a bodily fluid is consistent throughout the thickness of the erodible delay member, e.g., as an erodible delay member erodes the surface exposed to the bodily fluid has the same surface area. In some embodiments, the surface area of a surface of an erodible delay member exposed to a bodily fluid is different at two or more points, e.g., as an erodible delay member erodes the surface exposed to the bodily fluid changes such as increases and/or decreases in surface area during erosion of the erodible delay member. In some embodiments, the shape of the surface of an erodible delay member exposed to a bodily fluid is consistent throughout the thickness of the erodible delay member. In some embodiments, the shape of a surface of an erodible delay member exposed to a bodily fluid is different at two or more points.

In some embodiments, the surface of an erodible delay member, such as an erodible delay member layer, has a largest crossing dimension of about 5 mm to about 20 mm, such as any of about 5 mm to about 15 mm, about 6 mm to about 13 mm, or about 7 to about 11 mm. In some embodiments, the sur face of an erodible delay member has a largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the surface of an erodible delay member has a largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the surface of an erodible delay member has a largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

In some embodiments, the surface of an erodible delay member, such as an erodible delay member layer, has a crossing dimension perpendicular to the largest crossing dimension of about 1 mm to about 15 mm, such as any of about 2 mm to about 10 mm, about 2 mm to about 6 mm, or about 1 to ab out 5 mm. In some embodiments, the surface of an erodible delay member has a crossing dimension perpendicular to the largest crossing dimension of at least about 1 mm, such as at least about any of 2 mm, 3 mm, 4 mm, 5 mm, 6 mm, 7 mm, 8 mm, 9 mm, 10 mm, 11 mm, 12 mm, 13 mm, 14 mm, or 15 mm. In some embodiments, the surface of an erodible delay member has a crossing dimension perpendicular to the largest crossing dimension of less than about 15 mm, such as less than about any of 14 mm, 13 mm, 12 mm, 11 mm, 10 mm, 9 mm, 8 mm, 7 mm, 6 mm, 5 mm, 4 mm, 3 mm, 2 mm, or 1 mm. In some embodiments, the surface of an erodible delay member has a crossing dimension perpendicular to the largest crossing dimension of about any of 1 mm, 2 mm, 3 mm, 4 mm, 5 mm, 6 mm, 7 mm, 8 mm, 9 mm, 10 mm, 11 mm, 12 mm, 13 mm, 14 mm, or 15 mm.

In some embodiments, the erodible delay member layer, has a thickness of about 0.1 mm to about 5 mm, such as any of about 0.2 mm to about 2 mm, about 0.5 mm to about 1.5 mm, about 0.6 mm to about 1.6 mm, or about 0.8 mm to about 1.4 mm. In some embodiments, the erodible delay member layer has a thickness of at least about 0.1 mm, such as at least about any of 0.2 mm, 0.3 mm, 0.4 mm, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5 mm. In some embodiments, the erodible delay layer has a thickness of less than about 5 mm, such as less than about any of 4.8 mm, 4.6 mm, 4.4 mm, 4.2 mm, 4.0 mm, 3.8 mm, 3.6 mm, 3.4 mm, 3.2 mm, 3.0 mm, 2.8 mm, 2.6 mm, 2.4 mm, 2.2 mm, 2.0 mm, 1.9 mm, 1.8 mm, 1.7 mm, 1.6 mm, 1.5 mm, 1.4 mm, 1.3 mm, 1.2 mm, 1.1 mm, 1.0 mm, 0.9 mm, 0.8 mm, 0.7 mm, 0.6 mm, 0.5 mm, 0.4 mm, 0.3 mm, 0.2 mm, or 0.1 mm. In some embodiments, the erodible delay layer has a thickness of about any of 0.1 mm, 0.2 mm, 0.3 mm, 0.4 mm, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5 mm. In some embodiments, the erodible delay layer has a thickness of about any of 0.6 mm, 0.7 mm, 0.8 mm, 0.9 mm, 1.1 mm, 1.2 mm, 1.3 mm, 1.4 mm, 1.5 mm, or 1.6 mm. The inventors of the present disclosure found that when the thickness of the delayed layer is thick, the surface of the delayed layer is prone to gelation, and it is easy to pass through the gastrointestinal tract without releasing the drug in the colon, while when the thickness of the delayed layer is thin, some surfaces of the delayed layer are prone to damage and release the drug in advance due to the uneven fluid flow. Therefore, it is necessary to make a delay layer of the right thickness to ensure that the drug is released in the colon without gelation of the delayed layer, and that it is not affected by body fluids to release the drug in advance.

In some embodiments, the erodible delay member layer comprises a top surface and a bottom surface, wherein the thickness, as measured between the top sur face and the bottom surface, is substantially consistent, such as within a 20% range of an average thickness.

In some embodiments, the erodible delay member, such as an erodible delay member layer, comprises a thermoplastic material, such as a thermoplastic polymer. In some embodiments, the erodible delay member, such as an erodible delay member layer, comprises a material comprising any one or more of a matrix material, a plasticizer, or another additive, e.g., a filler, a binder, a lubricant, a glidant, an inorganic salt and a disintegrant. The inventors of the present disclosure found that the erodible delay layer of the present disclosure is capable of constituting erosion, e.g., erodes layer by layer, without gelation resulting in delayed drug release.

In some embodiments, the thermoplastic polymer comprises one or more of stearic acid, medium chain triglycerides, glyceryl distearate, propylene glycol monolaurate, propylene glycol caprylate, oleoyl polyoxyl-6 glycerides, PEG-6 STEARATE, PEG-32 STEARATE, linoleoyl polyoxyl-6 glycerides, lauroyl polyoxyl-32 glycerides, caprylocaproyl polyoxyl-8 glycerides, sorbitan monolaurate, sorbitan monopalmitate, sorbitan monostearate, sorbitan oleate, glyceryl monolinoleate, copolyvidone, polyvinylpyrrolidone-co-vinyl-acetate, polyvinylpyrrolidone-polyvinyl acetate copolymer, crospovidone, polyvinylpyrrolidone, hydroxypropyl cellulose, hydroxylpropyl methylcellulose or Hypromellose, hydroxypropyl methylcellulose phthalate, methyl cellulose, methacrylic acid copolymer, poly (methacrylic acid-co-ethyl acrylate), poly (butyl methacrylate-co-(2-dimethylaminoethyl) methacrylate-co-methyl methacrylate), poly (dimethylaminoethylmethacrylate-co-methacrylic esters), poly (ethyl acrylate-co-methyl methacrylate-co-trimethylammonioethyl methacrylate chloride), poly (methyl acrylate-co-methyl methacrylate-co-methacrylic acid), poly (methacrylic acid-co-methylmethacrylate), poly (methacylic acid-co-ethyl acrylate), poly (methacylic acid-co-methyl methacrylate), polyethylene oxide, polyethylene glycol, polyvinyl caprolactam-polyvinyl acetate-polyethylene glycol graft copolymer, polyethylene glycol-polyvinyl alcohol graft copolymer, Kollicoat IR-polyvinyl alcohol, polyvinyl alcohol, aminoalkyl methacrylate copolymer E, hydroxypropyl methylcellulose acetate succinate or hypromellose acetate succinate, methacrylic ester copolymer, ammonioalkyl methacrylate copolymer, ethyl cellulose, polyvinyl acetate and polyvinylpyrrolidone, polyvinyl acetal diethyl aminolactate, polyvinyl acetal diethyl amino lactate, maltitol, lactose monohydrate, xylitol, isomalt, sucrose, glucose, dextran 70, maltose, Lauroyl Polyoxyl-32 glycerides, caprylocaproyl polyoxyl-8 glycerides, polyoxyl (35) Castor oil, Vitamin E polyethylene glycol succinate, PEG-40 hydrogenated Castor oil, glyceryl monolinoleate, or glyceryl dibehenate. In some embodiments, the thermoplastic polymer comprises one or more of hydroxypropyl cellulose and Vitamin E polyethylene glycol succinate. In some embodiments, the weight of the thermoplastic polymer is 60-85% of the weight of the erodible delay member.

In some embodiments, the plasticizer is any one or more of triethyl citrate, vitamine polyethylene glycol succinate, acetylated triethyl citrate, tributyl citrate, tributyl o-acetylcitrate, polyoxyl 15 hydroxystearate, peg-40 hydrogenated castor oil, polyoxyl 35 castor oil, dibutyl sebacate, diethylphthalate, glycerine, methyl 4-hydroxybenzoate, castor oil, oleic acid, triacetin, polyalkylene glycol, stearic acid, palmitic acid, malic acid, polyethylene glycol, lauroyl PEG-32 glycerides, poloxamer 188, poloxamer 407, acetyltributyl citrate, D-sorbitol, propylene glycol, diethyl phthalate, citric acid, glyceryl behenate, D-mannitol, polysorbate, sorbitan monostearate, sorbitan monooleate, or polyoxyl 40 stearate. In some embodiments, the plasticizer is triethyl citrate. In some embodiments, the weight of the plasticizer is 5-20% of the weight of the erodible delay member. In some embodiments, the weight of the plasticizer is 10-15% of the weight of the erodible delay member.

In some embodiments, the inorganic salt is any one or more of calcium phosphate, calcium hydrogen phosphate, disodium hydrogen phosphate, sodium carbonate, sodium chloride, sodium phosphate, calcium hydro gen phosphate, calcium carbonate, calcium chloride, potassium phosphate, dipotassium hydrogen phosphate, potassium carbonate and potassium chloride. In some embodiments, the inorganic salt is calcium hydrogen phosphate. In some embodiments, the weight of the inorganic salt is 5-20% of the weight of the erodible delay member. In some embodiments, the weight of the inorganic salt is 10% of the weight of the erodible delay member.

In some embodiments, the other additive is any one or more of acacia, alginate, alginic acid, aluminum acetate, butylparaben, butylated hydroxytoluene, citric acid, calcium carbonate, candelilla wax, croscarmellose sodium, confectioner sugar, colloidal silicone dioxide, cellulose, plain or anhydrous calcium phosphate, carnuba wax, corn starch, carboxymethylcellulose calcium, calcium disodium ethylenediaminetetraacetic acid, calcium hydrogen phosphate dehydrate, cetylpyridine chloride, calcium hydrophosphate, calcium phosphate dibasic, calcium phosphate tribasic, dibasic calcium phosphate, disodium hydrogen phosphate, dimethicone, erythrosine sodium, ethylenediaminetetraacetic acid, gelatin, glyceryl monooleate, iron oxide, ferric oxide, iron oxide yellow, iron oxide red, lactose (hydrous, anhydrous, monohydrate, or spray dried), microcrystalline cellulose, magnesium carbonate, magnesium oxide, methyl paraben, polysorbate 80, propylene paraben, potassium bicarbonate, potassium sorbate, potato starch, phosphoric acid, polyoxyethylene (40) stearate, sodium starch glycolate, starch pregelatinized, sodium crossmellose, sodium lauryl sulfate, starch, silicon dioxide, sodium benzoate, sucrose, sorbic acid, sodium carbonate, saccharin sodium, sodium alginate, silica gel, sorbitan monooleate, sodium chloride, sod ium metabisulfite, sodium citrate dehydrate, sodium starch, sodium carboxy methyl cellulose, succinic acid, sodium propionate, titanium dioxide, or talc.

iii. Bacterial Degradation Moiety and Bacterial Degradation Members

In certain embodiments, the oral drug dosage forms provided herein comprise a bacterial degradation moiety. As described herein, a bacterial degradation moiety is a moiety that can be digested by one or more bacteria present, or likely to be present, in the colon of an individual. Such moieties can aid in the colon-specific breakdown of one or more aspects of an oral drug dosage form. In some embodiments, the bacterial degradation moiety is admixed in an aspect of an oral drug dosage form, such as an erodible delay member or a drug component. In some embodiments, the delay component further comprises a bacterial degradation member comprising an erodible material admixed with the bacterial degradation moiety. In some embodiments wherein the oral drug dosage form comprises a bacterial degradation member, the oral drug dosage form is configured such that the pH-based enteric member erodes prior to the erodible delay member, the erodible delay member erodes prior to the bacterial degradation member, and the bacterial degradation member erodes prior to the drug component. In some embodiments wherein the oral drug dosage form comprises a bacterial degradation member, the pH-based enteric member prevents erosion, at least in part, of the erodible delay member, the erodible delay member prevents erosion, at least in part, of the bacterial degradation member, and the bacterial degradation member prevents erosion, at least in part, of the drug component. In some embodiments, the oral drug dosage form comprises one or more bacterial degradation members, wherein the one or more bacterial degradation members may be the same or different as compared to one another, such as having the same or different shape, size, and/or constitution.

In some embodiments, the bacterial degradation moiety is digestible by one or more microorganisms present in the colon of the individual. In some embodiments, the bacterial degradation moiety comprises one or more saccharides. In some embodiments, the one or more saccharides comprises any one or more of a sucrose, glucose, xylose, fructose, maltose, galactose pectin, galactomannan, dextran, inulin, chitosan, carrageenan, cellulose acetate propionate (CAP), peptidoglycan, gellan, xanthan gum, lentinan, psyllium polysaccharide, maize bran arabinoxylan, alginate, hyaluronic acid, fucoidan, shellac, agar, or maltodextrin.

In some embodiments, the oral drug dosage form comprises an amount of the bacterial degradation moiety of about 0.5 mg to about 500 mg. In some embodiments, the amount of a bacterial degradation moiety in an oral drug dosage form is about 1 mg or more, such as about any of 5 mg or more, 10 mg or more, 15 mg or more, 20 mg or more, 25 mg or more, 30 mg or more, 35 mg or more, 40 mg or more, 45 mg or more, 50 mg or more, 55 mg or more, 60 mg or more, 65 mg or more, 70 mg or more, 75 mg or more, 80 mg or more, 85 mg or more, 90 mg or more, 95 mg or more, 100 mg or more, 110 mg or more, 120 mg or more, 130 mg or more, 140 mg or more, 150 mg or more, 160 mg or more, 170 mg or more, 180 mg or more, 190 mg or more, 200 mg or more, 210 mg or more, 220 mg or more, 230 mg or more, 240 mg or more, 250 mg or more, 260 mg or more, 270 mg or more, 280 mg or more, 290 mg or more, 300 mg or more, 310 mg or more, 320 mg or more, 330 mg or more, 340 mg or more, 350 mg or more, 360 mg or more, 370 mg or more, 380 mg or more, 390 mg or more, 400 mg or more, 410 mg or more, 420 mg or more, 430 mg or more, 440 mg or more, 450 mg or more, 460 mg or more, 470 mg or more, 480 mg or more, 490 mg or more, or 500 mg or more. In some embodiments, the amount of a bacterial degradation moiety in an oral drug dosage form is about 500 mg or less, such as about any of 490 mg or less, 480 mg or less, 470 mg or less, 460 mg or less, 450 mg or less, 440 mg or less, 430 mg or less, 420 mg or less, 410 mg or less, 400 mg or less, 390 mg or less, 380 mg or less, 370 mg or less, 360 mg or less, 350 mg or less, 340 mg or less, 330 mg or less, 320 mg or less, 310 mg or less, 300 mg or less, 290 mg or less, 280 mg or less, 270 mg or less, 260 mg or less, 250 mg or less, 240 mg or less, 230 mg or less, 220 mg or less, 210 mg or less, 200 mg or less, 190 mg or less, 180 mg or less, 170 mg or less, 160 mg or less, 150 mg or less, 140 mg or less, 130 mg or less, 120 mg or less, 110 mg or less, 100 mg or less, 90 mg or less, 80 mg or less, 70 mg or less, 60 mg or less, 50 mg or less, 40 mg or less, 30 mg or less, 20 mg or less, 10 mg or less, 5 mg or less, or 1 mg or less. In some embodiments, the amount of a bacterial degradation moiety in an oral drug dosage form is about any of 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, or 500 mg.

In some embodiments, the bacterial degradation member is configured to erode over (including within) a predetermined amount of time following exposure to a bodily fluid, such as following erosion, such as substantially complete erosion, of a pH-based enteric member or a layer thereof and/or an erodible delay member or a layer thereof. In some embodiments, the bacterial degradation member erodes, such as substantially erodes to expose another component of an oral drug dosage form over a period of about 1 minute to about 7 hours. In some embodiments, the bacterial degradation member erodes, such as substantially erodes to expose another component of an oral drug dosage form, over a period of at least about 5 minutes, such as at least about any of 10 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 5.5 hours, 6 hours, 6.5 hours, or 7 hours. The timing of erosion of the bacterial degradation member, or a layer thereof, may be based on many factors including composition (e.g., type and/or amount of a bacterial degradation moiety) and thickness.

The bacterial degradation member may be formed using numerous materials having varying shapes and sizes. In some embodiments, the bacterial degradation member is a layer of a bacterial degradation material. In some embodiments, the bacterial degradation members are configured to have surfaces, such as a surface exposed to a bodily fluid during administration of the oral drug dosage form to a human individual, having a pre-determined shape and surface area. For example, in some embodiments, the bacterial degradation member has a top surface and a bottom surface, wherein the top surface is exposed to a bodily fluid prior to the bottom surface. In some embodiments, the bacterial degradation member is a layer having a top surface and a bottom surface. In some embodiments, the top surface of the bacterial degradation member is not flat, e.g., comprises certain features that extend beyond a top surface plane or surface tolerance threshold (as measured between two parallel planes). In some embodiments, the top surface of the bacterial degradation member, or at least a portion thereof, is flat or within a surface tolerance threshold.

In some embodiments, the bacterial degradation member surrounds the drug component. In such embodiments, it is possible to not have or have additional material between the bacterial degradation member and the drug component, e.g., the presence of an erodible delay member.

The surfaces of a bacterial degradation member, such as a bacterial degradation member layer, as based on the surface exposed to a bodily fluid upon administration, can have any shape. In some embodiments, the surface has the shape of a capsule, circle, oval, bullet shape, arrow head shape, triangle, arced triangle, square, arced square, rectangle, arced rectangle, diamond, pentagon, hexagon, octagon, half moon, almond, or a combination thereof.

In some embodiments, the surface of a bacterial degradation member, such as a bacterial degradation member layer, has a surface area of about 10 mm² to about 400 mm², such as any of about 20 mm² to about 200 mm², about 20 mm² to about 100 mm², about 20 mm² to about 60 mm², about 30 mm² to about 50 mm². In some embodiments, the surface of a bacterial degradation member has a surface area of at least about 20 mm², such as at least about any of 22 mm², 24 mm², 26 mm², 28 mm², 30 mm², 32 mm², 33 mm², 34 mm², 36 mm², 38 mm², 40 mm², 42 mm², 44 mm², 46 mm², 48 mm², 50 mm², 52 mm², 54 mm², 56 mm², 58 mm², 60 mm², 65 mm², 70 mm², 80 mm², 85 mm², 90 mm², 95 mm², 100 mm², 110 mm², 120 mm², 130 mm², 140 mm², 150 mm², 160 mm², 170 mm², 180 mm², 190 mm², 200 mm², 225 mm², 250 mm², 275 mm², 300 mm², 325 mm², 350 mm², 375 mm², or 400 mm². In some embodiments, the surface of a bacterial degradation member has a surface area of less than about 400 mm², such as less than about any of 400 mm², 375 mm², 350 mm², 325 mm², 300 mm², 275 mm², 250 mm², 225 mm², 200 mm², 190 mm², 180 mm², 170 mm², 160 mm², 150 mm², 140 mm², 130 mm², 120 mm², 110 mm², 100 mm², 95 mm², 90 mm², 85 mm², 80 mm², 75 mm², 70 mm², 65 mm², 60 mm², 58 mm², 56 mm², 54 mm², 52 mm², 50 mm², 48 mm², 46 mm², 44 mm², 42 mm², 40 mm², 38 mm², 36 mm², 34 mm², 32 mm², 30 mm², 28 mm², 26 mm², 24 mm², 22 mm², or 20 mm². In some embodiments, the surface of a bacterial degradation member has a surface area of about any of 20 mm², 21 mm², 22 mm², 23 mm², 24 mm², 25 mm², 26 mm², 27 mm², 28 mm², 29 mm², 30 mm², 31 mm², 32 mm², 33 mm², 34 mm², 35 mm², 36 mm², 37 mm², 38 mm², 39 mm², 40 mm², 41 mm², 42 mm², 43 mm², 44 mm², 45 mm², 46 mm², 47 mm², 48 mm², 49 mm², 50 mm², 51 mm², 52 mm², 53 mm², 54 mm², 55 mm², 56 mm², 57 mm², 58 mm², 59 mm², 60 mm², 65 mm², 70 mm², 80 mm², 85 mm², 90 mm², 95 mm², 100 mm² 110 mm², 120 mm², 130 mm², 140 mm², 150 mm², 160 mm², 170 mm², 180 mm² 190 mm², 200 mm², 225 mm², 250 mm², 275 mm², 300 mm², 325 mm², 350 mm² 375 mm², or 400 mm².

In some embodiments, the surface area of a surface of a bacterial degradation member exposed to a bodily fluid is consistent throughout the thickness of the bacterial degradation member, e.g., as a bacterial degradation member erodes the surface exposed to the bodily fluid has the same surface area. In some embodiments, the surface area of a surface of a bacterial degradation member exposed to a bodily fluid is different at two or more points, e.g., as a bacterial degradation member erodes the surface exposed to the bodily fluid changes such as increases and/or decreases in surface area during erosion of the bacterial degradation member. In some embodiments, the shape of the surface of a bacterial degradation member exposed to a bodily fluid is consistent throughout the thickness of the bacterial degradation member. In some embodiments, the shape of a surface of a bacterial degradation member exposed to a bodily fluid is different at two or more points.

In some embodiments, the surface of a bacterial degradation member, such as a bacterial degradation member layer, has a largest crossing dimension of about 5 mm to about 20 mm, such as any of about 5 mm to about 15 mm, ab out 6 mm to about 13 mm, or about 7 to about 11 mm. In some embodiments, the surface of a bacterial degradation member has a largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the surface of a bacterial degradation member has a largest crossing dimension of less than ab out 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the surface of a bacterial degradation member has a largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

In some embodiments, the surface of a bacterial degradation member, such as a bacterial degradation member layer, has a crossing dimension perpendicular to the largest crossing dimension of about 1 mm to about 15 mm, such as any of about 2 mm to about 10 mm, about 2 mm to about 6 mm, or about 1 to about 5 mm. In some embodiments, the surface of a bacterial degradation member has a crossing dimension perpendicular to the largest crossing dimension of at least about 1 mm, such as at least about any of 2 mm, 3 mm, 4 mm, 5 mm, 6 mm, 7 mm, 8 mm, 9 mm, 10 mm, 11 mm, 12 mm, 13 mm, 14 mm, or 15 mm. In some embodiments, the surface of a bacterial degradation member has a crossing dimension perpendicular to the largest crossing dimension of less than about 15 mm, such as less than about any of 14 mm, 13 mm, 12 mm, 11 mm, 10 mm, 9 mm, 8 mm, 7 mm, 6 mm, 5 mm, 4 mm, 3 mm, 2 mm, or 1 mm. In some embodiments, the surface of a bacterial degradation member has a crossing dimension perpendicular to the largest crossing dimension of about any of 1 mm, 2 mm, 3 mm, 4 mm, 5 mm, 6 mm, 7 mm, 8 mm, 9 mm, 10 mm, 11 mm, 12 mm, 13 mm, 14 mm, or 15 mm.

In some embodiments, the bacterial degradation member layer, has a thickness of about 0.1 mm to about 5 mm, such as any of about 0.2 mm to about 2 mm, about 0.5 mm to about 1.5 mm, or about 0.8 mm to about 1.4 mm. In some embodiments, the bacterial degradation member layer has a thickness of at least about 0.1 mm, such as at least about any of 0.2 mm, 0.3 mm, 0.4 mm, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5 mm. In some embodiment s, the bacterial degradation member layer has a thickness of less than about 5 mm, such as less than about any of 4.8 mm, 4.6 mm, 4.4 mm, 4.2 mm, 4.0 mm, 3.8 mm, 3.6 mm, 3.4 mm, 3.2 mm, 3.0 mm, 2.8 mm, 2.6 mm, 2.4 mm, 2.2 mm, 2.0 mm, 1.9 mm, 1.8 mm, 1.7 mm, 1.6 mm, 1.5 mm, 1.4 mm, 1.3 mm, 1.2 mm, 1.1 mm, 1.0 mm, 0.9 mm, 0.8 mm, 0.7 mm, 0.6 mm, 0.5 mm, 0.4 mm, 0.3 mm, 0.2 mm, or 0.1 mm. In some embodiments, the bacterial degradation member layer has a thickness of about any of 0.1 mm, 0.2 mm, 0.3 mm, 0.4 mm, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5 mm.

In some embodiments, the bacterial degradation member layer comprises a top surface and a bottom surface, wherein the thickness, as measured between the top surface and the bottom surface, is substantially consistent, such as within a 20% range of an average thickness.

In some embodiments, the bacterial degradation member, such as a bacterial degradation member layer, comprises a thermoplastic material, such as a thermo plastic polymer. In some embodiments, the bacterial degradation member, such as a bacterial degradation member layer, comprises a material comprising any one or more of a matrix material, a plasticizer, or another additive, e.g., a filler a binder, a lubricant, a glidant, and a disintegrant.

In some embodiments, the bacterial degradation member comprises one or more of stearic acid, medium chain triglycerides, glyceryl distearate, propylene glycol monolaurate, propylene glycol caprylate, oleoyl polyoxyl-6 glycerides, PEG-6 STEARATE, PEG-32 STEARATE, linoleoyl polyoxyl-6 glycerides, lauroyl polyoxyl-32 glycerides, caprylocaproyl polyoxyl-8 glycerides, sorbitan monolaurate, sorbitan monopalmitate, sorbitan monostearate, sorbitan oleate, glyceryl monolinoleate, copolyvidone, polyvinylpyrrolidone-co-vinyl-acetate, polyvinylpyrrolidone-polyvinyl acetate copolymer, crospovidone, polyvinylpyrrolidone, hydroxypropyl cellulose, hydroxylpropyl methylcellulose or Hypromellose, hydroxypropyl methylcellulose phthalate, methyl cellulose, methacrylic acid copolymer, poly (methacrylic acid-co-ethyl acrylate), poly (butyl methacrylate-co-(2-dimethylaminoethyl) methacrylate-co-methyl methacrylate), poly (dimethylaminoethylmethacrylate-co-methacrylic esters), poly (ethyl acrylate-co-methyl methacrylate-co-trimethylammonioethyl methacrylate chloride), poly (methyl acrylate-co-methyl methacrylate-co-methacrylic acid), poly (methacrylic acid-co-methylmethacrylate), poly (methacylic acid-co-ethyl acrylate), poly (methacylic acid-co-methyl methacrylate), polyethylene oxide, polyethylene glycol, polyvinyl caprolactam-polyvinyl acetate-polyethylene glycol graft copolymer, polyethylene glycol-polyvinyl alcohol graft copolymer, Kollicoat JR-polyvinyl alcohol, polyvinyl alcohol, aminoalkyl methacrylate copolymer E, hydroxypropyl methylcellulose acetate succinate or hypromellose acetate succinate, methacrylic ester copolymer, ammonioalkyl methacrylate copolymer, ethyl cellulose, polyvinyl acetate and polyvinylpyrrolidone, polyvinyl acetal diethyl aminolactate, polyvinyl acetal diethyl amino lactate, maltitol, lactose monohydrate, xylitol, isomalt, sucrose, glucose, dextran 70, maltose, Lauroyl Polyoxyl-32 glycerides, caprylocaproyl polyoxyl-8 glycerides, polyoxyl (35) Castor oil, Vitamin E polyethylene glycol succinate, PEG-40 hydrogenated Castor oil, glyceryl monolinoleate, or glyceryl dibehenate.

### 4. Shells

In certain aspects, provided herein is an oral drug dosage comprising a shell.

In some embodiments, the shell comprises an insulating material that is impermeable to bodily fluids, such as gastrointestinal fluid. In some embodiments the shell comprises an insulating material that is impermeable to certain bodily fluids, such as stomach fluid. In some embodiments, the shell comprises an insulating material that is impermeable to bodily fluids at a certain pH, e.g., an insulating material that is impermeable to bodily fluids at a pH of less than about 7, such as less than about any 7.5, 8, or 8.5 (e.g., the shell does not erode until subjected to a pH of great than about 7, such as greater than about any of 7.5, 8, or 8.5). In some embodiments, the shell comprises an insulating material that is a non-erodible material, e.g., does not substantially erode during administration to a human individual. In some embodiments, the shell comprises an insulating material that is non-erodible in certain bodily fluids, such as stomach fluid.

In some embodiments, the shell comprises an insulating material that is an erodible material having a pH-sensitive erosion and/or an erosion rate that allows for the complete release of a drug from the drug component of an oral drug dosage form prior to exposure of a drug component to bodily fluids due to erosion of the shell.

In some embodiments, the shell comprises an insulating material that is selectively permeable. For example, in some embodiments, the shell is permeable to a bodily fluid, and is impermeable to a drug.

The shells provided herein are configured to have surfaces, such as an exterior facing surface exposed to a bodily fluid during administration of the oral drug dosage form to a human individual. In some embodiments, the shell is configured to control expo sure of another component of an oral drug dosage form (e.g., a pH-based enteric member and an erodible delay member) to a bodily fluid, such as to guide erosion thereof. In some embodiments, the exterior surface of the shell is not flat, e.g., comprises certain features that extend beyond a surface plane or surface tolerance threshold (as measured between two parallel planes), such as to reduce adherence of the shell, or a portion thereof, to an internal body part of the human individual. In some embodiments, the exterior surface of the shell, or at least a portion thereof, is flat or within a surface tolerance threshold.

The surfaces of the shell, as based on the surfaces exposed to a bodily fluid, can have any shape. In some embodiments, the surface of a shell, as based on the surface ex posed to a bodily fluid, has the shape of a capsule, cylinder, elliptic cylinder, circle, oval, bullet shape, arrow head shape, triangle, arced triangle, square, arced square, rectangle, arced rectangle, diamond, pentagon, hexagon, octagon, half moon, almond, or a combination thereof. In some embodiments, the shell forms a ringed shape, wherein other components of an oral drug dosage form are contained within the ringed shape (e.g., a drug component, a pH-based enteric member, and an erodible delay member).

In some embodiments, the shell has a largest crossing dimension of about 5 mm to about 20 mm, such as any of about 5 mm to about 15 mm, about 6 mm to about 13 mm, or about 7 to about 11 mm. In some embodiments, the shell has a largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the shell has a largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the shell has a largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the largest crossing dimension is measured across a surface of an oral drug dosage form.

In some embodiments, the shell has a crossing dimension perpendicular to a largest crossing dimension of about 5 mm to about 20 mm, such as any of about 5 mm to about 15 mm, about 6 mm to about 13 mm, or about 7 to about 11 mm. In some embodiments, the shell has a crossing dimension perpendicular to a largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the shell has a crossing dimension perpendicular to a largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the shell has a crossing dimension perpendicular to a largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the crossing dimension perpendicular to a largest crossing dimension is measured across a surface of an oral drug dosage form.

In some embodiments, the shell is configured to have a thickness to prevent and/or inhibit exposure of components of an oral drug dosage form, or a portion thereof, to a bodily fluid. In some embodiments, the shell has a thickness, as measured from an exterior surface of an oral drug dosage form to another component there of, of about 0.3 mm to about 3 mm, such as any of about 0.3 to about 1 mm, about 0.4 mm to about 2 mm, or about 0.5 mm to about 1.5 mm. In some embodiments, the shell has a thickness of at least about 0.3 mm, such as at least about any of 0.4, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm. In some embodiments, the shell has a thickness of less than about 3 mm, such as less than about any of 2.8 mm, 2.6 mm, 2.4 mm, 2.2 mm, 2.0 mm, 1.9 mm, 1.8 mm, 1.7 mm, 1.6 mm, 1.5 mm, 1.4 mm, 1.3 mm, 1.2 mm, 1.1 mm, 1.0 mm, 0.9 mm, 0.8 mm, 0.7 mm, 0.6 mm, 0.5 mm, 0.4 mm, or 0.3. In some embodiments, the shell has a thickness of about any of 0.3, 0.4 mm, 0.5 mm, 0.6 mm, 0.7 mm, 0.8 mm, 0.9 mm, 1.1 mm, 1.2 mm, 1 0.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm.

In some embodiments, the shell comprises a thermoplastic material, such as a thermoplastic polymer. In some embodiments, the shell comprises a material comprising any one or more of a matrix material, a plasticizer, or another additive, e.g., a filler, a binder, a lubricant, a glidant, and a disintegrant.

In some embodiments, the matrix material comprises any one or more of copolyvidone, polyvinylpyrrolidone-co-vinyl-acetate, polyvinylpyrrolidone-polyvinyl acetate copolymer, crospovidone, polyvinylpyrrolidone, hydroxypropyl cellulose, hydroxylpropyl methylcellulose or Hypromellose, hydroxypropyl methylcellulose phthalate, methyl cellulose, methacrylic acid copolymer, poly (methacrylic acid-co-ethyl acrylate), poly (butyl methacrylate-co-(2-dimethylaminoethyl) methacrylate-co-methyl methacrylate), poly (dimethylaminoethylmethacrylate-co-methacrylic esters), poly (ethyl acrylate-co-methyl methacrylate-co-trimethylammonioethyl methacrylate chloride), poly (methyl acrylate-co-methyl methacrylate-co-methacrylic acid), poly (methacrylic acid-co-methylmethacrylate), poly (methacylic acid-co-ethyl acrylate), poly (methacylic acid-co-methyl methacrylate), polyethylene oxide, polyethylene glycol, polyvinyl caprolactam-polyvinyl acetate-polyethylene glycol graft copolymer, polyethylene glycol-polyvinyl alcohol graft copolymer, Kollicoat IR-polyvinyl alcohol, polyvinyl alcohol, aminoalkyl methacrylate copolymer E, hydroxypropyl methylcellulose acetate succinate or hypromellose acetate succinate, methacrylic ester copolymer, ammonioalkyl meth acrylate copolymer, ethyl cellulose, polyvinyl acetate and polyvinylpyrrolidone, poly vinyl acetal diethyl aminolactate, polyvinyl acetal diethyl amino lactate, maltitol, lactose monohydrate, xylitol, isomalt, sucrose, glucose, dextran 70, or maltose.

In some embodiments, the plasticizer is any one or more of triethyl citrate, vitamine polyethylene glycol succinate, acetylated triethyl citrate, tributyl citrate, tributyl o-acetylcitrate, polyoxyl 15 hydroxystearate, peg-40 hydrogenated castor oil, polyoxyl 35 castor oil, dibutyl sebacate, diethylphthalate, glycerine, methyl 4-hydroxybenzoate, castor oil, oleic acid, triacetin, polyalkylene glycol, stearic acid, palmitic acid, malic acid, polyethylene glycol, lauroyl PEG-32 glycerides, poloxamer 188, poloxamer 407, acetyltributyl citrate, D-sorbitol, propylene glycol, diethyl phthalate, citric acid, glyceryl behenate, D-mannitol, polysorbate, sorbitan monostearate, sorbitan monooleate, or polyoxyl 40 stearate.

In some embodiments, the other additive is any one or more of acacia, alginate, alginic acid, aluminum acetate, butylparaben, butylated hydroxytoluene, citric acid, calcium carbonate, candelilla wax, croscarmellose sodium, confectioner sugar, colloidal silicone dioxide, cellulose, plain or anhydrous calcium phosphate, carnuba wax, corn starch, carboxymethylcellulose calcium, calcium disodium ethylenediaminetetraacetic acid, calcium hydrogen phosphate dehydrate, cetylpyridine chloride, calcium phosphate dibasic, calcium phosphate tribasic, dibasic calcium phosphate, disodium hydrogen phosphate, dimethicone, erythrosine sodium, ethylenediaminetetraacetic acid, gelatin, glyceryl monooleate, iron oxide, ferric oxide, iron oxide yellow, iron oxide red, lactose (hydrous, anhydrous, monohydrate, or spray dried), microcrystalline cellulose, magnesium carbonate, magnesium oxide, methyl paraben, polysorbate 80, propylene paraben, potassium bicarbonate, potassium sorbate, potato starch, phosphoric acid, polyoxyethylene (40) stearate, sodium starch glycolate, starch pregelatinized, sodium crossmellose, sodium lauryl sulfate, starch, silicon dioxide, sodium benzoate, sucrose, sorbic acid, sodium carbonate, saccharin sodium, sodium alginate, silica gel, sorbitan monooleate, sodium chloride, sodium metabisulfite, sodium citrate dehydrate, sodium starch, sodium carboxy methyl cellulose, succinic acid, sodium propionate, titanium dioxide, or talc.

### 5. Additional Components

In some embodiments, the oral drug dosage form comprises one or more additional components.

In some embodiments, the oral drug dosage form comprises an excipient.

In some embodiments, the excipient is admixed in the drug component. In some embodiments, the excipient is admixed in an erodible delay member. In some embodiments, the excipient is admixed in a bacterial degradation member. In some embodiments, the excipient is admixed in an excipient component, wherein the excipient component comprises an erodible material. In some embodiments, the excipient comprises one or more of an absorption enhancer, pH-lowering agent, or a disintegrant. In some embodiments, the excipient is selected based on whether the oral drug dosage form, or a drug therein, is intended for local or systemic delivery. For example, absorption enhancers may find particular utility in an oral drug dosage form formulated for system delivery. In some embodiments, the oral drug dosage form comprises a drug intended for system delivery and a drug intended for local delivery (such as arranged in different compartment of the oral drug dosage form), wherein the oral drug dosage form is configured such that an absorption enhancing excipient is released to aid in the systemic uptake of the drug intended for systemic delivery.

In some embodiments, the oral drug dosage form comprises an adjuvant. For example, certain drugs, such as biological agents, e.g., peptides, and/or nucleic acids, may benefit from an adjuvant to help protect the drug once released into the gastrointestinal tract thereby providing improved uptake. In some embodiments, the adjuvant is a protease scavenger and/or a protease inhibitor. In some embodiments, the protease scavenger or the protease inhibitor is admixed with the delay component, or a portion thereof (such as an erodible delay component). In some embodiments, the protease scavenger or the protease inhibitor is admixed in the erodible material of the erodible delay member.

In some embodiments, the additional component comprises, such as an outer coating. In some embodiments, the outer coating is a flavor coating. In some embodiments, the outer coating is a sugar coating. In some embodiments, the outer coating is a cosmetic coating. In some embodiments, the outer coating is a color coating. In some embodiments, the outer coating is a film coating. In some embodiments, the outer coating is a polymer coating. In some embodiments, the additional component is a label, such as a company name, abbreviation, or logo, a medication label or drug name, such as a drug brand name and/or drug chemical name or abbreviation, a drug amount or strength, an identification barcode, or any combination thereof.

### C. Release Profiles of Oral Drug Dosage Forms and Components Thereof

The oral drug dosage form described herein are configured to release a drug at desired location in the colon of an individual. In some embodiments, the oral drug dosage form is configured to release the drug at, near, or downstream of any one of the cecum, ascending colon, hepatic flexure, transverse colon, splenic flexure, descending colon, or sigmoid colon. In some embodiments, at least about 95% (such as at least about any of 96%, 97%, 98%, 99%, or 100%) of the drug in the oral drug dosage form is released from the oral drug dosage form in the colon of the individual. In some embodiments, less than about 2% (such as less than about any of 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, or 0.1%) of the drug in the oral drug dos age form is released from the oral drug dosage form outside of the colon of the individual. In some embodiments, substantially all, including all, of the drug in the oral drug dosage form is released from the oral drug dosage form in the colon of the individual.

In some embodiments, the oral drug dosage form is configured to release the drug at, near, or downstream of the ileum of the individual. In some embodiments, the oral drug dosage form is configured to release the drug at, near, or downstream of the large intestine (colon), or a portion thereof, of the individual. In some embodiments, the oral drug dosage form is configured to release the drug at, near, or downstream of the cecum, ascending colon, transverse colon, descending colon, and/or sigmoid colon. In some embodiments, the oral drug dosage form is configured to release the drug at, near, or upstream of the rectum.

Within the desired location of the colon for drug release, the oral drug dosage forms described herein may be configured to release a drug according to any release profile. In some embodiments, the oral drug dosage form is configured to release a drug according to a controlled-release profile. In some embodiments, the controlled-release profile is an immediate-release profile. For example, as described herein, the drug component releases a drug contained therein in about 1 hour or less, such as about any of 50 minutes or less, 40 minutes or less, 30 minutes or less, 25 minutes or less, 20 minutes or less, 15 minutes or less, 10 minutes or less, 9 minutes or less, 8 minutes or less, 7 minutes or less, 6 minutes or less, 5 minutes or less, 4 minutes or less, 3 minutes or less, 2 minutes or less, or 1 minute or less. In some embodiments, the controlled-release profile is a sustained-release profile. For example, as described herein, the drug component releases a drug for at least about 1 hour, such as at least about any of 1.5 hours, 2 hours, 2.5 hours, 3 hours, 3.5 hours, 4 hours, 4 0.5 hours, 5 hours, 5.5 hours, 6 hours, 6.5 hours, or 7 hours. In some embodiments, the drug component of an oral drug dosage form is configured to release a drug from the oral drug dosage form for at least about 1 hour, such as at least about any of 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, or 8 hours.

In some embodiments, the oral drug dosage form is configured to prov ide a drug release profile comprising a zero-order release profile, first-order release profile, delayed release profile, pulsed release profile, iterative pulsed release profile, immediate release profile, or sustained release profile, or a combination thereof.

As described herein, in some embodiments, the oral drug dosage form may comprise another drug (including two or more, three or more, four or more, or five or more). In such embodiments, the other drug (s) may be released according to any desired drug release profile. In some embodiments, the desired drug release profile of another drug comprises a zero-order release profile, a first-order release pro file, a delayed release profile, a pulsed release profile, an iterative pulsed release profile, an immediate release profile, a sustained release profile, or a combination thereof.

In some embodiments, the release of the drug is based, at least in part, on an in vitro dissolution study, such as described in U.S. Pat. No. 10,350,822, which is hereby incorporated herein by references in its entirety. In some embodiments, the release of the drug is based, at least in part, on an in vivo dissolution study, such as described in in U.S. Pat. Publication No. 2021/0196638 A, which is hereby incorporated herein by references in its entirety.

### III. Commercial Batch

In some aspects, provided herein is a commercial batch of at least about 100 oral drug dosage forms described herein. In some embodiments, the commercial batch comprises at least about any of 250, 500, 750, 1,000, 2,500, 5,000, 7,500, 10,000, 20,000, 30,000, 40,000, 50,000, 60,000, 70,000, 80,000, 90,000 or 100,000 oral drug dosage forms described herein.

In some embodiments, the commercial batch has a standard deviation of about 0.1 or less, such as 0.05 or less, for one or more of the following: an amount of a drug and/or an agent, such as a bacterial degradation moiety, in the oral drug dosage form; a weight of the oral drug dosage form; a largest crossing dimension of the oral drug dosage form; a crossing dimension perpendicular to the largest crossing dimension of the oral drug dosage form; and a layer thickness.

### IV. Methods of Making and Designing Oral Drug Dosage Forms Described Herein

In some aspects, provided herein are methods of making an oral drug dosage form described herein. In some embodiments, the method of making comprises a three-dimensional (3D) printing technique to form at least one of the components, or a portion thereof, of the oral drug dosage forms described herein. In some embodiments, 3D printing techniques enable accurate control of drug release can be achieved by fine-tuning different parameters, such as geometry, infill percentages or composition.

In some embodiments, provided is a method of three-dimensional (3D) printing of an oral drug dosage form, the method comprising dispensing materials according to a layer-by-layer model of the oral drug dosage form (such as based on the thickness of material deposited by a pass of a printer head) to print the oral drug dosage form, wherein each layer of the layer-by-layer model is printed by dispensing, as necessary, for a layer: (a) a shell material to form any portion of a shell in the layer; (b) a drug component material to form any portion of a drug component in the layer; (c) a pH-based enteric member material to form any portion of a pH-based enteric member in the layer; (d) an erodible delay member material to form any portion of an erodible delay member in the layer; (e) a bacterial degradation member material to form any portion of a bacterial degradation member in the layer; and (f) an excipient component material to form any portion of an excipient component in the layer.

In some embodiments, the oral drug dosage form comprises (a) a drug component comprising an erodible material admixed with a drug; and (b) a pH-based enteric member (not admixed with the drug) comprising an erodible material configured to erode at or above a pre-determined pH value, the method of 3D printing comprising dispensing materials according to a layer-by-layer model of the oral drug dosage form (such as based on the thickness of material deposited by a pass of a printer head) to print the oral drug dosage form, wherein each layer of the layer-by-layer model is printed by dispensing, as necessary, for a layer: (a) a drug component material to form any portion of the drug component in the layer; and (b) the pH-based enteric member material to form any portion of a pH-based enteric member in the layer. In some embodiments, the method further comprises generating the layer-by-layer model of the oral drug dosage form.

In some embodiments, the oral drug dosage form comprises (a) a drug component comprising an erodible material admixed with a drug; (b) a pH-based enteric member (not admixed with the drug) comprising an erodible material configured to erode at or above a pre-determined pH value; and (c) an erodible delay member comprising an erodible material not admixed with the drug, the method of 3D printing comprising dispensing materials according to a layer-by-layer model of the oral drug dosage form (such as based on the thick ness of material deposited by a pass of a printer head) to print the oral drug dosage form, wherein each layer of the layer-by-layer model is printed by dispensing, as necessary, for a layer: (a) a drug component material to form any portion of the drug component in the layer; (b) a pH-based enteric member material to form any portion of the pH-based enteric member in the layer; and (c) an erodible delay member material to form any portion of the erodible delay member in the layer. In some embodiments, the method further comprises generating the layer-by-layer model of the oral drug dosage form.

In some embodiments, the oral drug dosage form comprises (a) a drug component comprising an erodible material admixed with a drug; (b) a pH-based enteric member (not admixed with the drug) comprising an erodible material configured to erode at or above a pre-determined pH value; and (c) a bacterial degradation member comprising a bacterial degradation moiety, the method of 3D printing comprising dispensing materials according to a layer-by-layer model of the oral drug dosage form (such as based on the thickness of material deposited by a pass of a printer head) to print the oral drug dosage form, wherein each layer of the layer-by-layer model is printed by dispensing, as necessary, for a layer: (a) a drug component material to form any portion of the drug component in the layer; (b) a pH-based enteric member material to form any portion of the pH-based enteric member in the layer; and (c) a bacterial degradation member material to form any portion of the bacterial degradation member in the layer. In some embodiments, the method further comprises generating the layer-by-layer model of the oral drug dosage form.

In some embodiments, the oral drug dosage form comprises (a) a drug component comprising an erodible material admixed with a drug; (b) a pH-based enteric member (not admixed with the drug) comprising an erodible material configured to erode at or above a pre-determined pH value; (c) an erodible delay member comprising an erodible material not admixed with the drug; and (d) a bacterial degradation member comprising a bacterial degradation moiety, the method of 3D printing comprising dispensing materials according to a layer-by-layer model of the oral drug dosage form (such as based on the thickness of material deposited by a pass of a printer head) to print the oral drug dosage form, wherein each layer of the layer-by-layer model is printed by dispensing, as necessary, for a layer: (a) a drug component material to form any portion of the drug component in the layer; (b) a pH-based enteric member material to form any portion of the pH-based enteric member in the layer; (c) an erodible delay member material to form any portion of the erodible delay member in the layer; and a bacterial degradation member material to form any portion of the bacterial degradation member in the layer. In some embodiments, the method further comprises generating the layer-by-layer model of the oral drug dosage form.

In some embodiments, the oral drug dosage form comprises (a) a drug component comprising an erodible material admixed with a drug; (b) a pH-based enteric member (not admixed with the drug) comprising an erodible material configured to erode at or above a pre-determined pH value; and (c) an excipient component comprising an excipient, the method of 3D printing comprising dispensing materials according to a layer-by-layer model of the oral drug dosage form (such as based on the thickness of material deposited by a pass of a printer head) to print the oral drug dosage form, wherein each layer of the layer-by-layer model is printed by dispensing, as necessary, for a layer: (a) a drug component material to form any portion of the drug component in the layer; (b) a pH-based enteric member material to form any portion of the pH-based enteric member in the layer; and (c) an excipient component material to form any portion of the excipient component in the layer. In some embodiments, the method further comprises generating the layer-by-layer model of the oral drug dosage form.

In some embodiments, the oral drug dosage form comprises (a) a drug component comprising an erodible material admixed with a drug; (b) a pH-based enteric member (not admixed with the drug) comprising an erodible material configured to erode at or above a pre-determined pH value; (c) an erodible delay member comprising an erodible material not admixed with the drug; and (d) an excipient component comprising an excipient, the method of 3D printing comprising dispensing materials according to a layer-by-layer model of the oral drug dosage form (such as based on the thickness of material deposited by a pass of a printer head) to print the oral drug dosage form, wherein each layer of the layer-by-layer model is printed by dispensing, as necessary, for a layer: (a) a drug component material to form any portion of the drug component in the layer; (b) a pH-based enteric member material to form any portion of the pH-based enteric member in the layer; (c) an erodible delay member mate rial to form any portion of the erodible delay member in the layer; and (d) an excipient component material to form any portion of the excipient component in the layer. In some embodiments, the method further comprises generating the layer-by-layer model of the oral drug dosage form.

In some embodiments, the oral drug dosage form comprises (a) a drug component comprising an erodible material admixed with a drug; (b) a pH-based enteric member (not admixed with the drug) comprising an erodible material configured to erode at or above a pre-determined pH value; (c) an erodible delay member comprising an erodible material not admixed with the drug; (d) a bacterial degradation member comprising a bacterial degradation moiety; and (e) an excipient component comprising an excipient, the method of 3D printing comprising dispensing materials according to a layer-by-layer model of the oral drug dosage form (such as based on the thickness of material deposited by a pass of a printer head) to print the oral drug dosage form, wherein each layer of the layer-by-layer model is printed by dispensing, as necessary, for a layer: (a) a drug component material to form any portion of the drug component in the layer; (b) a pH-based enteric member material to form any portion of the pH-based enteric member in the layer; (c) an erodible delay member material to form any portion of the erodible delay member in the layer; (d) a bacterial degradation member material to form any portion of the bacterial degradation member in the layer; and (e) an excipient component material to form any portion of the excipient component in the layer. In some embodiments, the method further comprises generating the layer-by-layer model of the oral drug dosage form.

For any of the embodiments above, the oral drug dosage form may further comprise a shell not admixed with the drug. For embodiments wherein the oral drug dosage form comprises a shell, the method further comprises dispensing materials according to a layer-by-layer model of the oral drug dosage form (such as based on the thickness of material deposited by a pass of a printer head) to print the oral drug dosage form, wherein each layer of the layer-by-layer model is printed by dispensing, as necessary, for a layer, a shell material to form any portion of the shell in the layer.

As used herein, “printing,” “three-dimensional printing,” “3D printing,” “additive manufacturing,” or equivalents thereof, refers to a process that pro duces three-dimensional objects, such as oral drug dosage forms, layer-by-layer using digital designs. The basic process of three-dimensional printing has been described in U.S. Pat. Nos. 5,204,055; 5,260,009; 5,340,656; 5,387,380; 5,503,785; and 5,633,021. Additional U.S. patents and patent applications that related to three-dimensional printing include: U.S. Pat. Nos. 5,490,962; 5,518,690; 5,869,170; 6,530,958; 6,280,771; 6,514,518; 6,471,992; 8,828,411; U.S. Publication Nos. 2002/0015728; 2002/0106412; 2003/0143268; 2003/0198677; 2004/000 5360. The contents of the above U.S. patents and patent applications are hereby incorporated herein by reference in their entirety. In some embodiments, an additive manufacturing technique is used to produce the oral drug dosage forms described herein. In some embodiments, a layer-by-layer technique is used to produce the oral drug dosage forms described herein. Because 3D printing may handle a range of pharmaceutical materials and control both composition and architecture locally, 3D printing is well suited to the fabrication of oral drug dosage forms with complex geometry and compositions in accordance with the present invention.

In some embodiments, layer, when used in reference to, e.g., a drug component layer or a delay component layer (e.g., a pH-based enteric member layer and/or an erodible delay member layer), refers to the configuration of a component of the oral drug dosage form and may comprise a plurality of printed layers of the same material. In some embodiments, the layer has a pre-determined fill density, such a three-dimensional printed fill density. In some embodiments, the layer, such as the drug component layer, comprises a plurality of printed lay ers of between about 5 printed layers to about 2500 printed layers, such as between any of about 10 printed layers to about 2500 printed layers, about 25 printed layers to about 100 printed layers, about 50 printed layers to about 200 printed layers, about 100 printed layers to about 200 printed layers, about 150 printed layers to about 2 50 printed layers, about 200 printed layers to about 250 printed layers, about 500 printed layers to about 1000 printed layers, or about 2000 printed layers to about 2400 printed layers. In some embodiments, the thickness of a printed layer is no more than about 5 mm, such as no more than about any of 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, or 0.01 mm. In some embodiments, the thickness of a printed layer is about any of 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, or 0.01 mm.

Different 3D printing methods have been developed for manufacturing in terms of raw materials, equipment, and solidification. These 3D printing methods include binder deposition (see Gibson et al., Additive Manufacturing Technologies: 3D Printing, Rapid Prototyping, and Direct Digital Manufacturing., 2 ed. Springer, New York, 2015; Katstra et al., Oral dosage forms fabricated by three dimensional printing, J Control Release, 66, 2000; Katstra et al., Fabrication of complex oral delivery forms by three dimensional printing, Dissertation in Materials Science and Engineering, Massachusetts Institute of Technology, 2001; Lipson et al., Fabricated: The New World of 3D printing, John Wiley & Sons, Inc., 2013; Jonathan, Karim 3D printing in pharmaceutics: a new tool for designing customized drug delivery systems, Int J Pharm, 499, 2016), material jetting (see Jonathan, Karim, 3D printing in pharmaceutics: a new tool for designing customized drug delivery systems, Int J Pharm, 49 9, 2016), extrusion (see Gibson et al., Additive Manufacturing Technologies: 3D Printing, Rapid Prototyping, and Direct Digital Manufacturing. 2 ed. Springer, New York, 2015), and photopolymerization (see Melchels et al., A review on stereolithography and its application in biomedical engineering. Biomaterials, 31, 2010).

In some embodiments, the oral drug dosage forms described herein are 3D printed using an extrusion method. In some embodiments, the method of 3D printing comprises using a double screw extrusion method. In an extrusion process, material is extruded from robotically-actuated printing heads through printing nozzles. Unlike binder deposition, which requires a powder bed, extrusion methods can print on any substrate. A variety of materials can be extruded for three-dimensional printing, including thermoplastic materials disclosed herein, pastes and colloidal suspensions, silicones, and other semisolids. One extrusion printing method is melt extrusion deposition (MED), which used extruded material from a printing head to print layers of material to form the components of the oral drug dosage form. Another common type of extrusion printing is fused deposition modeling, which uses solid polymeric filaments for printing. In fused deposition modeling, a gear system drives the filament into a heated nozzle assembly for extrusion (see Gibson et al., Additive Manufacturing Technologies: 3D Printing, Rapid Prototyping, and Direct Digital Manufacturing, 2 ed. Springer, New York, 2015).

In some embodiments, the 3D printing is carried out by semisolid extrusion, such as semisolid extrusion of a material comprising a biological drug, e.g., a polypeptide or a nucleic acid. In some embodiments, the 3D printing is carried out by MED and semisolid extrusion, e.g., semisolid extrusion to produce a drug component admixed with a biological drug and MED to produce other components. In some embodiments, the 3D printing is carried out by MED to produce a delay component or shell and then the 3D printing is carried out by semisolid extrusion to produce a drug component admixed with abiological drug at lower temperatures than MED. In some embodiments, the drug in drug component can maintain stable by semisolid extrusion.

In some embodiments, the 3D printing is carried out by melt extrusion deposition (MED). In some embodiments, the melt extrusion deposition technique comprises preparing a material to be dispensed, such as preparing a pow der in a hot melt extruder, and then feeding the material into a MED printing head. The MED printing head then dispenses the material to form the oral drug dosage form in an additive manner (layer-by-layer deposition). In some embodiments, each material of the oral drug dosage form, such as the sustained-release drug component, the delay member, and the shell, is dispensed from a different MED printing head. In some embodiments, the MED printing head dispenses the material according to instructions complied in one or more G code files. Exemplary MED techniques are disclosed in, e.g., WO2018/210183, WO2019/137333, WO2018137686, and U.S. Pat. No. 10,201,503, each of which is incorporated herein by reference in its entirety.

In some embodiments, the melt extrusion deposition 3D printing technique comprises: (a) preparing each component material by melting and extrusion of the component material; and (b) printing an oral drug dosage form using a layer-by-layer technique. In some embodiments, the melt extrusion deposition 3D printing technique further comprises preparing a printer head for printing. In some embodiments, preparing the drug component material comprises melting and extrusion of the drug component material. In some embodiments, preparing the drug component material comprises mixing the ingredients of the drug component material, such as any thermoformable components and a drug. In some embodiments, preparing the drug component material comprises weighing each ingredient thereof, mixing the ingredients, and melting and extrusion of the drug component material. In some embodiments, preparing the shell material comprises melting and extrusion of the shell material. In some embodiments, preparing the shell material comprises mixing the ingredients of the shell material, such as any thermoformable components thereof. In some embodiments, preparing the shell material comprises weighing each ingredient thereof, mixing the ingredients, and melting and extrusion of the shell material. In some embodiments, preparing the pH-based enteric member material comprises melting and extrusion of the pH-based enteric member material. In some embodiments, preparing the pH-based enteric member material comprises mixing the ingredients of the pH-based enteric member material, such as any thermoformable components thereof. In some embodiments, preparing the pH-based enteric member material comprises weighing each ingredient thereof, mixing the ingredients, and melting and extrusion of the pH-based enteric member material. In some embodiments, preparing the erodible delay member material comprises melting and extrusion of the erodible delay member material. In some embodiments, preparing the erodible delay material comprises mixing the ingredients of the erodible delay material, such as any thermoformable components thereof. In some embodiments, preparing the erodible delay material comprises weighing each ingredient thereof, mixing the ingredients, and melting and extrusion of the erodible delay material. In some embodiments, preparing the bacterial degradation member material comprises melting and extrusion of the bacterial degradation member material. In some embodiments, preparing the bacterial degradation member material comprises mixing the ingredients of bacterial degradation member material, such as any thermoformable components and a bacterial degradation moiety. In some embodiments, preparing the bacterial degradation member material comprises weighing each ingredient thereof, mixing the ingredients, and melting and extrusion of the bacterial degradation member material. In some embodiments, preparing the excipient component material comprises melting and extrusion of the excipient component material. In some embodiments, preparing the excipient component material comprises mixing the ingredients of excipient component material, such as any thermoformable components and a excipient moiety. In some embodiments, preparing the excipient component material comprises weighing each ingredient thereof, mixing the ingredients, and melting and extrusion of the excipient component material.

In some embodiments, preparing the printer head for printing comprises loading the formed component material, such as a drug component material, into the printer head. In some embodiments, preparing the printer head for printing comprises setting the printer head temperature. In some embodiments, preparing the printer head for printing comprises setting and applying the feeding pressure to the printer head. In some embodiments, applying the feeding pressure is completed after the printer head temperature is at a pre-determined level. In some embodiments, printing of the oral drug dosage form is performed layer-by-layer (e.g., additive manufacturing). In some embodiments, the method comprises using a separate printer head for each component material (e.g., a first printer head to dispense a drug component material, a second printer head to dispense a shell material, a third printer head to dispense a pH-based enteric member material, and a fourth printer head to dispense an erodible delay member material). In some embodiments, the method of making is designed and performed based on a desired total number of an oral drug dosage form to be produced in a production run. For example, in some embodiments, a smaller production run (such as less than 1,000 oral drug dosage forms for product development or a clinical trial) is desired and the method of making comprises preparing each component material (such as by weighing the ingredients of a component material and forming the component material by mixing the ingredients and hot melt extrusion), and then printing each oral drug dosage form layer-by-layer (e.g., additive manufacturing). In some embodiments, a larger production run (such as a commercial mass production run of more than 1,000 of an oral drug dosage form) is desired and the method of making comprises preparing each component material (such as by weighing the ingredients of a component material and forming the component material by mixing the ingredients and hot melt extrusion, wherein the hot melt extrusion is performed using a twin-screw extruder), and then printing each oral drug dosage form layer-by-layer (e.g., additive manufacturing). In some embodiments, the component material is formed using, at least in part, a twin-screw extruder. In some embodiments, the larger production run comprises distributing the component material from the twin-screw extruder to each printer head via a flow distribution module. In some embodiments, the larger production run is performed via the cooperation of multiple modules. For example, in some embodiments, the method of making comprises use of a system comprising a material supply module for receiving a set of component materials for printing; a flow distribution module comprising a flow distribution plate, wherein the material supply module is configured to transport a single flow corresponding to the set of component materials for printing to the flow distribution plate; wherein the flow distribution plate comprises a plurality of channels for dividing the single flow into a plurality of flows; a plurality of nozzles; and one or more controllers for controlling the plurality of nozzles to dispense the plurality of flows based on a plurality of nozzle-specific parameters. In some embodiments, the system further comprises a printing platform configured to receive the dispensed plurality of flows, wherein the printing platform is configured to move to form a batch of the pharmaceutical product. In some embodiments, the system comprises a plurality of printing platforms.

In some embodiments, the 3D printing is carried out by fused deposition modeling (FDM). In some embodiments, the three-dimensional printing is carried out by melt extrusion deposition or hot melt extrusion coupled with a 3D printing technique, such as FDM. In some embodiments, the 3D printing is carried out by non-filament FDM. In some embodiments, the 3D printing is carried out by inkjet printing. In some embodiments, the 3D printing is carried out by selective laser sintering (SLS). In some embodiments, the 3D printing is carried out by stereolithography (SLA or SL). In some embodiments, the 3D printing is carried out by PolyJet, Multi-Jet Printing System (MJP), Perfactory, Solid Object Ultraviolet-Laser Printer, Bioplotter, 3D Bioprinting, Rapid Freeze Prototyping, Benchtop System, Selective Deposition Lamination (SDL), Laminated Object Manufacturing (LOM), Ultrasonic Consolidation, ColorJet Printing (CJP), EOSINT Systems, Laser Engineered Net Shaping (LENS) and Aerosol Jet System, Electron Beam Melting (EBM), Laser CUSING®, Selective Laser Melting (SLM), Phenix PX™ Series, Microsintering, Digital Part Materialization (DPM), or VX System.

In some embodiments, the 3D printing methods described herein comprise a continuous feed method. In some embodiments, the 3D printing methods described herein comprise a batch feed method.

The method instructions for 3D printing a drug dosage form disclosed herein may be generated a variety of ways, including direct coding, derivation from a solid CAD model, or other means specific to the 3D printing machine's computer interface and application software. These instructions may include information on the number and spatial placement of droplets, and on general 3D print parameters such as the drop spacing in each linear dimension (X, Y, Z), and volume or mass of fluid per droplet. For a given set of materials, these parameters may be adjusted in order to refine the quality of structure created. The overall resolution of the structure created is a function of the powder particle size, the fluid droplet size, the print parameters, and the material properties.

In some embodiments, one or more components of the oral drug dosage form are created separately, such as printed separately, and later assembled to form the oral drug dosage form. In some embodiments, all components of the oral drug dosage form are created in a single method, such as printed in a single method, without requiring later assembly. For example, in some embodiments, the oral drug dosage form is produced using a layer-by-layer 3D printing technique.

The oral drug dosage forms and components thereof described in the present application can be printed on a commercial scale. For example, in some embodiments, the methods disclosed herein may be used to 3D print 10,000 to 100,000 units of an oral drug dosage form per hour. In some embodiments, the methods disclosed herein may be used to 3D print 10,000 to 100,000 oral drug dosage forms per hour. In some embodiments, the methods disclosed herein may be used to 3D print 10,000 to 100,000 units of a dosage unit per hour. In some embodiments, the methods disclosed herein may be used to 3D print 10,000 to 100,000 dosage units per hour.

The 3D printing methods described herein encompass printing the mate rials in any order that will allow for production of the oral drug dosage form, or components thereof.

In some embodiments, the method for 3D printing comprises designing the oral drug dosage form, or component thereof, in whole or in part, on a computer system. In some embodiments, the method comprises inputting parameters of the desired locational drug release profile and/or the oral drug dosage form into the computer system. In some embodiments, the method comprises pro viding one or more parameters to be printed, e.g., layer surface area, thickness, drug mass fraction, erosion rate. In some embodiments, the method comprises providing the desired drug release profile. In some embodiments, the methods comprise creating a virtual image of the item to be printed. In some embodiments, the method comprises creating a computer model that contains the pre-determined parameters. In some embodiments, the method comprises feeding the pre-determined parameters to a 3D printer and printing the item according to such pre-determined parameters. In some embodiments, the method comprises creating a 3D drawing of the item to be printed based on the pre-determined parameters, wherein the 3D drawing is created on a computer system. In some embodiments, the method comprises converting, such as slicing, a 3D drawing into 3D printing code, e.g., G code. In some embodiments, the method comprises using the computer system to execute 3D printing code, thereby printing according to the methods described herein.

In some aspects, the methods provided herein comprise one or more packaging steps. In some embodiments, the packaging step comprises packaging each individual dosage form into a discrete container, such as laminated film and pouches for pharmaceutical packaging. In some embodiments, the packaging step comprises packaging a number of packaged dosage forms into a carton.

In some embodiments, the method further comprises one or more in-process quality control steps. For example, in some embodiments, after making the dosage form, the in-process quality control step comprises one or more of assessing a dosage form for appearance or a feature thereof, assessing the dosage form for weight, and assessing the dosage form for dimensions. In some embodiments, to pass the in-process quality control step, the evaluated characteristic must be within a pre-determined thresh old. In some embodiments, after each dosage for is packaged, the in-process quality control step comprises assess the seal of each dosage form, e.g., for tightness, and/or filing quantity. In some embodiments, after packaging in the carton, the in-process quality control step comprises confirming the filing quantity of the carton.

In some embodiments, the methods of designing provided herein comprising using one or more tracking stripes such that external imagine techniques, e.g., x-ray imaging, can be used to assess the status (such as not yet undergoing erosion/release from the oral drug dosage form, undergoing erosion/release from the oral drug dosage form, or completion of erosion/release from the oral drug dosage form) of one or more components of an oral drug dosage form taught herein relative to the location of the oral drug dosage form in the individual to which it was administered. For example, such tracking features then enable one to confirm that one or more components on an oral drug dosage form are behaving as desired, such as release of a drug from a drug component is occurring at a desired location in the gastrointestinal tract of an individual. This further enables methods comprising adjusting the design of an oral drug dosage form to provide the desired results. For instances, if it is found that a drug component is eroding and releasing drug in a location earlier than desired (e.g., upstream of the desired location in the gastrointestinal tract), then one or more aspects of the delay component can be adjusted, such as by changing a composition or thickness of a material thereof.

In some embodiments, provided is a method of designing an oral drug dosage form configured to release a drug at a desired gastrointestinal location in an individual, wherein the oral drug dosage form comprises: a drug component comprising: a first erodible material admixed with a drug, and a first erodible tracking stripe; and a shell comprising a second tracking stripe; a delay component not admixed with the drug, the delay component comprising: a pH-based enteric member configured to erode at or above a pre-determined pH value, wherein the pH based enteric member comprises a third erodible tracking stripe; and an erodible delay member comprising a second erodible material, wherein the erodible delay member comprises a fourth erodible tracking stripe, wherein the pH-based enteric member, alone or in combination with the shell, prevents erosion of the erodible delay member, and wherein the erodible delay member, alone or in combination with the shell, prevents erosion of the drug component, the method comprising: (a) administering the oral drug dosage form to an individual; (b) imaging the individual over a time course to obtain a location of the oral drug dosage form and a status of the first erodible tracking stripe, the second erodible tracking stripe, the third erodible tracking stripe, and the fourth tracking stripe; and (c) adjusting the drug component and/or delay component and/or shell, or portion thereof, based on the location and the status of the first erodible tracking stripe, the second erodible tracking stripe, the third erodible tracking stripe, and the fourth track stripe to design the oral drug dosage form configured to release a drug at a desired gastrointestinal location in an individual.

In some embodiments, the adjusting the delay component comprises adjusting the pH-based enteric member by changing one or more of the composition, sur face area exposed to bodily fluid following administration, or thickness of the pH-based enteric member. In some embodiments, the adjusting the delay component comprises adjusting the erodible delay member by changing one or more of the composition, surface area exposed to bodily fluid following administration, or thickness of the erodible delay member.

In some embodiments, provided herein is a method of designing an oral drug dosage form configured to release a drug at a desired gastrointestinal location in an individual, wherein the oral drug dosage form comprises: a drug component comprising: a first erodible material admixed with a drug, and a first erodible tracking stripe; and a shell; a delay component not admixed with the drug, the delay component comprising: a pH-based enteric member configured to erode at or above a pre-determined pH value; and an erodible delay member comprising a second erodible material, wherein the pH-based enteric member, alone or in combination with the shell, prevents erosion of the erodible delay member, and wherein the erodible delay member, alone or in combination with the shell, prevents erosion of the drug component, the method comprising: (a) administering the oral drug dosage form to an individual; (b) imaging the individual over a time course to obtain a location of the oral drug dosage form and a status of the first erodible tracking stripe; and (c) adjusting the drug component and/or delay component and/or shell, or portion thereof, based on the location and the status of the first erodible tracking stripe, to design the oral drug dosage form configured to release a drug at a desired gastrointestinal location in an individual.

In some embodiments, the first erodible material admixed with the drug and the first erodible tracking stripe are not in direct contact with one another. In some embodiments, the shell comprises a second tracking stripe, and wherein the second tracking stripe is configured to be imaged by the imaging such as to obtain a status of the second tracking stripe. In some embodiments, the adjusting is based on the location and the status of the first erodible tracking stripe and/or the second tracking stripe. In some embodiments, the pH-based enteric member comprises a third tracking stripe, and wherein the third tracking stipe is configured to be imaged by the imaging such as to obtain a status of the third erodible tracking stripe. In some embodiments, the adjusting is based on the location and the status of the first erodible tracking stripe and/or the second tracking stripe and/or the third erodible tracking stripe. In some embodiments, the erodible delay member comprises a fourth tracking stripe, and wherein the fourth tracking stripe is configured to be imaged by the imaging such as to obtain a status of the fourth erodible tracking stripe. In some embodiments, the adjusting is based on the location and the status of the first erodible tracking stripe and/or the second tracking stripe and/or the third erodible tracking stripe and/or the fourth erodible tracking stripe.

In some embodiments, the imaging provides a location of the oral drug dosage form when the first erodible tracking stripe begins to erode. In some embodiments, the imaging provides a location of the oral drug dosage form when the first erodible tracking stripe has completely eroded. In some embodiments, the adjusting comprises adjusting the delay component, or portion thereof, to increase the delay of release of the drug from the oral drug dosage form based on the location and the status of the first erodible tracking stripe showing earlier than desired release. In some embodiments, the adjusting comprises adjusting the delay component, or portion thereof, to decrease the delay of release of the drug from the oral drug dosage form based on the location and the status of the first erodible tracking stripe showing later than desired release.

In some embodiments, the first tracking stripe comprises barium sulfate. In some embodiments, the imagining comprises x-ray imaging. In some embodiments, the method further comprises obtaining one or more pharmacokinetic (PK) parameters associated with the drug following the administration of the oral drug dosage form to an individual. In some embodiments, the method further comprises identifying a relationship between the one or more PK parameters and the locational and status information of the first tracking stripe. In some embodiments, the method further comprises adjusting the drug component and/or delay component, or portion thereof, based on the relationship between the one or more PK parameters and the locational and status information of the first tracking stripe. In some embodiments, the imaging does not involve a magnetic-based technique or an invasive imaging technique.

In some embodiments, provided herein is a method of designing an oral drug dosage form configured to release a drug at a desired gastrointestinal location in an individual, wherein the oral drug dosage form comprises: a drug component comprising a first erodible material admixed with a drug; a shell; and a delay component not admixed with the drug, the delay component comprising: a pH-based enteric member configured to erode at or above a pre-determined pH value; and an erodible delay member comprising a second erodible material, wherein the pH-based enteric member prevents erosion of the erodible delay member, and wherein the erodible delay member prevents erosion of the drug component, wherein the oral drug dosage form comprises at least one tracking stripe positioned in a component thereof, the method comprising: (a) administering the oral drug dosage form to an individual; (b) imaging the individual over a time course to obtain a location of the oral drug dosage form and a status of the at least one tracking stripe; and (c) adjusting the drug component and/or delay component and/or shell, or portion thereof, based on the location and the status of the at least one tracking stripe, to design the oral drug dosage form configured to release a drug at a desired gastrointestinal location in an individual.

In some embodiments, one of the tracking stripes is located in, or near, the drug component. In some embodiments, the tracking stripes is located in the shell. In some embodiments, one of the tracking stripes is located in the pH-based enteric member. In some embodiments, one of the tracking stripes is located in the erodible delay member. In some embodiments, at least one of the at least one tracking stripes is erodible.

In some embodiments, provided herein is a method of designing an oral drug dosage form configured to release a drug at a desired gastrointestinal location in an individual, wherein the oral drug dosage form comprises: a drug component comprising a first erodible material admixed with a drug; and a delay component not admixed with the drug, the delay component comprising: a pH-based enteric member configured to erode at or above a pre-determined pH value; and an erodible delay member comprising a second erodible material, wherein the pH-based enteric member prevents erosion of the erodible delay member, and wherein the erodible delay member prevents erosion of the drug component, wherein the oral drug dosage form comprises at least one tracking stripe positioned in a component thereof, the method comprising: (a) administering the oral drug dosage form to an individual; (b) imaging the individual over a time course to obtain a location of the oral drug dosage form and a status of the at least one tracking stripe; and (c) adjusting the drug component and/or delay component, or portion thereof, based on the location and the status of the at least one tracking stripe, to design the oral drug dosage form configured to release a drug at a desired gastrointestinal location in an individual.

In some embodiments, one of the tracking stripes is located in, or near, the drug component. In some embodiments, one of the tracking stripes is located in the pH-based enteric member. In some embodiments, one of the tracking stripes is located in the erodible delay member. In some embodiments, at least one of the at least one tracking stripes is erodible.

In some embodiments, provided is a method of designing and/or adjusting an oral drug dosage form described herein configured to release a drug at a desired location in the colon of an individual. In some embodiments, the adjusting comprises adjusting the delay component, or portion thereof, to increase the delay of release of the drug from the oral drug dosage form. As described herein, “adjusting” can re fer to making a change in a parameter that, e.g., influences the rate at which a component erodes from an oral drug dosage form. For example, in some embodiment, “adjusting” can refer to making a change in one or more of thickness, drug mass fraction, density, composition, surface area exposed to a bodily fluid, or erosion rate of a material. In some embodiments, the adjusting is based on data obtained from studying an oral drug dosage form described herein, such as in vitro and/or in vivo studies, e.g., cumulative dissolution profiles or PK studies. Techniques for adjusting features of an oral drug dosage form to increase or decrease the delay of release of the drug therefrom are known in the art, and include, e.g., adjustments to thickness, surface area, and a material property. Adjustment techniques are described in U.S. Pat. No. 10,350,822, which is hereby incorporated herein by references in its entirety.

In some embodiments, the design method further comprise obtaining, such as measuring, one or more pharmacokinetic (PK) parameters associated with the drug following the administration of the oral drug dosage form to an in dividual. PK parameters are well known in the art and include, e.g., T_{det}, Tₘₐₓ, Cₘₐₓ, and AUC. Design methods involving PK parameters are described in U.S. Pat. Publication No. 2021/0196638 A, which is hereby incorporated herein by references in its entirety.

V. Methods of Treating and/or Delivery

In some aspects, provided herein are methods of treating a condition comprising administering (orally) an oral drug dosage form described herein to an in dividual. In some aspects, provided herein are methods of providing local delivery of a drug to the colon of an individual, the method comprising administering (orally) to the individual an oral drug dosage form described herein, e.g., to treat an individual is suffering from a gastrointestinal disorder. In some aspects, provided herein are methods of providing systemic delivery of a drug to an individual, the method comprising administering (orally) to the individual an oral drug dosage form described herein, e.g., to delivery a biological agent such as a peptide or nucleic acid.

The oral drug dosage forms described herein may comprise a diverse array of agents, and thus methods of use there may be useful for treating a wide array of conditions. In some embodiments, the condition is selected from the group consisting of a gastrointestinal disorder, central nervous system (CNS) disorder, cardiovascular disease, hypertension, atherosclerosis, angina, arterial obstruction, peripheral arterial disease, myocardial pathology, arrhythmia, acute myocardial infarction, angina, cardiomyopathy, congestive heart failure, coronary artery disease (CAD), carotid artery disease, endocarditis, hypercholesterolemia, hyperlipidemia, peripheral artery disease (PAD), genitourinary disorders; erectile dysfunction, urinary organ diseases benign prostatic hypertrophy (BPH), renal tubular acidosis, diabetic nephropathy, glomerulonephritis, glomerulosclerosis, urinary tract infection, faecal incontinence, ocular disease glaucoma, blepharitis, ocular hypertension, retinopathy, conjunctivitis, scleritis, retinitis, keratitis, corneal ulcer, iritis, chorioretinal inflammation, macular edema, xerophthalmia, pulmonary disease asthma, pulmonary hypertension, acute respiratory distress syndrome, COPD, emphysema, pneumonia, tuberculosis, bronchitis, acute bronchitis, bronchiectasis, bronchiolitis, bronchopulmonary dysplasia, byssinosis, coccidioidomycosis (Cocci), cystic fibrosis, influenza, lung cancer, mesothelioma, metabolic disease, hypercalciuria, hyperglycemia, hyperinsulinemic hypoglycemia, hyperinsulinism, hyperlysinuria, and hypoglycemia. In some embodiments, the gastrointestinal disorder is selected from the group consisting of irritable bowel disease (IBD), irritable bowel syndrome (IBS), constipation, diarrhea, infection, and carcinoma. In some embodiments, the IBD is associated with Crohn's disease or ulcerative colitis. In some embodiments, the carcinoma is colon cancer or colorectal cancer. In some embodiments, the CNS disorder is selected from the group consisting of neurogenic pain, stroke, dementia, Alzheimer's disease, Parkinson's disease, neuronal degeneration, meningitis, spinal cord injury, cerebral vasospasm, and amyotrophic lateral sclerosis.

In some embodiments, provided is a method of providing local delivery of a drug to the colon of an individual, the method comprising administering to the individual an oral drug dosage form described herein, wherein the individual is suffering from a gastrointestinal disorder. In some embodiments, the gastrointestinal dis order is selected from the group consisting of irritable bowel disease (IBD), irritable bowel syndrome (IBS), constipation, diarrhea, infection, and carcinoma. In some embodiments, the IBD is associated with Crohn's disease or ulcerative colitis. In some embodiments, the carcinoma is colon cancer or colorectal cancer. In some embodiments, oral drug dosage form comprises an anti-inflammatory agent, non-steroidal anti-inflammatory agent, or steroid. In some embodiments, the drug component comprises tofacitinib, e.g., tofacitinib citrate.

In some embodiments, provided is a method for treating ulcerative colitis, the method comprising administering to a human individual an oral drug dosage form described herein, wherein the oral drug dosage form comprises a JAK inhibitor. In some embodiments, the JAK inhibitor comprises tofacitinib. In some embodiments, the oral drug dosage form comprises about 11 mg or less of tofacitinib. In some embodiments, the oral drug dosage form comprises about 5.5 mg or less of tofacitinib. In some embodiments, the method comprises administering the oral drug dosage form once daily.

In some embodiments, provided is a method for treating ulcerative colitis, the method comprising administering to a human individual an oral drug dosage form described herein, wherein the drug component comprises a JAK inhibitor. In some embodiments, the JAK inhibitor comprises tofacitinib. In some embodiments, the oral drug dosage form comprises about 5.5 mg or less of tofacitinib. In some embodiments, the method comprises administering the oral drug dosage form once daily.

In some embodiments, provided is a method for treating ulcerative colitis, the method comprising administering to a human individual an oral drug dosage form described herein, wherein the drug component comprises tofacitinib 5.5 mg and the method comprises administering the oral drug dosage form once daily.

In some embodiments, provided is a method of providing systemic deli very of a drug to an individual, the method comprising administering (orally) to the individual an oral drug dosage form described herein, wherein the individual is suffering from a central nervous system (CNS) disorder, cardiovascular disease, hypertension, atherosclerosis, angina, arterial obstruction, peripheral arterial disease, myocardial pathology, arrhythmia, acute myocardial infarction, angina, cardiomyopathy, congestive heart failure, coronary artery disease (CAD), carotid artery disease, endocarditis, hypercholesterolemia, hyperlipidemia, peripheral artery disease (PAD), genitourinary disorders; erectile dysfunction, urinary organ diseases benign prostatic hypertrophy (BPH), renal tubular acidosis, diabetic nephropathy, glomerulonephritis, glomerulosclerosis, urinary tract infection, faecal incontinence, ocular disease glaucoma, blepharitis, ocular hypertension, retinopathy, conjunctivitis, scleritis, retinitis, keratitis, corneal ulcer, iritis, chorioretinal inflammation, macular edema, xerophthalmia, Pulmonary disease asthma, pulmonary hypertension, acute respiratory distress syndrome, COPD, emphysema, pneumonia, tuberculosis, bronchitis, acute bronchitis, bronchiectasis, bronchiolitis, bronchopulmonary dysplasia, byssinosis, coccidioidomycosis (Cocci), cystic fibrosis, influenza, lung cancer, mesothelioma, metabolic disease, hypercalciuria, hyperglycemia hyperinsulinemic hypoglycemia, hyperinsulinism, hyperlysinuria, or hypoglycemia. In some embodiments, the oral drug dosage form comprises a biological agent, such as a peptide, an antibody or a nucleic acid (e.g., RNA). In some embodiments, the RNA is selected from the group consisting of transfer RNA (tRNA), ribosomal RNA (rRNA) or messenger RNA (mRNA).

Those skilled in the art will recognize that several embodiments are possible within the scope and spirit of the disclosure of this application. The disclosure is illustrated further by the examples below, which are not to be construed as limiting the dis closure in scope or spirit to the specific procedures described therein.

### EXAMPLES

### Example 1

This example demonstrates the design, production, and testing of an oral drug dosage form described herein comprising tofacitinib citrate.

Release characteristics of four oral drug dosage form provided in Tables 1A-4B were first evaluated in vitro. As the oral drug dosage forms are designed to release drug from both sides of the oral drug dosage forms, during dissolution testing the oral drug dosage forms were adhered to the bottom of a steel rod within the testing vessel. The rod was fixed 1.5 cm from the center of the vessel, while the lower edge of the oral drug dosage form was adjusted to about 1 cm above the top of the paddle blade. The longest axis of the oral drug dosage form was oriented tangentially to the flow stream.

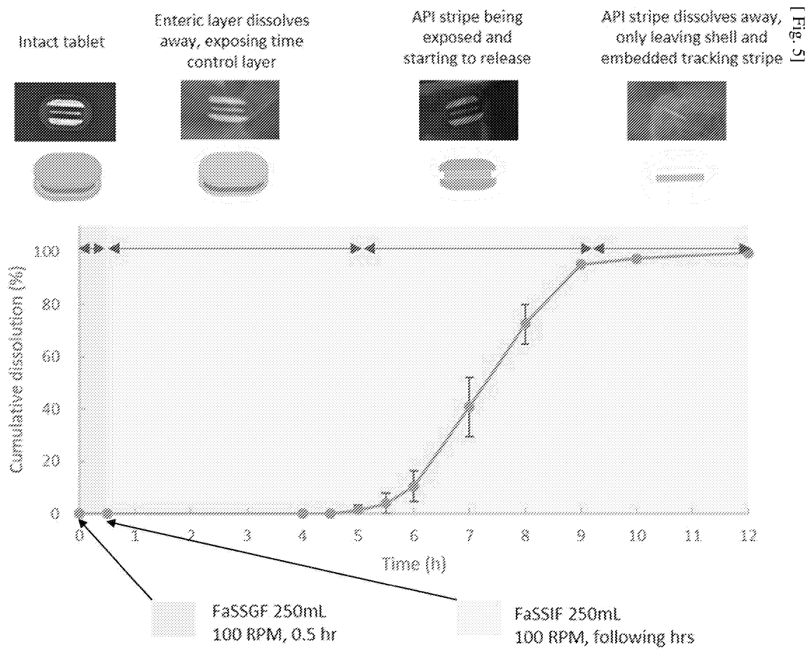

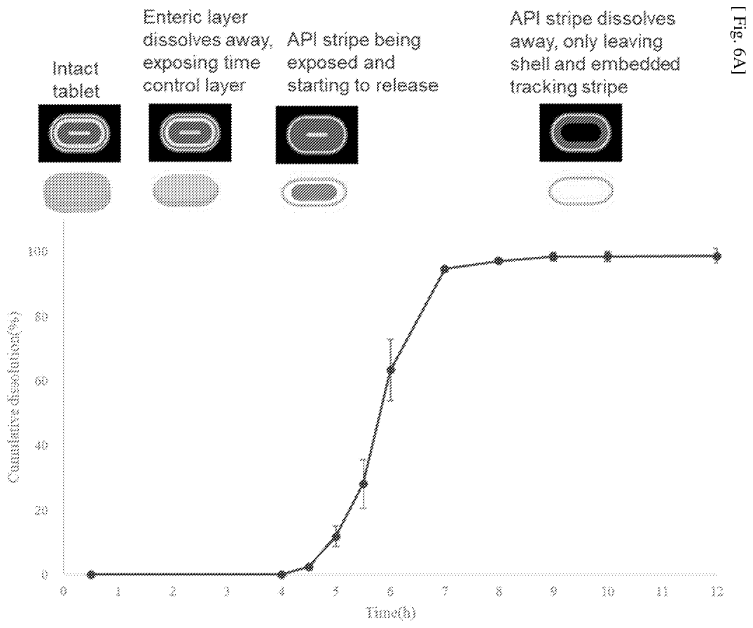

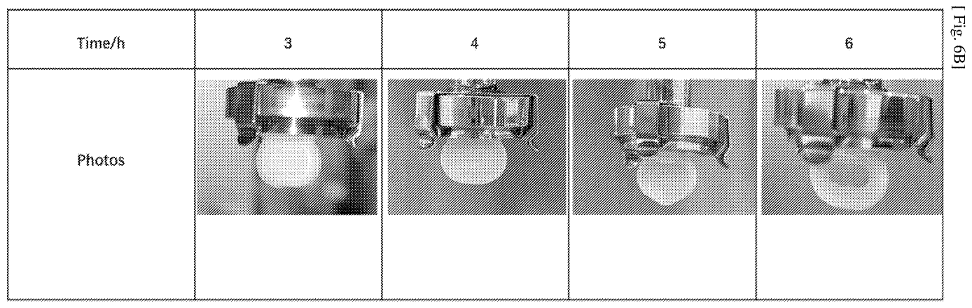

To mimic in vivo gastric versus intestinal milieu conditions, a two-stage dissolution was conducted in biorelevant media of FaSSGF and FaSSIF sequentially. For the first 0.5 hours, 250 mL FaSSGF was used to mimic gastric conditions, the dissolution medium was then switched to 250 mL FaSSIF, to mimic intestinal conditions. A USP apparatus II (paddle) operated at a speed of 100 RPM and maintained at a temperature of 37±0.5° C. inside the vessel was used. The cumulative dissolution profile of tofacitinib from each tested oral drug dosage form are provided in FIG. 5 (oral drug dosage form of Tables 1A and 1B), FIG. 6 (oral drug dosage form of Tables 2A and 2B), FIG. 7 (oral drug dosage form of Tables 3A and 3B), and FIG. 8 (oral drug dosage form of Tables 4A and 4B). The data in FIG. 5 represent quadruplicate testing and the results are the mean plus-or-minus the standard deviation between the replicates. As shown in FIG. 5, the delay of drug release from the oral drug dosage form was about 4.5 hours. The data in FIG. 6A represent triplicate testing and the results are the mean plus-or-minus the standard deviation between the replicates. As shown in FIG. 6A, the delay of drug release from the oral drug dosage form was about 4 hours. The phenomena in the dissolution process were observed and photos were taken which were shown in FIG. 6B, indicating that the surface of the drug dosage form of the present application did not gelatinize during the dissolution process The data in FIG. 7 represent triplicate testing and the results are the mean plus-or-minus the standard deviation between the replicates. As shown in FIG. 7, the delay of drug release from the oral drug dosage form was about 4.5 hours. The data in FIG. 8 represent triplicate testing and the results are the mean plus-or-minus the standard deviation between the replicates. As shown in FIG. 8, the delay of drug release from the oral drug dosage form was about 5 hours. Corresponding X-ray analysis results of the structural components within each oral drug dosage form are provided on FIGS. 5, 6, and 8. These in vitro results demonstrate that drug release from the oral drug dosage forms can be effectively delayed when assessed in biorelevant media with a reasonable volume of 250 mL in each of the simulated fluids, indicating the potential for colon-targeted delivery of this prototype tablet with the layers' thickness-delay time relationship for optimization.

PK and X-ray studies were performed using beagle dogs. The beagle dogs (non-naive, age: 15~16 months; sex: male; weight: 7.9~8.8 kg) were purchased from Beijing Marshall Biotechnology Co., Ltd. (Beijing, China). The dogs were hosted at the animal facility of 3D BioOptima Co., Ltd., according to FDA regulations for the maintenance and experimentation on animals. Each animal was housed individually in a stainless-steel cage in controlled environment (temperature 16~26° C., humidity 40%~70%, 12 h light/12 h dark cycles). Each dog was labeled with ear tattoo. Prior to dosing, the dogs were transferred to facility of Suzhou Chuxin Pet Hospital Co., Ltd. and acclimated for 2 days, where the animal experiment and husbandry were conducted.

Dogs were fasted and injected with pentagastrin before oral dosing to adjust stomach to low pH to mimick pH conditions in the fasted human stomach. For animals orally administered one oral drug dosage form, X-ray scans were scheduled to be recorded, and X-ray images collection were extended until barium sulfate in the oral drug dosage form disappeared, which demonstrated drug release completion. In parallel, blood samples were collected from beagle dog jugular vein. For animals orally administered reference drug Xeljanz® XR, only blood samples were collected from beagle dog jugular vein. The PK parameters were calculated using a non-compartmental model with WinNonlin version 8.0 (Pharsight, St. Louis, USA). The X-ray images were used to evaluate the location, transit time and release properties in the GI tract at different timepoints. PK results are provided in Table 9.

TABLE 9
PK parameters measured from beagle dogs administered an oral
drug dosage form comprising tofacitinib.

| | | | *Tables 1A | *Tables 3A | Tables 4A |
|---|---|---|---|---|---|
| | | Reference | and 1B | and 3B | and 4B |
| PK parameter | | (n = 6) | (n = 2) | (n = 2) | (n = 6) |
| ^{#}T_{det} | h | NA | 7 (7) | 6.75 (6.0-7.5) | 8 (6.0-24.0) |
| median (range) | | | | | |
| ^{#}Tₘₐₓ | h | 3 (2.0-4.0) | 9 (9) | 7 (6.0-8.0) | 9 (6.5-24.0) |
| median (range) | | | | | |
| Cₘₐₓ (Mean ± SD) | ng/ml | 74.4 ± 21.5 | 7.5 | 45.0 | 22.3 ± 14.8 |
| AUC₀₋ₜ (Mean ± SD) | ng*h/mL | 242 ± 55.9 | 33.8 | 150 | 98.7 ± 81.3 |
| AUC_{0-inf} (Mean ± SD) | ng*h/mL | 252 ± 56.3 | 36.6 | 155 | 121 ± 75.6 |
| Cₘₐₓ/dose (Mean ± SD) | kg*ng/ml/mg | 56.4 ± 16.0 | 72.1 | 107.9 | 33.9 ± 22.9 |
| AUC₀₋ₜ/dose (Mean ± SD) | h*kg*ng/mL/mg | 183 ± 42.5 | 327.5 | 360 | 150 ± 125 |
| AUC_{0-inf}/dose (Mean ± SD) | h*kg*ng/mL/mg | 191 ± 42.4 | 354.7 | 372 | 184 ± 116 |

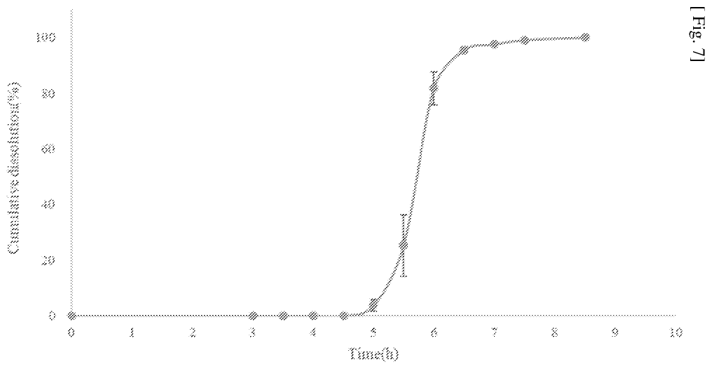

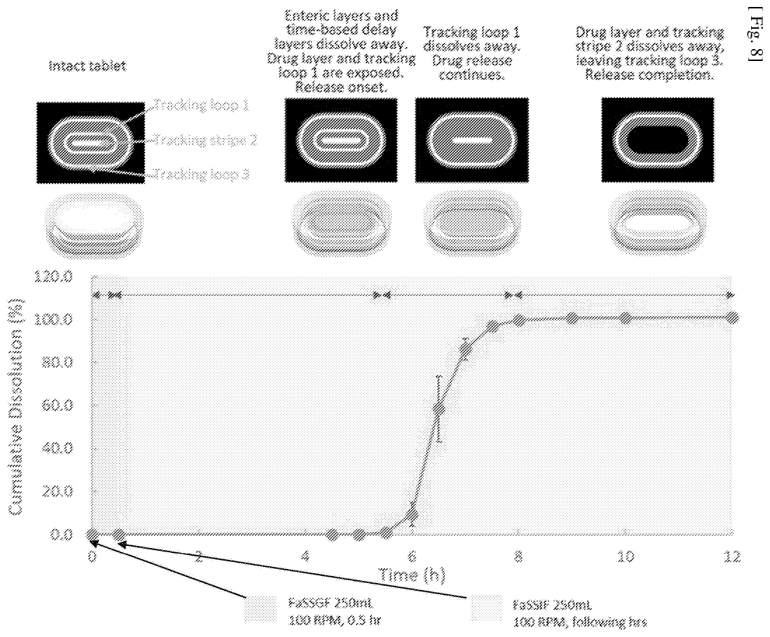

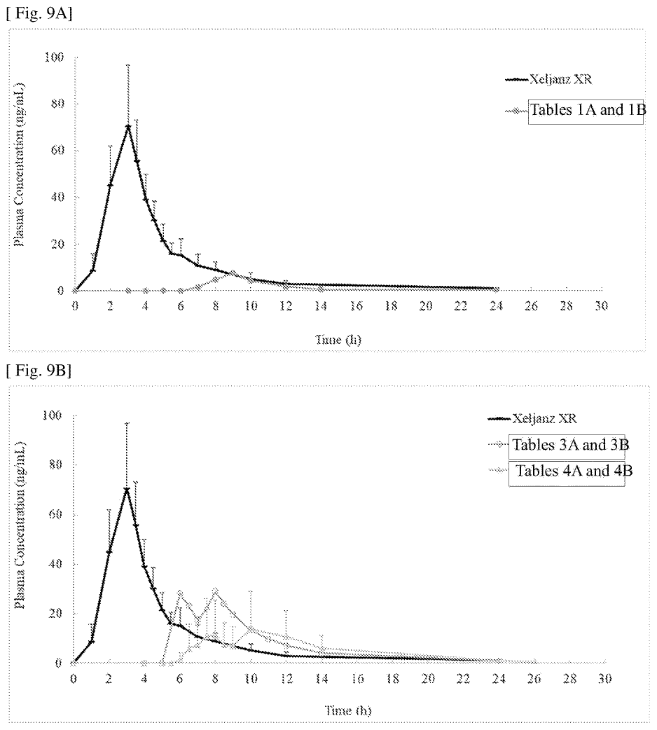

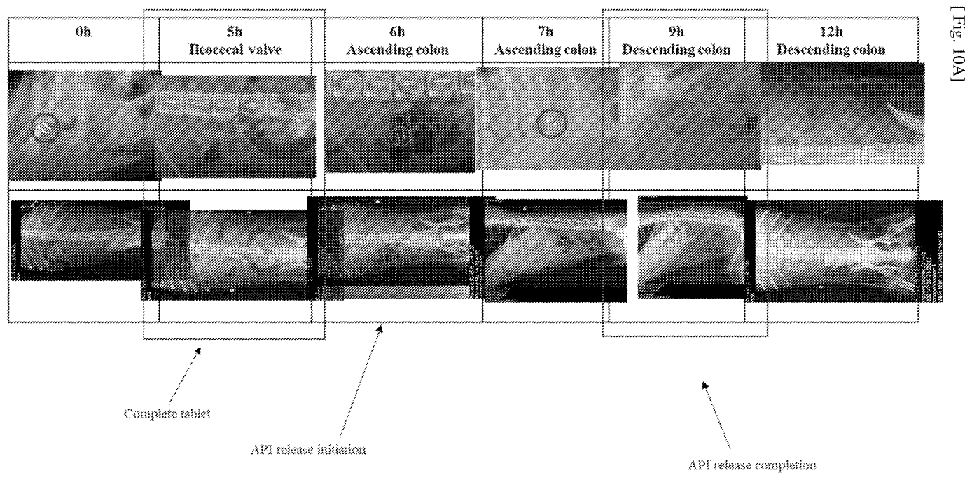

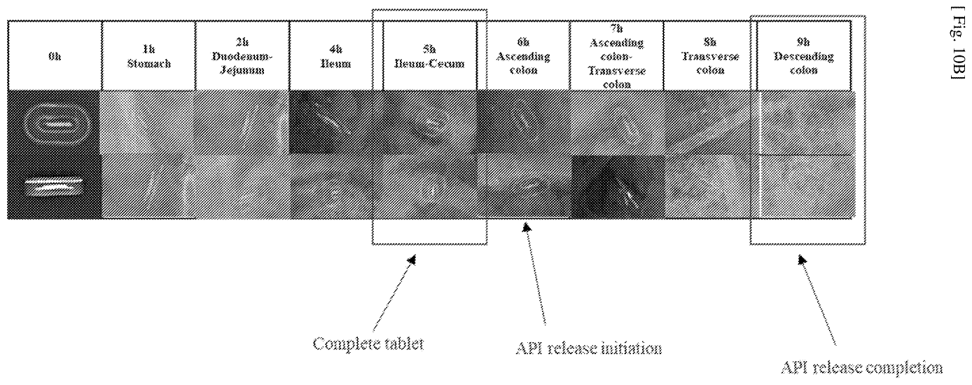

Note:
^{#}For T_{det}, and Tₘₐₓ, Median values were presented instead of Mean and Range instead of SD.
NA: not applicable.
*As the numbers of dogs for dosage forms of Tables 1A and 1B and Ta bles 3A and 3B are two, the PK parameters are the mean value rather than the SD value.
PK curves of the tested oral drug dosage forms are provided in FIGS. 9A and 9B. X-ray images are provided in FIGS. 10A (Tables 1A and 1B) , 10B (Tables 3A and 3B) , and 10C (Tables 4A and 4B) . Each X-ray image indicates time of capture, drug release initiation and drug release completion.

### Example 2

This example demonstrates the design, production, and testing of an oral drug dosage form described herein comprising tofacitinib citrate.

Release characteristics of four oral drug dosage form provided in Tables 5A-8B were first evaluated in vitro. The cumulative dissolution testing was performed in two stages—an acid stage to simulate the stomach and a buffer stage to simulate conditions downstream of the stomach. In the acid stage, 750 mL of 0.1 N hydrochloric acid was placed in the testing vessel, and the rest of the apparat us was assembled. The medium was equilibrated to 37±0.5° C. One test oral drug dosage form was placed in the basket, the vessel was covered, and the apparatus was operated at 100 rpm. After 2 hours, 4.5 mL of solution was removed and filtered with 0.22 μm hydrophilic syringe filter. To transition to the buffer stage, the apparatus was stopped to add 250 mL 0.2 mol/L sodium phosphate solution at 37° C. into acid medium immediately. The solution was then mixed and the pH was adjusted to 6.8±0.05 with 2 mol/L hydrochloric acid solution or 2 mol/L sodium hydroxide. The apparatus was restarted and run as described above. 4.5 mL of solution at different timepoints was removed and filtered with 0.2 2 μm hydrophilic syringe filter. The removed solutions were then tested to produce cumulative dissolution profiles.

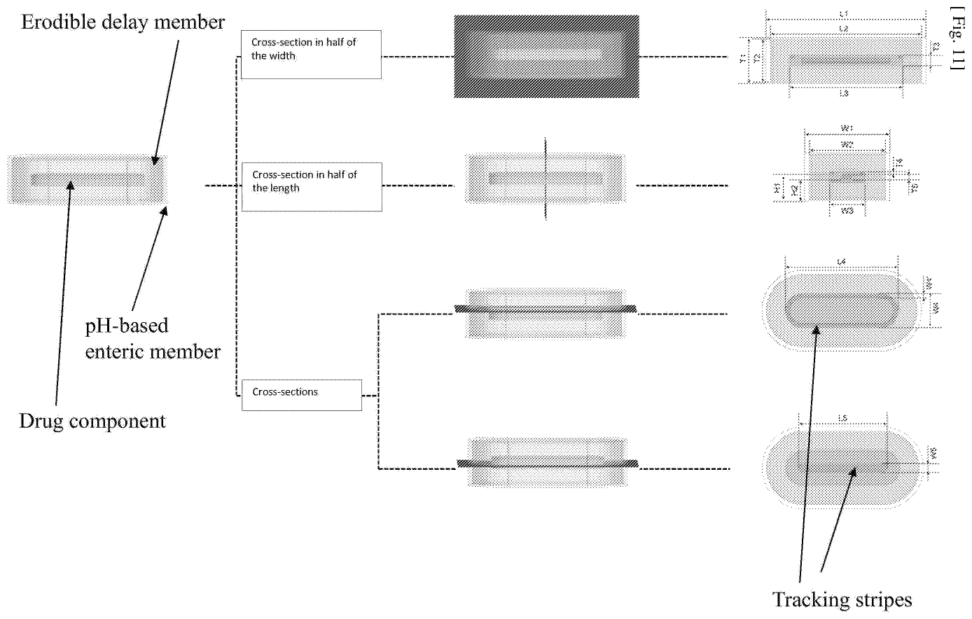

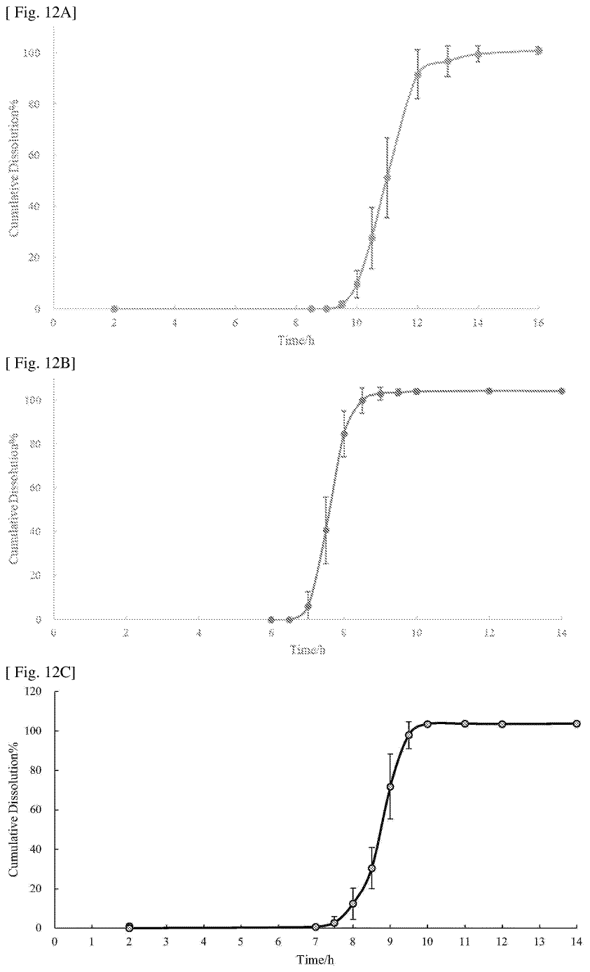

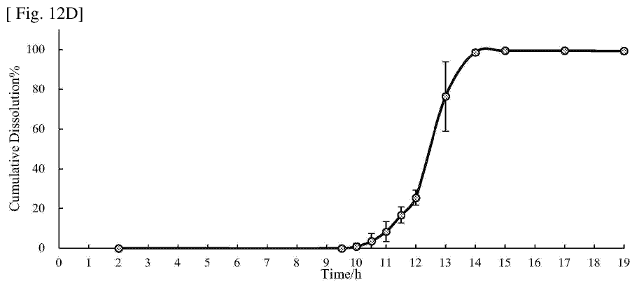

The cumulative dissolution profile of tofacitinib from each tested oral drug dosage form are provided in FIG. 12A (oral drug dosage form of Tables 5A and 5B), FIG. 12B (oral drug dosage form of Tables 6A and 6B), FIG. 12C (oral drug dosage form of Tables 7A and 7B), and FIG. 12D (oral drug dosage form of Tables 8A and 8B). The data in FIGS. 12A-12D represent 5 replicates of testing and the results are the mean plus-or-minus the standard deviation between the replicates. As shown in FIG. 12A, the delay of drug release from the oral drug dosage form was about 7.5 hours. As shown in FIG. 12B, the delay of drug release from the oral drug dosage form was about 5 hours. As shown in FIG. 12C, the delay of drug release from the oral drug dosage form was about 5.5 hours. As shown in FIG. 12D, the delay of drug release from the oral drug dosage form was about 8.5 hours.

These in vitro results demonstrate that drug release from the oral drug dosage forms can be effectively delayed when assessed in biorelevant media with a reasonable volume of 250 mL in each of the simulated fluids, indicating the potential for colon-targeted delivery of this prototype tablet with the layers' thickness-delay time relationship for optimization.

PK and X-ray studies were performed using beagle dogs. The beagle dogs (non-naive, age: 15~16 months; sex: male; weight: 7.9~8.8 kg) were purchased from Beijing Marshall Biotechnology Co., Ltd. (Beijing, China). The dogs were hosted at the animal facility of 3D BioOptima Co., Ltd., according to FDA regulations for the maintenance and experimentation on animals. Each animal was housed individually in a stainless-steel cage in controlled environment (temperature 16~26° C., humidity 40%~70%, 12 h light/12 h dark cycles). Each dog was labeled with ear tattoo. Prior to dosing, the dogs were transferred to facility of Suzhou Chuxin Pet Hospital Co., Ltd. and acclimated for 2 days, where the animal experiment and husbandry were conducted.

Dogs were fasted and injected with pentagastrin before oral dosing to adjust stomach to low pH to mimick pH conditions in the fasted human stomach. For animals orally administered one oral drug dosage form, X-ray scans were scheduled to be recorded, and X-ray images collection were extended until barium sulfate in the oral drug dosage form disappeared, which demonstrated drug release completion. In parallel, blood samples were collected from beagle dog jugular vein. For animals orally administered reference drug Xeljanz® XR, only blood samples were collected from beagle dog jugular vein. The PK parameters were calculated using a non-compartmental model with WinNonlin version 8.0 (Pharsight, St. Louis, USA). The X-ray images were used to evaluate the location, transit time and release properties in the GI tract at different timepoints. PK results are provided in Table 10.

TABLE 10
PK parameters measured from beagle dogs administered
an oral drug dosage form comprising tofacitinib.

| | | | *Tables 5A | *Tables 6A |
|---|---|---|---|---|
| | | Reference | and 5B | and 6B |
| PK parameter | | (n = 6) | (n = 5) | (n = 5) |
| ^{#}T_{det} | h | NA | 9 (8.0-11.0) | 7 (6.0-8.0) |
| median (range) | | | | |
| ^{#}Tₘₐₓ | h | 3 (2.0-4.0) | 12 (10.0-14.0) | 8 (7.0-9.0) |
| median (range) | | | | |
| Cₘₐₓ (Mean ± SD) | ng/ml | 74.4 ± 21.5 | 28 ± 22.6 | 40.1 ± 14.4 |
| AUC₀₋ₜ (Mean ± SD) | ng*h/mL | 242 ± 55.9 | 40.2 ± 31.7 | 171 ± 33.2 |
| AUC_{0-inf} (Mean ± SD) | ng*h/mL | 252 ± 56.3 | 141 ± 69.4 | 184 ± 50.0 |
| Cₘₐₓ/dose (Mean ± SD) | kg*ng/ml/mg | 56.4 ± 16.0 | 151 ± 66.4 | 56.9 ± 23.5 |
| AUC₀₋ₜ/dose (Mean ± SD) | h*kg*ng/ml/mg | 183 ± 42.5 | 205 ± 107 | 240 ± 52.1 |
| AUC_{0-inf}/dose (Mean ± SD) | h*kg*ng/ml/mg | 191 ± 42.4 | 218 ± 102 | 259 ± 81.3 |

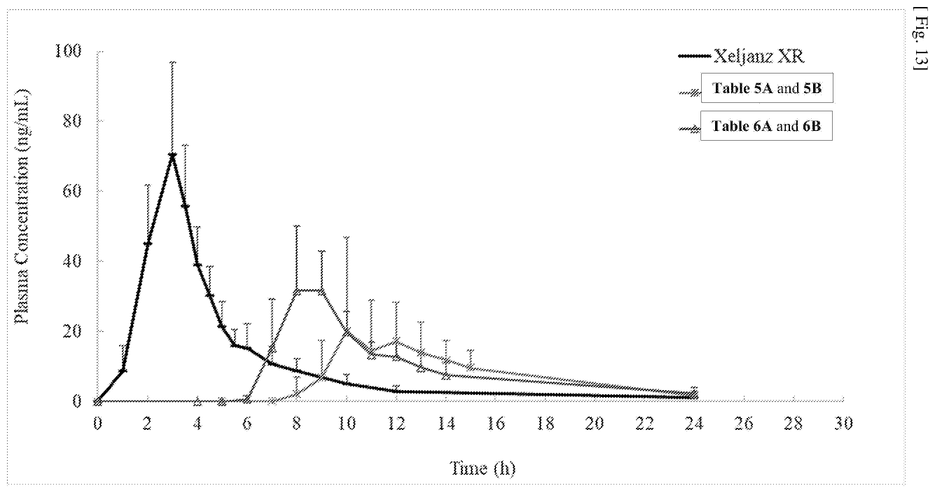

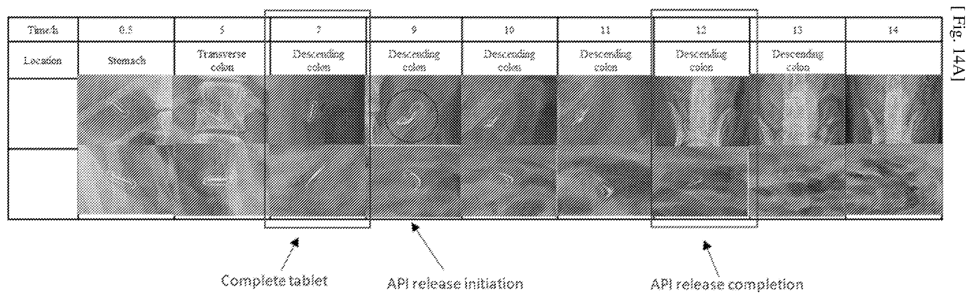

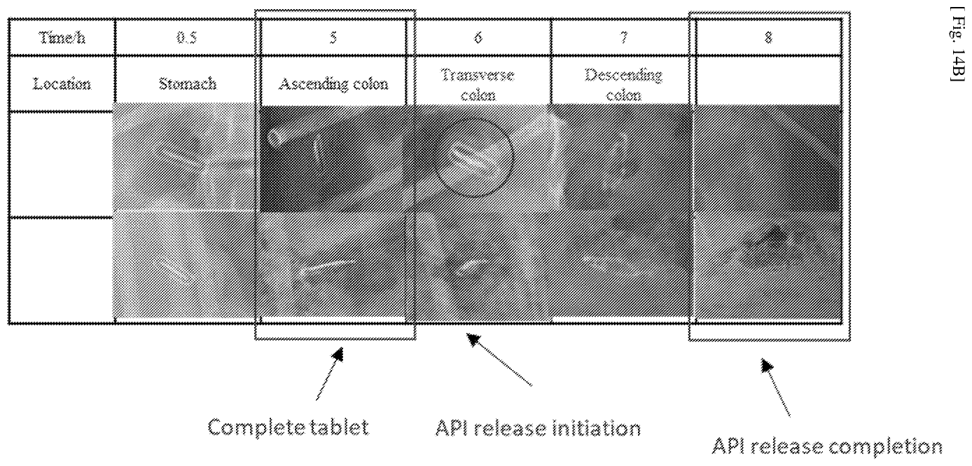

Note:
^{#}For T_{det}, and Tₘₐₓ, Median values were presented instead of Mean and Range instead of SD.
NA: not applicable.
PK curves of the tested oral drug dosage forms are provided in FIG. 13. X-ray images are provided in FIGS. 14A (Tables 5A and 5B, FIG. 11) , and 14B (Tables 6A and 6B, FIG. 11) . Each X-ray image indicates time of capture, drug release initiation and drug release completion.

The two candidate tablets were administered to healthy subjects to validate the colon-targeted drug delivery in vivo. The study was an open label, randomized, single dose, three period, three sequence, three-way crossover X-ray imaging and pharmacokinetic study in healthy subjects. Twelve healthy volunteers were enrolled into the study. Candidate tablet 1 and 2, and the reference of the model drug were administered in the morning under fasted conditions according to randomization plan. There were at least 4 days wash-out between each treatment. X-ray images and PK samples were collected at the predetermined timepoints. Tablet intestinal transit and drug release properties were evaluated based on X-ray images. The drug plasma concentration was determined by an LC-MS/MS analytical method. PK results in healthy volunteers are provided in Table 11.

TABLE 11
PK parameters measured from healthy volunteers administered
an oral drug dosage form comprising tofacitinib.

| | | Reference | Tablet 5A and | Tablet 6A and |
|---|---|---|---|---|
| PK parameters | | (n = 12) | 5B (*n = 9) | 6B (n = 12) |
| T_{lag} | h | 0.0 (0.0-0.0) | 8.0 (7.0-11.0) | 7.0 (5.0-8.0) |
| Tₘₐₓ | h | 3.0 (2.0-5.0) | 14.0 (11.0-16.0) | 11.0 (9.0-14.0) |
| Cₘₐₓ | ng/mL | 65.8 ± 22.7 | 17.2 ± 5.53 | 20.0 ± 5.80 |
| AUC₀₋ₜ | ng*h/mL | 525 ± 140 | 206 ± 69.2 | 231 ± 74.8 |
| AUC_{0-inf} | ng*h/mL | 530 ± 141 | 211 ± 70.7 | 234 ± 75.3 |

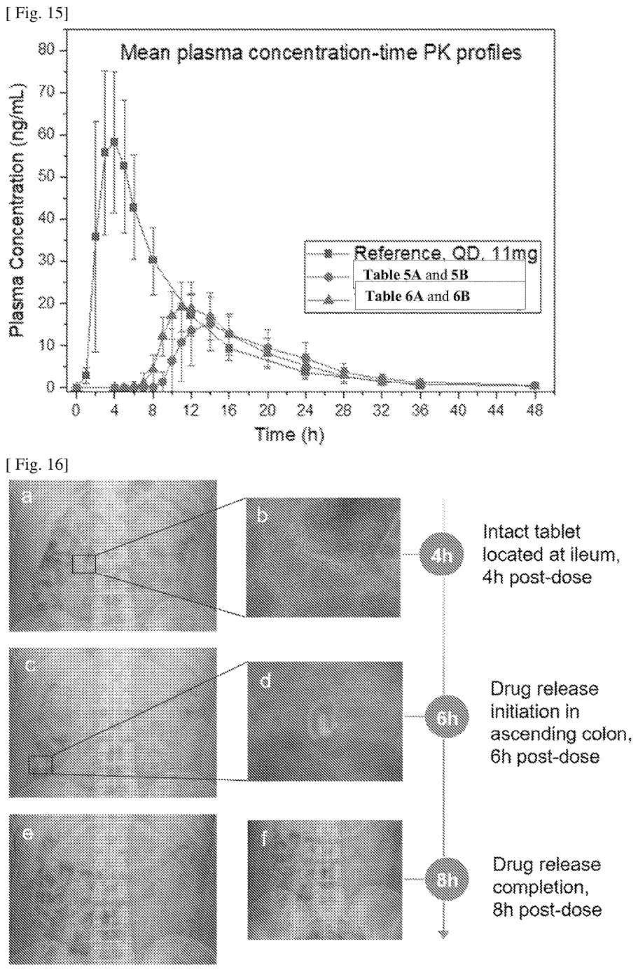

Note:
*n = 9 for Tablet 5A and 5B since the plasma concentration for three subjects were below the lower limit of quantification PK curves of the tested oral drug dosage forms are provided in FIG. 15. X-ray images are provided in FIG. 16 (Tables 6A and 6B, FIG. 11) . Each X-ray image indicates time of capture, drug release initiation and drug release completion.

The X-ray imaging results showed that the candidate tablet could be tracked in the gastrointestinal tract. Results from the majority subjects (8 out of n=12 for both test treatments) suggested that both candidate tablets released the drug within the colon region: Tablet 5A and 5B (3 subjects at ascending colon, 2 subjects at transverse colon, 1 subject at transverse to descending colon, 1 subject at descending colon, 1 subject at descending to sigmoid colon) while tablet 6A and 6B (7 subjects at ascending colon, 1 subject at transverse colon).

The pharmacokinetic results reflected the delayed release properties of the candidate tablets with a median (range) absorption T_{lag} of 8.0 h (7.0-11.0 h), 7.0 h (5.0-8.0 h) and 0.0 h (0.0-0.0 h) for tablets 5A and 5B, 6A and 6B and the reference tablet, respectively, which confirmed the colon release property of 3D printed test formulations.

The drug release and tracking agent images demonstrated that the release location/time occurred as predicted for the 3D printed tablets. The MED® 3D printed tablets with a sequential two-step control mechanism can precisely deliver drug to the colon in human, suggesting its potential application in IBD therapy.

1. An oral drug dosage form configured to release a drug at a desired location in the colon of an individual, the oral drug dosage form comprising:
a drug component comprising an erodible material admixed with the drug; and
a delay component not admixed with the drug,
wherein the delay component is configured to prevent release of the drug from the drug component until the oral drug dosage form reaches the colon of the individual following administration, and
wherein the delay component comprises:
a pH-based enteric member comprising an erodible material configured to erode at or above a pre-determined pH value.

2. The oral drug dosage form of claim 1, wherein the delay component further comprises an erodible delay member, and wherein the oral drug dosage form is configured such that the pH-based enteric member erodes prior to the erodible delay member.

3. The oral drug dosage form of claim 2, wherein the pH-based enteric member prevents erosion, at least in part, of the erodible delay member, and the erodible delay member prevents erosion, at least in part, of the drug component.

4. The oral drug dosage form of any one of claims 1-3, further comprising a second drug.

5. The oral drug dosage form of claim 4, wherein the second drug is admixed with the drug component.

6. The oral drug dosage form of claim 4, further comprising a second drug component comprising an erodible material admixed with the second drug, wherein the delay component is configured to prevent release of the second drug from the second drug component until the oral drug dosage form reaches the colon of the individual following administration.

7. The oral drug dosage form of claim 6, wherein the delay component comprises a second erodible delay member, wherein the oral drug dosage form is configured such that the pH-based enteric member erodes prior to the erodible delay member, the erodible delay member erodes prior to the second drug component, the second drug component erodes prior to the second erodible delay member, and the second erodible delay member erodes prior to the drug component.

8. The oral drug dosage form of claim 7, wherein the pH-based enteric member prevents erosion, at least in part, of the erodible delay member, the erodible delay member prevents erosion, at least in part, of the second drug component, the second drug component prevents erosion, at least in part, of the second erodible delay member, and the second erodible delay member prevents erosion, at least in part, of the drug component.

9. The oral drug dosage form of claim 7 or 8, wherein the erodible mate rial of the erodible delay member and the erodible material of the second erodible delay member are the same.

10. The oral drug dosage form of claim 7 or 8, wherein the erodible mate rial of the erodible delay member and the erodible material of the second erodible delay member are different.

11. The oral drug dosage form of any one of claims 2-10, further comprising a bacterial degradation moiety.

12. The oral drug dosage form of claim 11, wherein the bacterial degradation moiety is admixed with the erodible material of the erodible delay member.

13. The oral drug dosage form of claim 11, wherein the bacterial degradation moiety is admixed with the drug component.

14. The oral drug dosage form of any one of claims 11-13, wherein the delay component further comprises a bacterial degradation member comprising an erodible material admixed with the bacterial degradation moiety, wherein the oral drug dosage form is configured such that the pH-based enteric member erodes prior to the erodible delay member, the erodible delay member erodes prior to the bacterial degradation member, and the bacterial degradation member erodes prior to the drug component.

15. The oral drug dosage form of claim 14, wherein the pH-based enteric member prevents erosion, at least in part, of the erodible delay member, the erodible delay member prevents erosion, at least in part, of the bacterial degradation member, and the bacterial degradation member prevents erosion, at least in part, of the drug component.

16. The oral drug dosage form of any one of claims 11-15, wherein the bacterial degradation moiety is digestible by one or more microorganisms present in the colon of the individual.

17. The oral drug dosage form of any one of claims 11-16, wherein the bacterial degradation moiety comprises one or more saccharides.

18. The oral drug dosage form of claim 17, wherein the one or more saccharides comprises any one or more of a sucrose, glucose, xylose, fructose, maltose, galactose, pectin, galactomannan, dextran, inulin, chitosan, carrageenan, cellulose acetate propionate (CAP), peptidoglycan, gellan, xanthan gum, lentinan, psyllium polysaccharide, maize bran arabinoxylan, alginate, hyaluronic acid, fucoidan, shellac, agar, or maltodextrin.

19. The oral drug dosage form of any one of claims 14-18, wherein the bacterial degradation member is a layer, and wherein the layer has a thickness, as based on the direction of erosion, of about 0.1 mm to about 5 mm.

20. The oral drug dosage form of any one of claims 14-19, wherein the bacterial degradation member comprises a surface area exposed to a gastrointestinal fluid in the individual of about 10 mm² to about 400 mm².

21. The oral drug dosage form of any one of claims 1-20, wherein the delay component does not comprise an erodible delay member.

22. The oral drug dosage form of claim 21, wherein the erodible material of the pH-based enteric member comprises one or more of a hypromellose acetate succinate, hydroxypropyl methyl cellulose phthalate, cellulose acetate propionate (CAP), poly (methacrylic acid-co-ethyl acrylate), or polyvinyl acetate phthalate (PVAP).

23. The oral drug dosage form of claim 22, wherein the hypromellose acetate succinate is HPMCAS LG/LF, HPMCAS MG/MF, or HPMCAS HG/LF.

24. The oral drug dosage form of claim 22, wherein the hydroxypropyl methyl cellulose phthalate is HPMCP HP-50 or HPMCP HP-55.

25. The oral drug dosage form of claim 22, wherein the poly (methacrylic acid-co-ethyl acrylate) is Eudragit L100-55 or Eudragit L100.

26. The oral drug dosage form of any one of claims 1-25, wherein the drug component comprises a top surface, a bottom surface, and one or more side surfaces, and wherein the drug component is embedded in the delay component such that erosion of the delay component, or an aspect thereof, exposes at least two surfaces of any of the top surface, the bottom surface, or at least one of the one or more side surfaces to gastrointestinal fluid of the individual.

27. The oral drug dosage form of any one of claims 1-26, wherein the oral drug dosage form does not comprise a shell.

28. The oral drug dosage form of any one of claims 1-25, wherein the oral drug dosage form further comprises a shell not admixed with the drug, wherein the oral drug dosage form is configured such that the shell, or a portion thereof, and the delay member prevent erosion of the drug component.

29. The oral drug dosage form of claim 28, wherein the direction of erosion of the delay component and/or the drug component are based on the configuration of the shell.

30. The oral drug dosage form of any one of claims 1-29, further comprising a protease scavenger and/or a protease inhibitor.

31. The oral drug dosage form of claim 30, wherein the protease scavenger or the protease inhibitor is admixed with the delay component, or a portion thereof.

32. The oral drug dosage form of claim 30 or 31, wherein the protease scavenger or the protease inhibitor is admixed in the erodible material of the erodible delay member.

33. The oral drug dosage form of any one of claims 1-32, wherein the oral drug dosage form is configured to release the drug at, near, or downstream of any one of the cecum, ascending colon, hepatic flexure, transverse colon, splenic flexure, descending colon, or sigmoid colon.

34. The oral drug dosage form of any one of claims 1-33, wherein at least about 95% of the drug in the oral drug dosage form is released from the oral drug dosage form in the colon of the individual.

35. The oral drug dosage form of any one of claims 1-34, wherein less than about 2% of the drug in the oral drug dosage form is released from the oral drug dosage form outside of the colon of the individual.

36. The oral drug dosage form of any one of claims 1-35, wherein all of the drug in the oral drug dosage form is released from the oral drug dosage form in the colon of the individual.

37. The oral drug dosage form of any one of claims 2-36, wherein the drug component and/or the delay component are in the form of one or more layers.

38. The oral drug dosage form of claim 37, wherein the layer of the pH-based enteric member comprises a top surface and a bottom surface,
wherein the layer of the erodible delay member comprises a top surface and a bottom surface,
wherein the layer of the drug component comprises a top surface and a bottom surface,
wherein the bottom surface of the layer of the pH-based enteric member is in contact with the top surface of the layer of the erodible delay member, and
wherein the bottom surface of the layer of the erodible delay member is in contact with the top surface of the layer of the drug component.

39. The oral drug dosage form of any one of claims 1-38, wherein the oral drug dosage form is configured to have more than one compartment, and wherein at least one compartment comprises the drug component and the delay component.

40. The oral drug dosage form of any one of claims 1-39, wherein the pH-based enteric member erodes at or above a pH value of about 5.5.

41. The oral drug dosage form of any one of claims 1-40, wherein the p H-based enteric member is a layer having a thickness, as based on the direction of erosion, of about 0.1 mm to about 5 mm.

42. The oral drug dosage form of any one of claims 1-41, wherein the pH-based enteric member comprises a surface area exposed to a gastrointestinal fluid in the individual of about 10 mm² to about 400 mm².

43. The oral drug dosage form of any one of claims 1-42, wherein exterior surfaces of the oral drug dosage form, prior to administration to the individual, comprise the pH-based enteric member, and, optionally, the shell.

44. The oral drug dosage form of any one of claims 1-43, wherein the pH-based enteric member comprises a thermoformable material.

45. The oral drug dosage form of any one of claims 1-44, wherein the pH-based enteric member comprises one or more of: stearic acid, copolyvidone, polyvinylpyrrolidone-co-vinyl-acetate, polyvinylpyrrolidone-polyvinyl acetate copolymer, crospovidone, polyvinylpyrrolidone, hydroxypropyl cellulose, hydroxylpropyl methylcellulose or hypromellose, hydroxypropyl methylcellulose phthalate, methyl cellulose, methacrylic acid copolymer, poly (methacrylic acid-co-ethyl acrylate), poly (butyl methacrylate-co-(2-dimethylaminoethyl) methacrylate-co-methyl methacrylate), poly (dimethylaminoethylmethacrylate-co-methacrylic esters), poly (ethyl acrylate-co-methyl methacrylate-co-trimethylammonioethyl methacrylate chloride), poly (methyl acrylate-co-methyl methacrylate-co-methacrylic acid), poly (methacrylic acid-co-methylmethacrylate), poly (methacylic acid-co-ethyl acrylate), poly (methacylic acid-co-methyl methacrylate), polyethylene oxide, polyethylene glycol, polyvinyl caprolactam-polyvinyl acetate-polyethylene glycol graft copolymer, polyethylene glycol-polyvinyl alcohol graft copolymer, Kollicoat IR-polyvinyl alcohol, polyvinyl alcohol, aminoalkyl methacrylate copolymer E, hydroxypropyl methylcellulose acetate succinate or hypromellose acetate succinate, methacrylic ester copolymer, ammonioalkyl meth acrylate copolymer, ethyl cellulose, polyvinyl acetate and polyvinyl pyrrolidone, polyvinyl acetal diethyl aminolactate, polyvinyl acetal diethyl amino lactate, maltitol, lactose monohydrate, xylitol, isomalt, sucrose, glucose, dextran, or maltose.

46. The oral drug dosage form of any one of claims 2-45, wherein the erodible delay member prevents release of the drug from the drug component for at least about 10 minutes following contact of the erodible delay member to a gastrointestinal fluid in the individual.

47. The oral drug dosage form of any one of claims 2-46, wherein the erodible delay member is a layer having a thickness, as based on the direction of erosion, of about 0.1 mm to about 5 mm.

48. The oral drug dosage form of any one of claims 2-47, wherein the erodible delay member comprises a surface area exposed to a gastrointestinal fluid in the individual of about 10 mm² to about 400 mm².

49. The oral drug dosage form of any one of claims 2-48, wherein the erodible delay member comprises a thermoformable material.

50. The oral drug dosage form of any one of claims 2-49, wherein the erodible delay member comprises one or more of stearic acid, medium chain triglycerides, glyceryl distearate, propylene glycol monolaurate, propylene glycol caprylate, oleoyl polyoxyl-6 glycerides, PEG-6 STEARATE, PEG-32 STEARATE, linoleoyl polyoxyl-6 glycerides, lauroyl polyoxyl-32 glycerides, caprylocaproyl polyoxyl-8 glycerides, sorbitan monolaurate, sorbitan monopalmitate, sorbitan monostearate, sorbitan oleate, glyceryl monolinoleate, copolyvidone polyvinylpyrrolidone-co-vinyl-acetate, polyvinylpyrrolidone-polyvinyl acetate copolymer, crospovidone, polyvinylpyrrolidone, hydroxypropyl cellulose, hydroxylpropyl methylcellulose or Hypromellose hydroxypropyl methylcellulose phthalate, methyl cellulose, methacrylic acid copolymer, poly (methacrylic acid-co-ethyl acrylate), poly (butyl methacrylate-co-(2-dimethylaminoethyl) methacrylate-co-methyl methacrylate), poly (dimethylaminoethylmethacrylate-co-methacrylic esters), poly (ethyl acrylate-co-methyl methacrylate-co-trimethylammonioethyl methacrylate chloride), poly (methyl acrylate-co-methyl methacrylate-co-methacrylic acid), poly (methacrylic acid-co-methylmethacrylate), poly (methacylic acid-co-ethyl acrylate), poly (methacylic acid-co-methyl methacrylate), polyethylene oxide, polyethylene glycol, polyvinyl caprolactam-polyvinyl acetate-polyethylene glycol graft copolymer, polyethylene glycol-polyvinyl alcohol graft copolymer, Kollicoat IR-polyvinyl alcohol, polyvinyl alcohol, aminoalkyl methacrylate copolymer E, hydroxypropyl methylcellulose acetate succinate or hypromellose acetate succinate, methacrylic ester copolymer, ammonioalkyl methacrylate copolymer, ethyl cellulose, polyvinyl acetate and polyvinylpyrrolidone, polyvinyl acetal diethyl aminolactate, polyvinyl acetal diethyl amino lactate, maltitol, lactose monohydrate, xylitol, isomalt, sucrose, glucose, dextran, maltose, Lauroyl Polyoxyl-32 glycerides, caprylocaproyl polyoxyl-8 glycerides, polyoxyl (35) Castor oil, Vitamin E polyethylene glycol succinate, PEG-40 hydrogenated Castor oil, glyceryl monolinoleate, or glyceryl dibehenate.

51. The oral drug dosage form of any one of claims 1-50, wherein the drug component is not in direct contact with the pH-based enteric member.

52. The oral drug dosage form of any one of claims 1-51, wherein the drug component is a controlled-release drug component.

53. The oral drug dosage form of any one of claims 1-51, wherein the drug component is a sustained-release drug component.

54. The oral drug dosage form of any one of claims 1-51, wherein the drug component is an immediate-release drug component.

55. The oral drug dosage form of any one of claims 1-51, wherein the drug component is configured to release the drug from the oral drug dosage form for at least about 1 hour.

56. The oral drug dosage for of any one of claims 1-55, wherein the drug component is configured to provide a drug release profile comprising a zero-order release profile, first-order release profile, delayed release profile, pulsed release profile, iterative pulsed release profile, immediate release profile, or sustained release profile, or a combination thereof.

57. The oral drug dosage form of any one of claims 1-56, wherein the release of the drug is based, at least in part, on an in vitro dissolution study.

58. The oral drug dosage form of any one of claims 1-57, wherein the release of the drug is based, at least in part, on an in vivo dissolution study.

59. The oral drug dosage form of any one of claims 1-58, wherein the drug component has a drug mass fraction (m_{F}) of the drug of about 0.05 to about 0.6.

60. The oral drug dosage form of any one of claims 1-59, wherein the weight ratio of the drug component to the delay component is about 1:10 to about 10:1.

61. The oral drug dosage form of any one of claims 1-60, wherein the drug component is a layer having a thickness, as based on the direction of erosion, of about 0.1 mm to about 5 mm.

62. The oral drug dosage form of any one of claims 1-61, wherein the drug component comprises a surface area exposed to a gastrointestinal fluid in the individual of about 10 mm² to about 400 mm².

63. The oral drug dosage form of any one of claims 1-62, wherein the drug component comprises a thermoformable material.

64. The oral drug dosage form of any one of claims 1-63, wherein the drug component comprises one or more of copolyvidone, polyvinylpyrrolidone-co-vinyl-acetate, polyvinylpyrrolidone-polyvinyl acetate copolymer, polyvinylpyrrolidone, hydroxypropyl cellulose, hydroxylpropyl methylcellulose or Hypromellose, polyethylene oxide, polyethylene glycol, polyvinyl caprolactam-polyvinyl acetate-polyethylene glycol graft copolymer, polyethylene glycol-polyvinyl alcohol graft copolymer, Kollicoat IR-polyvinyl alcohol, polyvinyl alcohol, aminoalkyl methacrylate copolymer E, hydroxypropyl methylcellulose acetate succinate hypromellose acetate succinate, hydroxypropyl methylcellulose phthalate, maltitol, lactose monohydrate, xylitol, isomalt, sucrose, glucose, or dextran.

65. The oral drug dosage form of any one of claims 1-64, further comprising one to four additional drug components.

66. The oral drug dosage form of any one of claims 1-65, wherein the drug is an anti-inflammatory agent, non-steroidal anti-inflammatory agent, steroid, immunosuppressant, antibiotic, anti-neoplastic agent, analgesic, anaesthetic, anticonvulsant, antidiabetic agent, antihistamine, anti-infective, antineoplastic, antiparkinsonian agent, antirheumatic agent, appetite stimulant, appetite suppressant, blood modifier, bone metabolism modifier, cardiovascular agent, central nervous system depressant, central nervous system stimulant, decongestant, dopamine receptor agonist, electrolyte, gastrointestinal agent, immunomodulator, muscle relaxant, narcotic, parasympathomimetic, sympathomimetic, sedative, hypnotic, or vaccine.

67. The oral drug dosage form of claim 66, wherein the anti-inflammatory agent is a JAK inhibitor.

68. The oral drug dosage form of claim 67, wherein the JAK inhibitor is tofacitinib or a pharmaceutically acceptable salt thereof.

69. The oral drug dosage form of claim 66, wherein the anti-inflammatory agent is mesalazine, sulphasalazine, or balsalazide.

70. The oral drug dosage form of claim 66, wherein the non-steroidal anti-inflammatory agent is ibuprofen or diclofenac.

71. The oral drug dosage form of claim 66, wherein the steroid is prednisolone, budesonide, or fluticasone.

72. The oral drug dosage form of claim 57, wherein the immunosuppressant is azathioprine, cyclosporin, or methotrexate.

73. The oral drug dosage form of claim 66, wherein the antineoplastic agent is fluorouracil, methotrexate, dactinomycin, bleomycin, etoposide, taxol, vincristine, doxorubicin, cisplatin, daunorubicin, Etoposide, raltitrexed, or oxaliplatin, or a combination thereof.

74. The oral drug dosage form of any one of claims 1-73, wherein the oral drug dosage form comprises about 0.01 mg to about 500 mg of the drug.

75. The oral drug dosage form of any one of claims 1-74, further comprising an excipient.

76. The oral drug dosage form of claim 75, wherein the excipient comprises one or more of an absorption enhancer, pH-lowering agent, or a disintegrant.

77. The oral drug dosage form of claim 75 or 76, wherein the excipient is admixed in the drug component.

78. The oral drug dosage form of any one of claims 28-77, wherein the shell comprises an insulating material that is impermeable to bodily fluids.

79. The oral drug dosage form of claim 78, wherein the insulating material comprises one or more of an ethylcellulose (EC), dibutyl sebacate (DBS), and titanium dioxide.

80. The oral drug dosage form of claim 78 or 79, wherein the insulating material is a non-erodible material.

81. The oral drug dosage form of any one of claims 78-80, wherein the insulating material is an erodible material having a pH-based erosion and/or an erosion rate that allows for the complete re lease of the drug from the oral drug dosage form prior to exposure of the drug component to bodily fluids due to erosion of the shell.

82. The oral drug dosage form of any one of claims 1-81, further comprising a second pH-based enteric member.

83. The oral drug dosage form of claim 82, wherein the pH-based enteric member erodes from the oral drug dosage form at or above a pH value of about 5.5, or a portion thereof.

84. The oral drug dosage form of claim 82 or 83, wherein the second pH-based enteric member erodes from the oral drug dosage form at or above a pH value of about 6.8, or a portion thereof.

85. A commercial batch of an oral drug dosage form of any one of claims 1-84, wherein the commercial batch has a standard deviation of about 0.05 or less for each of the following:
an amount of a drug in the oral drug dosage form;
a weight of the oral drug dosage form;
a largest crossing dimension of the oral drug dosage form; and
a crossing dimension perpendicular to the largest crossing dimension of the oral drug dosage form.

86. The commercial batch of claim 85, wherein the commercial batch comprises at least about 1000 of the oral drug dosage forms.

87. A method of three-dimensional (3D) printing of an oral drug dosage form of any one of claims 1-84, the method comprising dispensing materials according to a layer-by-layer model of the oral drug dosage form to print the oral drug dosage form, wherein each layer of the layer-by-layer model is printed by dispensing, as necessary, for a layer:
(a) a shell material to form any portion of a shell in the layer;
(b) a drug component material to form any portion of a drug component in the layer;
(c) a pH-based enteric member material to form any portion of a pH-based enteric member in the layer;
(d) an erodible delay member material to form any portion of an erodible delay member in the layer;
(e) a bacterial degradation member material to form any portion of abacterial degradation member in the layer; and
(f) an excipient component material to form any portion of an excipient component in the layer.

88. The method of claim 87, further comprising generating the layer-by-layer model of the oral drug dosage form.

89. The method of claim 87 or 88, wherein the dispensing is via melt extrusion deposition (MED).

90. The method of any one of claims 87-89, wherein dispensing of the shell material, dispensing of the drug component material, dispensing of the pH-based enteric member material, dispensing of the erodible delay member material, and dispensing of the shell material are each performed by a different printing head.

91. A method of treating a condition in an individual, the method comprising administering to the individual an oral drug dosage form of any one of claims 1-84.

92. The method of claim 91, wherein the condition is selected from the group consisting of a gastrointestinal disorder, central nervous system (CNS) disorder, cardiovascular disease, hypertension, atherosclerosis, angina, arterial obstruction, peripheral arterial disease, myocardial pathology, arrhythmia, acute myocardial infarction, angina, cardiomyopathy, congestive heart failure, coronary artery disease (CAD), carotid artery disease, endocarditis, hypercholesterolemia, hyperlipidemia, peripheral artery disease (PAD), genitourinary disorders; erectile dysfunction, urinary organ diseases benign prostatic hypertrophy (BPH), renal tubular acidosis, diabetic nephropathy, glomerulonephritis, glomerulosclerosis, urinary tract infection, faecal incontinence, ocular disease glaucoma, blepharitis, ocular hypertension, retinopathy, conjunctivitis, scleritis, retinitis, keratitis, corneal ulcer, iritis, chorioretinal inflammation, macular edema, xerophthalmia, pulmonary disease asthma, pulmonary hypertension, acute respiratory distress syndrome, COPD, emphysema, pneumonia, tuberculosis, bronchitis, acute bronchitis, bronchiectasis, bronchiolitis, bronchopulmonary dysplasia, byssinosis, coccidioidomycosis (Cocci), cystic fibrosis, influenza, lung cancer, mesothelioma, metabolic disease, hypercalciuria, hyperglycemia, hyperinsulinemic hypoglycemia, hyperinsulinism, hyperlysinuria, and hypoglycemia.

93. The method of claim 92, wherein the gastrointestinal disorder is selected from the group consisting of irritable bowel disease (IBD), irritable bowel syndrome (IBS), constipation, diarrhea, infection, and carcinoma.

94. The method of claim 93, wherein the IBD is associated with Crohn's disease or ulcerative colitis.

95. The method of claim 93, wherein the carcinoma is colon cancer or colorectal cancer.

96. The method of claim 93, wherein the CNS disorder is selected from the group consisting of neurogenic pain, stroke, dementia, Alzheimer's disease, Parkinson's disease, neuronal degeneration, meningitis, spinal cord injury, cerebral vasospasm, and amyotrophic lateral sclerosis.

97. A method of providing local delivery of a drug to the colon of an in dividual, the method comprising administering to the individual an oral drug dosage form of any one of claims 1-84, wherein the individual is suffering from a gastrointestinal disorder.

98. A method of providing systemic delivery of a drug to an individual, the method comprising administering to the individual an oral drug dosage form of any one of claims 1-84, wherein the individual is suffering from a central nervous system (CNS) disorder, cardiovascular disease, hypertension, atherosclerosis, angina, arterial obstruction, peripheral arterial disease, myocardial pathology, arrhythmia, acute myocardial infarction, angina, cardiomyopathy, congestive heart failure, coronary artery disease (CAD), carotid artery disease, endocarditis, hypercholesterolemia, hyperlipidemia, peripheral artery disease (PAD), genitourinary disorders; erectile dysfunction, urinary organ diseases benign prostatic hypertrophy (BPH), renal tubular acidosis, diabetic nephropathy, glomerulonephritis, glomerulosclerosis, urinary tract infection, faecal incontinence, ocular disease glaucoma, blepharitis, ocular hypertension, retinopathy, conjunctivitis, scleritis, retinitis, keratitis, corneal ulcer, iritis, chorioretinal inflammation, macular edema, xerophthalmia, Pulmonary disease asthma, pulmonary hypertension, acute respiratory distress syndrome, COPD, emphysema, pneumonia, tuberculosis, bronchitis, acute bronchitis, bronchiectasis, bronchiolitis, bronchopulmonary dysplasia, byssinosis, coccidioidomycosis (Cocci), cystic fibrosis, influenza, lung cancer, mesothelioma, metabolic disease, hypercalciuria, hyper glycemia, hyperinsulinemic hypoglycemia, hyperinsulinism, hyperlysinuria, or hypoglycemia.

99. A method of designing an oral drug dosage form configured to release a drug at a desired gastrointestinal location in an individual,
wherein the oral drug dosage form comprises:
a drug component comprising:
a first erodible material admixed with a drug, and
a first erodible tracking stripe; and
a shell comprising a second tracking stripe;
a delay component not admixed with the drug, the delay component comprising:
a pH-based enteric member configured to erode at or above a pre-determined pH value,
wherein the pH based enteric member comprises a third erodible tracking stripe; and
an erodible delay member comprising a second erodible material, wherein the erodible delay member comprises a fourth erodible tracking stripe,
wherein the pH-based enteric member, alone or in combination with the shell, prevents erosion of the erodible delay member, and
wherein the erodible delay member, alone or in combination with the shell, prevents erosion of the drug component,
the method comprising:
(a) administering the oral drug dosage form to an individual;
(b) imaging the individual over a time course to obtain a location of the oral drug dosage form and a status of the first erodible tracking stripe, the second erodible tracking stripe, the third erodible tracking stripe, and the fourth tracking stripe; and
(c) adjusting the drug component and/or delay component and/or shell, or portion thereof, based on the location and the status of the first erodible tracking stripe, the second erodible tracking stripe, the third erodible tracking stripe, and the fourth track stripe to design the oral drug dosage form configured to release a drug at a desired gastrointestinal location in an individual.

100. The method of claim 99, wherein the adjusting the delay component comprises adjusting the pH-based enteric member by changing one or more of the composition, surface area exposed to bodily fluid following administration, or thickness of the pH-based enteric member.

101. The method of claim 99 or 100, wherein the adjusting the delay component comprises adjusting the erodible delay member by changing one or more of the composition, surface area exposed to bodily fluid foll owing administration, or thickness of the erodible delay member.

102. A method for treating ulcerative colitis, the method comprising administering to a human individual an oral drug dosage form of any one of claims 1-84, wherein the oral drug dosage form comprises a JAK inhibitor.

103. The method of claim 102, wherein the JAK inhibitor comprises tofacitinib.

104. The method of claim 103, wherein the oral drug dosage form comprises about 11 mg or less of tofacitinib.

105. The method of claim 103 or 104, wherein the oral drug dosage form comprises about 5.5 mg or less of tofacitinib.

106. The method of any one of claims 99-105, wherein the oral drug dosage form is administered once daily.

107. A method of designing an oral drug dosage form configured to release a drug at a desired gastrointestinal location in an individual,
wherein the oral drug dosage form comprises:
a drug component comprising:
a first erodible material admixed with a drug, and
a first erodible tracking stripe; and
a shell;
a delay component not admixed with the drug, the delay component comprising:
a pH-based enteric member configured to erode at or above a pre-determined pH value; and
an erodible delay member comprising a second erodible material,
wherein the pH-based enteric member, alone or in combination with the shell, prevents erosion of the erodible delay member, and
wherein the erodible delay member, alone or in combination with the shell, prevents erosion of the drug component,
the method comprising:
(a) administering the oral drug dosage form to an individual;
(b) imaging the individual over a time course to obtain a location of the oral drug dosage form and a status of the first erodible tracking stripe; and
(c) adjusting the drug component and/or delay component and/or shell, or portion thereof, based on the location and the status of the first erodible tracking stripe, to design the oral drug dosage form configured to release a drug at a desired gastrointestinal location in an individual.

108. The method of claim 107, wherein the first erodible material admixed with the drug and the first erodible tracking stripe are not in direct contact with one another.

109. The method of claim 107 or 108, wherein the shell comprises a second tracking stripe, and wherein the second tracking stripe is configured to be imaged by the imaging such as to obtain a status of the second tracking stripe.

110. The method of claim 109, wherein the adjusting is based on the location and the status of the first erodible tracking stripe and/or the second tracking stripe.

111. The method of any one of claims 107-110, wherein the pH-based enteric member comprises a third tracking stripe, and wherein the third tracking stipe is configured to be imaged by the imaging such as to obtain a status of the third erodible tracking stripe.

112. The method of claim 111, wherein the adjusting is based on the location and the status of the first erodible tracking stripe and/or the second tracking stripe and/or the third erodible tracking stripe.

113. The method of any one of claims 107-112, wherein the erodible delay member comprises a fourth tracking stripe, and wherein the fourth tracking stripe is configured to be imaged by the imaging such as to obtain a status of the fourth erodible tracking stripe.

114. The method of claim 113, wherein the adjusting is based on the location and the status of the first erodible tracking stripe and/or the second tracking stripe and/or the third erodible tracking stripe and/or the fourth erodible tracking stripe.

115. The method of any one of claims 107-114, wherein the imaging provides a location of the oral drug dosage form when the first erodible tracking stripe begins to erode.

116. The method of any one of claims 107-115, wherein the imaging provides a location of the oral drug dosage form when the first erodible tracking stripe has completely eroded.

117. The method of any one of claims 107-116, wherein the adjusting comprises adjusting the delay component, or portion thereof, to increase the delay of release of the drug from the oral drug dosage form based on the location and the status of the first erodible tracking stripe showing earlier than desired release.

118. The method of any one of claims 107-113, wherein the adjusting comprises adjusting the delay component, or portion thereof, to decrease the delay of release of the drug from the oral drug dosage form based on the location and the status of the first erodible tracking stripe showing later than desired release.

119. A method of designing an oral drug dosage form configured to release a drug at a desired gastrointestinal location in an individual,
wherein the oral drug dosage form comprises:
a drug component comprising a first erodible material admixed with a drug;
a shell; and
a delay component not admixed with the drug, the delay component comprising:
a pH-based enteric member configured to erode at or above a pre-determined pH value; and
an erodible delay member comprising a second erodible material,
wherein the pH-based enteric member prevents erosion of the erodible delay member, and
wherein the erodible delay member prevents erosion of the drug component,
wherein the oral drug dosage form comprises at least one tracking stripe positioned in a component thereof,
the method comprising:
(a) administering the oral drug dosage form to an individual;
(b) imaging the individual over a time course to obtain a location of the oral drug dosage form and a status of the at least one tracking stripe; and
(c) adjusting the drug component and/or delay component and/or shell, or portion thereof, based on the location and the status of the at least one tracking stripe, to design the oral drug dosage form configured to release a drug at a desired gastrointestinal location in an individual.

120. The method of claim 119, wherein a tracking stripe is located in, or near, the drug component.

121. The method of claim 119 or 120, wherein a tracking stripe is located in the shell.

122. The method of any one of claims 119-121, wherein a tracking stripe is located in the pH-based enteric member.

123. The method of any one of claims 119-122, wherein a tracking stripe is located in the erodible delay member.

124. The method of any one of claims 119-123, wherein at least one of the at least one tracking stripes is erodible.

125. A method of designing an oral drug dosage form configured to release a drug at a desired gastrointestinal location in an individual,
wherein the oral drug dosage form comprises:
a drug component comprising a first erodible material admixed with a drug; and
a delay component not admixed with the drug, the delay component comprising:
a pH-based enteric member configured to erode at or above a pre-determined pH value; and
an erodible delay member comprising a second erodible material,
wherein the pH-based enteric member prevents erosion of the erodible delay member, and
wherein the erodible delay member prevents erosion of the drug component,
wherein the oral drug dosage form comprises at least one tracking stripe positioned in a component thereof,
the method comprising:
(a) administering the oral drug dosage form to an individual;
(b) imaging the individual over a time course to obtain a location of the oral drug dosage form and a status of the at least one tracking stripe; and
(c) adjusting the drug component and/or delay component, or portion thereof, based on the location and the status of the at least one tracking stripe, to design the oral drug dosage form configured to release a drug at a desired gastrointestinal location in an individual.

126. The method of claim 125, wherein a tracking stripe is located in, or near, the drug component.

127. The method of claim 125 or 126, wherein a tracking stripe is located in the pH-based enteric member.

128. The method of any one of claims 125-127, wherein a tracking stripe is located in the erodible delay member.

129. The method of any one of claims 125-128, wherein at least one of the at least one tracking stripes is erodible.
